SEQUENCE LISTING (82 sequences)

SEQ ID NO: 1 - DNA, 25 bp, synthetic construct
  misc_feature 1..25: note = Primer
  misc_feature 1..22: note = DNA
  misc_feature 23..25: note = RNA
  source 1..25: mol_type = other DNA
acactctttccctacacgacgcggg

SEQ ID NO: 2 - DNA, 86 bp, synthetic construct
  misc_feature 1..86: note = Primer
  misc_feature 1: note = 5'-biotin
  misc_difference 40..49: note = N=any base
  misc_difference 50..54: note = V= any base other than T
  misc_difference 85: note = V= any base other than T
  misc_difference 86: note = N= any base
  source 1..86: mol_type = other DNA
acactctttccctacacgacgctcttccgatctatcacgnnnnnnnnnnvvvvvttttttttttttttttttttttttttttttvn

SEQ ID NO: 3 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctgtgaaannnnnnnnnnvvvvvttttttttttttttttttttttttttttttvn

SEQ ID NO: 4 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctgtagagnnnnnnnnnnvvvvvttttttttttttttttttttttttttttttvn

SEQ ID NO: 5 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctcaccggnnnnnnnnnnvvvvvttttttttttttttttttttttttttttttvn

SEQ ID NO: 6 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctcgatgtnnnnnnnnnnvvvvvttttttttttttttttttttttttttttttvn

SEQ ID NO: 7 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctagtcaannnnnnnnnnvvvvvttttttttttttttttttttttttttttttvn

SEQ ID NO: 8 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctatgagcnnnnnnnnnnvvvvvttttttttttttttttttttttttttttttvn

SEQ ID NO: 9 - DNA, 86 bp, synthetic construct
  misc_feature 1..86: note = Primer
  misc_feature 1: note = 5'-Biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctcggaatnnnnnnnnnnvvvvvttttttttttttttttttttttttttttttvn

SEQ ID NO: 10 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctttaggcnnnnnnnnnnvvvvvttttttttttttttttttttttttttttttvn

SEQ ID NO: 11 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctagttccnnnnnnnnnnvvvvvttttttttttttttttttttttttttttttvn

SEQ ID NO: 12 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctccaacannnnnnnnnnvvvvvttttttttttttttttttttttttttttttvn

SEQ ID NO: 13 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatcttacagcnnnnnnnnnnvvvvvttttttttttttttttttttttttttttttvn

SEQ ID NO: 14 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatcttgaccannnnnnnnnnvvvvvttttttttttttttttttttttttttttttvn

SEQ ID NO: 15 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctccgtccnnnnnnnnnnvvvvvttttttttttttttttttttttttttttttvn

SEQ ID NO: 16 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatcttaatcgnnnnnnnnnnvvvvvttttttttttttttttttttttttttttttvn

SEQ ID NO: 17 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatcttataatnnnnnnnnnnvvvvvttttttttttttttttttttttttttttttvn

SEQ ID NO: 18 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctacagtgnnnnnnnnnnvvvvvttttttttttttttttttttttttttttttvn

SEQ ID NO: 19 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctgacgacnnnnnnnnnnvvvvvttttttttttttttttttttttttttttttvn

SEQ ID NO: 20 - DNA, 86 bp, synthetic construct
  misc_feature 1..86: note = Primer
  misc_feature 1: note = 5'=biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctcactcannnnnnnnnnvvvvvttttttttttttttttttttttttttttttvn

SEQ ID NO: 21 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctctagctnnnnnnnnnnvvvvvttttttttttttttttttttttttttttttvn

SEQ ID NO: 22 - DNA, 86 bp, synthetic construct
  misc_feature 1..86: note = Primer
  misc_feature 1: note = 5'-biotin
  misc_difference 40..49: note = N=any base
  misc_difference 50..54: note = V= any base other than T
  misc_difference 85: note = V= any base other than T
  misc_difference 86: note = N=any base
  source 1..86: mol_type = other DNA
acactctttccctacacgacgctcttccgatctgccaatnnnnnnnnnnvvvvvttttttttttttttttttttttttttttttvn

SEQ ID NO: 23 - DNA, 86 bp, synthetic construct
  misc_feature 1..86: note = Primer
  misc_feature 1: note = 5'-primer
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatcttcattcnnnnnnnnnnvvvvvttttttttttttttttttttttttttttttvn

SEQ ID NO: 24 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctcattttnnnnnnnnnnvvvvvttttttttttttttttttttttttttttttvn

SEQ ID NO: 25 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctctatacnnnnnnnnnnvvvvvttttttttttttttttttttttttttttttvn

SEQ ID NO: 26 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctcagatcnnnnnnnnnnvvvvvttttttttttttttttttttttttttttttvn

SEQ ID NO: 27 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctgagtggnnnnnnnnnnvvvvvttttttttttttttttttttttttttttttvn

SEQ ID NO: 28 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatcttcgaagnnnnnnnnnnvvvvvttttttttttttttttttttttttttttttvn

SEQ ID NO: 29 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatcttcccgannnnnnnnnnvvvvvttttttttttttttttttttttttttttttvn

SEQ ID NO: 30 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctacttgannnnnnnnnnvvvvvttttttttttttttttttttttttttttttvn

SEQ ID NO: 31 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctattcctnnnnnnnnnnvvvvvttttttttttttttttttttttttttttttvn

SEQ ID NO: 32 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatctgtccgcnnnnnnnnnnvvvvvttttttttttttttttttttttttttttttvn

SEQ ID NO: 33 - DNA, 86 bp, synthetic construct
  misc_feature 1..86: note = Primer
  misc_feature 1: note = 5'-biotin
  misc_difference 40..49: note = N=any base
  misc_difference 50..54: note = V=any base other than T
  misc_difference 85: note = V=any base other than T
  misc_difference 86: note = N=any base
  source 1..86: mol_type = other DNA
acactctttccctacacgacgctcttccgatcttcggcannnnnnnnnnvvvvvttttttttttttttttttttttttttttttvn

SEQ ID NO: 34 - DNA, 22 bp, synthetic construct
  misc_feature 1..22: note = Primer
  misc_feature 1: note = 5'-biotin
  source 1..22: mol_type = other DNA
acactctttccctacacgacgc

SEQ ID NO: 35 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacactcgtggagcgacactctttccctacacgacgctcttccgatct

SEQ ID NO: 36 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacctacaagataacactctttccctacacgacgctcttccgatct

SEQ ID NO: 37 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacactatagtagctacactctttccctacacgacgctcttccgatct

SEQ ID NO: 38 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacactgcctggtggacactctttccctacacgacgctcttccgatct

SEQ ID NO: 39 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacacattatcctacactctttccctacacgacgctcttccgatct

SEQ ID NO: 40 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacgtccacttgtacactctttccctacacgacgctcttccgatct

SEQ ID NO: 41 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacactggaacagtaacactctttccctacacgacgctcttccgatct

SEQ ID NO: 42 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacccttgttaatacactctttccctacacgacgctcttccgatct

SEQ ID NO: 43 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacgttgatagtgacactctttccctacacgacgctcttccgatct

SEQ ID NO: 44 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacaccagcgacaacactctttccctacacgacgctcttccgatct

SEQ ID NO: 45 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacaccatacactgtacactctttccctacacgacgctcttccgatct

SEQ ID NO: 46 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacgtgtggcgctacactctttccctacacgacgctcttccgatct

SEQ ID NO: 47 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacatcacgaaggacactctttccctacacgacgctcttccgatct

SEQ ID NO: 48 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacaccggctctactacactctttccctacacgacgctcttccgatct

SEQ ID NO: 49 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacgaatgcacgaacactctttccctacacgacgctcttccgatct

SEQ ID NO: 50 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacaagactatagacactctttccctacacgacgctcttccgatct

SEQ ID NO: 51 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacactcggcagcaaacactctttccctacacgacgctcttccgatct

SEQ ID NO: 52 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacctaatgatggacactctttccctacacgacgctcttccgatct

SEQ ID NO: 53 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacggttgcctctacactctttccctacacgacgctcttccgatct

SEQ ID NO: 54 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacaccgcacatggcacactctttccctacacgacgctcttccgatct

SEQ ID NO: 55 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacggcctgtcctacactctttccctacacgacgctcttccgatct

SEQ ID NO: 56 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacctgtgttaggacactctttccctacacgacgctcttccgatct

SEQ ID NO: 57 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacactaaggaacgtacactctttccctacacgacgctcttccgatct

SEQ ID NO: 58 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacctaactgtaaacactctttccctacacgacgctcttccgatct

SEQ ID NO: 59 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacggcgagatggacactctttccctacacgacgctcttccgatct

SEQ ID NO: 60 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacaatagagcaaacactctttccctacacgacgctcttccgatct

SEQ ID NO: 61 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacactcaatccattacactctttccctacacgacgctcttccgatct

SEQ ID NO: 62 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacactcgtatgcggacactctttccctacacgacgctcttccgatct

SEQ ID NO: 63 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacactccgacctcgacactctttccctacacgacgctcttccgatct

SEQ ID NO: 64 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacaccttatggaatacactctttccctacacgacgctcttccgatct

SEQ ID NO: 65 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacgcttacggacacactctttccctacacgacgctcttccgatct

SEQ ID NO: 66 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacgaacatacggacactctttccctacacgacgctcttccgatct

SEQ ID NO: 67 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacgtcgattacaacactctttccctacacgacgctcttccgatct

SEQ ID NO: 68 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacactagccgtgacactctttccctacacgacgctcttccgatct

SEQ ID NO: 69 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacaagttggtgaacactctttccctacacgacgctcttccgatct

SEQ ID NO: 70 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacactggcaatattacactctttccctacacgacgctcttccgatct

SEQ ID NO: 71 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacgatcaccgcgacactctttccctacacgacgctcttccgatct

SEQ ID NO: 72 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacactaccatccgtacactctttccctacacgacgctcttccgatct

SEQ ID NO: 73 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacgctgtaggaaacactctttccctacacgacgctcttccgatct

SEQ ID NO: 74 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacaccgcactaatgacactctttccctacacgacgctcttccgatct

SEQ ID NO: 75 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacgacaactgaaacactctttccctacacgacgctcttccgatct

SEQ ID NO: 76 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacagtggtcaggacactctttccctacacgacgctcttccgatct

SEQ ID NO: 77 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacttctatggttacactctttccctacacgacgctcttccgatct

SEQ ID NO: 78 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacaatccggccaacactctttccctacacgacgctcttccgatct

SEQ ID NO: 79 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacccataaggttacactctttccctacacgacgctcttccgatct

SEQ ID NO: 80 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacacatctctaccaacactctttccctacacgacgctcttccgatct

SEQ ID NO: 81 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacaccggtggcgaaacactctttccctacacgacgctcttccgatct

SEQ ID NO: 82 - DNA, 72 bp, synthetic construct
  misc_feature 1..72: note = Primer
  modified_base 68..72: mod_base = OTHER; note = Phosphorothioate bonds
  source 1..72: mol_type = other DNA
aatgatacggcgaccaccgagatctacactaacaataggacactctttccctacacgacgctcttccgatct

(19) United States
(12) Patent Application Publication
(10) Pub. No.: US 2025/0243546 A1
(43) Pub. Date: Jul. 31, 2025

(54) HOST TRANSCRIPTOME FECAL BIOMARKERS FOR GASTROINTESTINAL INFLAMMATORY DISORDERS

(71) Applicants: YEDA RESEARCH AND DEVELOPMENT CO. LTD., Rehovot (IL); SHEBA IMPACT LTD., Ramat-Gan (IL); TRACELLS LTD., Kazrin (IL)

(72) Inventors: Shalev Saul ITZKOVITZ, Rehovot (IL); Stav DAN, Rehovot (IL); Shani BEN-MOSHE, Rehovot (IL); Adi EGOZI, Rehovot (IL); Keren Bahar HALPERN, Rehovot (IL); Bella UNGAR, Ramat-Gan (IL); Shomron Silan BEN-HORIN, Ramat-Efal (IL); Yotam HARNIK, Rehovot (IL); Avishag YEHUDA-COHEN, Jordan Valley (IL)

(73) Assignees: YEDA RESEARCH AND DEVELOPMENT CO. LTD., Rehovot (IL); SHEBA IMPACT LTD., Ramat-Gan (IL); TRACELLS LTD., Kazrin (IL)

(21) Appl. No.: 19/041,876

(22) Filed: Jan. 30, 2025

Related U.S. Application Data
(60) Provisional application No. 63/627,084, filed on Jan. 31, 2024.

(51) Int. Cl. C12Q 1/6883; C12Q 1/6806; C12Q 1/689

(52) U.S. Cl. CPC C12Q 1/6883; C12Q 1/689; C12Q 1/6806; C12Q 2600/158; C12Q 2600/16

(57) ABSTRACT

The invention provides assays and methods for analyzing inflammatory disorders of the gastrointestinal (GI) tract. Provided in embodiments of the invention are host transcriptome markers and classifiers amenable for assessing and monitoring the existence, severity and location of inflammation associated with inflammatory bowel disease (IBD), Crohn's disease (CD) and Ulcerative colitis (UC). Further provided are improved protocols for processing and analyzing fecal samples, providing superior non-invasive means for evaluating GI inflammation.

### RELATED APPLICATION

This application claims the benefit of priority of U.S. Provisional patent application No. 63/627,084 filed Jan. 31, 2024, the contents of which are incorporated herein by reference in their entirety.

### INCORPORATION BY REFERENCE OF A SEQUENCE LISTING XML

A Sequence Listing is provided herewith entitled “YEDA 0183 US SEQ.xml” created on Jan. 30, 2025 and having a size of 196,996 bytes. The contents of this electronic sequence listing are herein incorporated in their entirety.

### FIELD OF THE INVENTION

The invention provides diagnostic tools for analyzing inflammatory disorders of the gastrointestinal tract.

### BACKGROUND OF THE INVENTION

Inflammatory bowel diseases (IBDs) are autoinflammatory conditions associated with chronic inflammation of the gastrointestinal tract. Ulcerative colitis (UC) invariably involves the colon, whereas Crohn's disease (CD) can affect any colonic segment as well as the small intestine and even the upper gastrointestinal tract. Colonoscopy with biopsies currently is considered the gold standard for IBD diagnosis, stratification, and monitoring. Nevertheless, colonoscopy is an invasive procedure, heralding risks of perforation and infection. Development of less-invasive monitoring is an outstanding challenge. In addition, because only approximately 50% of IBD patients achieve endoscopic/histologic remission with the different currently available biological therapies, identification of noninvasive biomarkers that can assist in diagnosis, predict outcome, and guide therapy selection is needed.

Some of the inventors and coworkers have recently shown that host shed cell messenger RNAs (mRNAs) can be retrieved successfully from fecal washes and that transcriptomics of the host mRNA is associated significantly with endoscopic and even histologic healing in IBD patients with distal colitis (Ungar et al. Gut 2022; 71:1988-1997). WO 2023/002491, to some of the present inventors and coworkers, relates to methods of diagnosing gastric diseases and more particularly inflammatory bowel diseases, comprising analyzing the RNA expression level of human genes in a fecal RNA sample of the subject.

Another recent publication to the inventors and coworkers relates to the ability of distal fecal wash host transcriptomics to identify inflammation throughout the colon and terminal ileum (Dan et al. Cell Mol Gastroenterol Hepatol 2023; 16:1-15).

A recent study of biopsy transcriptomics in IBD patients by Powrie and colleagues (Friedrich et al. Nat Med 2021; 27:1970-1981) defined gene modules that were shown to correlate with either response or lack of response to biological therapies (anti-TNF and anti-integrin) and to corticosteroids.

Background art includes Cui et al., Digestive Diseases and Sciences (2021) 66:1488-1498; and U.S. patent application No. 20200308644.

There remains a long-felt need for additional diagnostic tools for providing early, accurate and minimally invasive means for characterizing parameters of gastrointestinal inflammation.

### SUMMARY OF THE INVENTION

The invention provides diagnostic tools for analyzing inflammatory disorders of the gastrointestinal (GI) tract. Embodiments of the invention relate to assays and methods amenable for diagnosing and prognosing patients afflicted with, or suspected of having, autoinflammatory conditions associated with chronic inflammation of the gastrointestinal tract such as inflammatory bowel diseases. More specifically, provided in embodiments of the invention are host transcriptome markers and classifiers amenable for assessing and monitoring the existence, severity and location of inflammation associated with inflammatory bowel disease (IBD), Crohn's disease (CD) and Ulcerative colitis (UC).

The invention, is based, in part, on the discovery of unique gene signatures based on measurements of RNA biomarkers in fecal samples, determined to be unexpectedly effective for evaluating GI inflammation. Surprisingly, as demonstrated herein, diagnostic markers and classifiers were developed, capable of providing improved discriminatory capacity. As disclosed and demonstrated herein, highly predictive models were constructed, capable of assessing the level and location of GI inflammation, as well as means for determining its presence in patients with diverse GI pathologies associated with IBD.

The invention is further based, in part, on development of improved protocols for processing and analyzing stool samples, providing superior non-invasive means for evaluating GI inflammation. In particular, it was found that inclusion of an additional step of substantially immediate freezing of the sample (within less than an hour of sample collection), facilitated improved analyses. More specifically, it was found that the modified protocol significantly improved the signal obtained when analyzing gene products in stool samples, such that a substantially larger portion of the stool samples collected were amenable for use in diagnosis.

Accordingly, disclosed herein are diagnostic assays and methods, useful for early diagnosis, prognosis, monitoring and management of GI inflammation and conditions associated therewith. In some embodiments, non-invasive and minimally-invasive assays and methods are provided. In other embodiments, diagnostic kits amenable for use with the methods of the invention are provided.

In one aspect, the invention provides a method of analyzing a fecal RNA sample, comprising:
i. providing a fecal RNA sample, by a method comprising:
   a) providing a frozen stool sample that had been stored at a temperature not higher than-20° C. within 1 hour of sample collection,
   b) recovering RNA from said sample to provide a fecal RNA sample, and
   c) subjecting said fecal RNA sample to selective depletion of microbial rRNA, and
ii. determining the level of at least one human gene product in the resulting fecal RNA sample.

In another embodiment, the method comprises:
i. providing a fecal RNA sample, by a method comprising:
   a) providing a stool sample of a subject afflicted with, or suspected of having, gastrointestinal (GI) inflammation,
   b) storing the stool sample at a temperature not higher than −20° C. within 1 hour of sample collection,
   c) recovering RNA from said sample to provide a fecal RNA sample, by thawing the frozen sample and isolating RNA from the thawed sample,
   d) subjecting said RNA sample to RNase H-based selective depletion of 5S, 16S and 23S rRNA of gram-positive and gram-negative bacteria,
   e) performing reverse transcription of the resulting depleted RNA, generating a library of gene products using RNA barcoding, and sequencing, and
ii. determining the level of at least one human gene product in the fecal RNA sample.

In another embodiment, the subject is diagnosed with, or suspected of having, an inflammatory bowel disease (IBD). In another embodiment, step (ii) comprises determining the levels in the sample of a plurality of the human gene products, thereby obtaining the transcriptomic signature of the sample with respect to the plurality of gene products. In another embodiment the method further comprises comparing the transcriptomic signature of said sample to a control transcriptomic signature by a supervised classification algorithm.

In another embodiment, the plurality of human gene products is selected from the group consisting of:
AC007192.1, ACSL1, ADGRG3, ALDOB, ALOX5AP, ALPL, AMN, ANP32E, AOC1, APBB1IP, APOA1, APOA4, APOB, APOC3, AQP9, ARHGAP26, ARHGAP30, ARRB2, BCL6, BID, CAPZA1, CASP5, CCL3, CCL3L3, CCL4, CCL4L2, CCR1, CD44, CD53, CD83, CDHR2, CDHR5, CLEC2B, CLEC4E, CLEC7A, CMTM2, CREB3L3, CREM, CSF3R, CXCL8, CXCR2, CYP3A4, CYTH1, CYTH4, DDX21, DEFA1B, DEFA3, DEFA5, DEFA6, DNAJA1, DPEP1, EIF3J, EPS8L1, ERICH1, EWSR1, FABP2, FABP6, FAM129A, FCER1G, FCGR2A, FCGR3A, FCGR3B, FNBP1, FPR1, FYB1, GOS2, GBP1, GBP4, GBP5, GDF5OS, GMFG, GNA11, GPR65; GUCA2A, GUCA2B, HCAR2, HCAR3, HNRNPA2B1, ICAM1, IFI16, IFIT2, IFIT3, IFITM2, IGSF6, IL1B, IL1R2, IL1RAP, IL1RN, ILF3, ITGAX, ITSN2, KCNJ15, KCNK6, KIAA0825, KIAA1109, LCP1, LCP2, LILRB3, LRRK2, LSP1, LYN, MAVS, MEP1A, METAP2, MNDA, MTRNR2L1, MTTP, NAMPT, NBN, NCF2, NCL, OLR1, OSM, PDE4B, PFKFB3, PHACTR1, PHIP, PLAU, PLCB2, PLCD3, PLEK, PPIF, PROK2, PTGS2, RAPGEF6, RCC1, REG1A, REG1B, RHOH, S100A4, S100A9, SAMSN1, SELENOP, SELL, SH3BP5, SI, SIPA1L2, SLC15A1, SLC2A3, SLC5A1, SMIM24, SNX10, SOCS3, SOD2, SP140, SRGN, SUPT6H, SYNE2, TANK, TET3, TLR2, TM4SF5, TMEM154, TNFAIP6, TRA2B, TREM1, UTRN, VNN2, WDR66, YTHDC1, ZEB2, ZFC3H1, ZNF267, and ZNF511-PRAP1.

In another embodiment, said plurality of human gene products is selected from the group consisting of:
a. TET3, RAPGEF6, RCC1, IL1R2, WDR66, ANP32E, EPS8L1, ALPL, ILF3, ERICH1, PLCD3, MAVS, SIPA1L2, ARHGAP30, FNBP1, NCL, EWSR1, SP140, DDX21, KCNK6, KIAA0825, SUPT6H, CYTH1, EIF3J, ARHGAP26, MTRNR2L1, PLCB2, UTRN, METAP2, and GDF5OS (Group A gene products);
b. REG1A, FABP6, REG1B, APOB, ALDOB, ZNF511-PRAP1, SI, CYP3A4, APOA1, DEFA6, APOA4, DPEP1, DEFA5, APOC3, CREB3L3, AMN, SLC15A1, GUCA2A, SMIM24, MTTP, SLC5A1, FABP2, MEP1A, TM4SF5, GUCA2B, AOC1, CDHR5, CDHR2, SELENOP, and GNA11 (Group B gene products);
c. AC007192.1, ACSL1, ALOX5AP, AQP9, BCL6, CCL4, CD44, CSF3R, CXCL8, DEFA1B, FAM129A, FCGR2A, FCGR3B, FPR1, FYB1, GOS2, GBP1, GMFG, HCAR2, HCAR3, ICAM1, IFI16, IFITM2, IL1B, IL1RN, ITGAX, LCP1, LCP2, LILRB3, LYN, MNDA, NAMPT, NCF2, OSM, PDE4B, PFKFB3, PLEK, PPIF, PROK2, PTGS2, S100A4, S100A9, SAMSN1, SLC2A3, SOCS3, SOD2, SRGN, TNFAIP6, TREM1, and ZNF267 (Group C gene products);
d. CAPZA1, CASP5, DNAJA1, HNRNPA2B1, ITSN2, KIAA1109, PHIP, SYNE2, TANK, TRA2B, YTHDC1, and ZFC3H1 (Group D gene products);
e. GBP5, DEFA3, DEFA1B, FCGR3A, CLEC2B, SH3BP5, CD53, CLEC7A, PLAU, IL1RAP, CCR1, FCER1G, CLEC4E, TLR2, LSP1, ADGRG3, CD83, TREM1, CMTM2, RHOH, APBB1IP, LRRK2, BID, KCNJ15, IFIT2, PLEK, PDE4B, HCAR3, ITGAX, SELL, MNDA, HCAR2, ICAM1, FCGR2A, CCL4L2, CCL3L3, GBP1, CSF3R, CD44, PROK2, SOCS3, GMFG, S100A4, TNFAIP6, SNX10, NBN, OSM, SOD2, IFI16, FYB1, AC007192.1, FCGR3B, IL1B, CYTH4, IL1RN, OLR1, VNN2, CCL3, CCL4, IFIT3, CREM, ZEB2, ALOX5AP, CXCL8, LCP2, IGSF6, CXCR2, ZNF267, GBP4, LCP1, PHACTR1, ARRB2, TMEM154, BCL6, and GPR65 (Group E gene products); and/or
f. GBP5, DEFA3, DEFA1B, FCGR3A, CLEC2B, SH3BP5, CD53, CLEC7A, PLAU, IL1RAP, CCR1, FCER1G, CLEC4E, TLR2, LSP1, ADGRG3, CD83, TREM1, CMTM2, RHOH, APBB1IP, LRRK2, BID, KCNJ15, IFIT2, PLEK, PDE4B, HCAR3, ITGAX, SELL, MNDA, HCAR2, ICAM1, FCGR2A, and CCL4L2 (Group F gene products).

In another embodiment, said plurality of human gene products is selected from the group consisting of:
a. RNASEK, RNASEK-C17orf49, MIDN, HLA-A, B2M, HLA-B, HLA-C, CAP1, PFN1, ACTB, MXD1, SAT1, LITAF, NFKBIA, S100A9, S100A8, EIF1, FTL, and FTH1 (Group G gene products),
b. AC138811.2, AQP9, ARPC2, ARPC5, BASP1, BCL2A1, BRI3, BTG2, CALM2, CCL4, CDKN1A, CEACAM1, CEBPB, CXCL8, EGR1, ETS2, FOS, FPR1, FTL, GOS2, GABARAP, GLUL, GNB2, HCAR3, HLA-C, HLA-E, ICAM1, IFITM1, IFITM2, IL1B, IL1RN, IRF1, ISG20, ITM2B, IVNS1ABP, KDM6B, KLF6, LITAF, MARCKS, MCL1, MXD1, NAMPT, NFKBIA, OSM, PFN1, PLAUR, PLEK, PNRC1, PPIF, PROK2, PTP4A1, S100A11, S100A8, S100A9, SAT1, SDCBP, SLC25A37, SOCS3, SOD2, SRGN, TAF10, TNFAIP3, TPM4, TXNIP, TYMP, UBE2B, VASP, WDR83OS, ZFP36, and ZFP36L1 (Group H gene products),
c. CCL4, CXCL8, HCAR3, ICAM1, IL1B, IL1RN, OSM, PLEK, PROK2, SOCS3, SOD2 (Group I gene products), and/or
d. ABHD17C, AC005943.1, ACTN4, ALDOA, AP000350.4, AP000721.1, AP003419.1, C17orf49, CA4, CDHR5, CFL1, CKB, COX7A2, COX8A, CRIP1, CST3, CTNND1, DBNDD2, DYNLRB1, EGLN1, EIF4G2, EPCAM, FABP1, FCGBP, FXYD3, GUCA2A, GUCA2B, IFI27, KRT8, LGALS4, LYPD8, MAL2, MGLL, MIF, MISP, MUC12, MUC2, MYL12B, OST4, P2RX5-TAX1BP3, PDLIM1, PHGR1, PIGR, PLAC8, POLD4, PPDPF, RHOC, S100A16, SDCBP2, SERINC2, SFN, SH3BGRL3, SMIM22, SPINT2, SRI, STK24, SYS1-DBNDD2, TAX1BP3, TFF1, TFF3, TMEM54, TMPRSS2, TMSB10, TRIM31, UBA52, UBB, UQCR11, ZG16 (Group J gene products).

In another embodiment, the outcome of the comparison is indicative of the location of GI inflammation in said subject, and said plurality of human gene products is as set forth in Group A and/or B. In another embodiment, the outcome of the comparison is indicative of the severity of GI inflammation in said subject, and said plurality of human gene products is as set forth in Group C and/or D. In another embodiment the outcome of the comparison is indicative of the presence or absence of GI inflammation in said subject, and said plurality of human gene products is as set forth in Group E, F, G, H, I and/or J. In another embodiment the plurality of gene products further comprises at least one additional gene product selected from Table 4.

In another embodiment of the methods of the invention, the subject is afflicted with, or suspected of having, ulcerative colitis (UC), or wherein the subject is afflicted with, or suspected of having, Crohn's disease (CD). In another embodiment of the methods of the invention, determining the levels of each gene product in the sample comprises determining the Unique Molecular Identifier (UMI) counts for each gene product. In another embodiment determining the levels of each gene product in the sample further comprises normalizing the level of each UMI in said sample to the levels of other UMIs in said sample. In another embodiment of the methods of the invention, step (ii) comprises determining the levels in the sample of a plurality of the human gene products, thereby obtaining the transcriptomic signature of the sample with respect to the plurality of gene products wherein said plurality of gene products is selected from Table 1 or from Table 4. In another embodiment of the methods of the invention, the stool sample is stored at a temperature not higher than-20° C. within 1 hour of sample collection in the presence of an RNA preservation reagent comprising ammonium sulfate and ethylenediaminetetraacetic acid (EDTA) and in the absence of a cryoprotectant.

In another aspect, there is provided a method of analyzing a fecal RNA sample, comprising:
i. providing a fecal RNA sample of a subject afflicted with, or suspected of having, GI inflammation,
ii. determining the levels in the sample of a plurality of human gene products, thereby obtaining the transcriptomic signature of the sample with respect to the plurality of gene products, and
iii. comparing the transcriptomic signature of said sample to a control transcriptomic signature by a supervised classification algorithm,
   wherein said plurality of gene products is selected from the group consisting of: Group A gene products, Group B gene products, Group C gene products, Group D gene products, Group E gene products, Group F gene products, Group G gene products, Group H gene products, Group I gene products), and/or Group J gene products.

In another embodiment, providing said fecal RNA sample comprises:
a) providing a frozen stool sample that had been stored at a temperature not higher than −20° C. within 1 hour of sample collection,
b) recovering RNA from said sample to provide a fecal RNA sample, and
c) subjecting said fecal RNA sample to selective depletion of microbial rRNA.

In another embodiment, providing said fecal RNA sample comprises:
a) providing a stool sample of a subject afflicted with, or suspected of having, GI inflammation,
b) storing the stool sample at a temperature not higher than −20° C. within 1 hour of sample collection,
c) recovering RNA from said sample to provide a fecal RNA sample, by thawing the frozen sample and isolating RNA from the thawed sample,
d) subjecting said RNA sample to RNase H-based selective depletion of 5S, 16S and 23S rRNA of gram-positive and gram-negative bacteria,
e) performing reverse transcription of the resulting depleted RNA, and
f) generating a library of gene products using RNA barcoding and sequencing the generated library.

In another embodiment, the stool sample had been obtained from a subject afflicted with, or suspected of having, an IBD.

In another embodiment, the methods of the invention include: providing a frozen stool sample that has been stored at a temperature not higher than −20° C. within 1 hour of sample collection, recovering RNA from said sample to provide a fecal RNA sample, selectively depleting microbial rRNA from the fecal RNA sample, and analyzing the depleted fecal RNA sample to determine one or more human gene products therein. In another embodiment the stool sample had been obtained from a subject afflicted with, or suspected of having, GI inflammation. In another embodiment, there is provided a method of determining one or more human gene products from a stool sample, comprising:
a) providing a frozen stool sample that has been stored at a temperature not higher than −20° C. within 1 hour of sample collection,
b) recovering RNA from said sample to provide a fecal RNA sample,
c) selectively depleting microbial rRNA from the fecal RNA sample, and
d) analyzing the depleted fecal RNA sample of step c) to determine one or more human gene products therein,
   wherein step (d) comprises determining the levels in the sample of a plurality of the human gene products selected from Table 1 or from Table 4, thereby obtaining the transcriptomic signature of the sample with respect to the plurality of gene products.

In another aspect, there is provided a diagnostic kit, comprising means for specifically determining and quantifying the levels of a plurality of human gene products in a fecal RNA sample, wherein the plurality of human gene products comprises or consists of: Group A gene products, Group B gene products, Group C gene products, Group D gene products, Group E gene products, Group F gene products, Group G gene products, Group H gene products, Group I gene products), and/or Group J gene products.

In another embodiment said plurality of gene products comprises or consists of Group A, B, C, D, E and/or F gene products. In another embodiment the means comprise quantitative polymerase chain reaction (qPCR) primers directed to said plurality of human gene products, and the kit optionally further comprises means for providing a fecal RNA sample and/or means for comparing the levels of said plurality of human gene products in the sample to their levels in a control fecal RNA sample.

In another aspect, there is provided a method of determining the location of GI inflammation, comprising:
i. providing a fecal RNA sample of a subject afflicted with, or suspected of having, GI inflammation,
ii. determining the levels in the sample of a plurality of human gene products, thereby obtaining the transcriptomic signature of the sample with respect to the plurality of gene products, and
iii. comparing the transcriptomic signature of said sample to a control transcriptomic signature by a supervised classification algorithm,
   wherein said plurality of gene products is selected from the group consisting of Group A gene products, and/or Group B gene products, wherein the outcome of the comparison is indicative of the location of GI inflammation in said subject, and wherein the method further comprises providing said subject with a treatment suitable for the determined location of GI inflammation in said subject, comprising:
Metronidazole, Budesonide, Ciprofloxacin, Metronidazole, Certolizumab pegol (CZP),
Infliximab, Ustekinumab or anti-TNF alpha antibodies if said GI inflammation is determined to be located in the colon, and
Enteral Nutrition or Adalimumab if said GI inflammation is determined to be located in the terminal ileum.

In another embodiment, said subject is suspected of having colonic GI inflammation or terminal ileal GI inflammation. In another embodiment said subject is suspected of having colonic CD or terminal ileal CD. In another embodiment step (ii) comprises determining the transcriptomic signatures of the sample with respect to the first plurality of gene products of Group A and the second plurality of gene products of Group B. In another embodiment the control transcriptomic signature comprises at least one of: a control transcriptomic signature with respect to the plurality of gene products corresponding to colonic CD that does not involve the terminal ileum, and a control transcriptomic signature with respect to the plurality of gene products corresponding to terminal ileal CD. In another embodiment the method comprises comparing said transcriptomic signatures to a first control transcriptomic signature corresponding to colonic CD that does not involve the terminal ileum and to a second control transcriptomic signature corresponding to terminal ileal CD.

In another embodiment said transcriptomic signature reflects the collective levels of said first plurality of gene products in said sample as compared to the collective levels of said second plurality of gene products in said sample, and wherein
the higher the enhancement of the collective levels of said first plurality of gene products in said sample as compared to the collective levels of said second plurality of gene products in said sample, the higher the probability that said subject is afflicted with colonic CD that does not involve the terminal ileum, and/or
the higher the enhancement of the collective levels of said second plurality of gene products in said sample as compared to the collective levels of first plurality of gene products in said sample, the higher the probability that said subject is afflicted with terminal ileal CD.

In another embodiment, a transcriptomic signature characterized by significant enhancement of the collective levels of said first plurality of gene products in said sample as compared to a control transcriptomic signature corresponding to terminal ileal CD indicates that said subject is afflicted with colonic CD, and/or a transcriptomic signature characterized by significant enhancement of the collective levels of said second plurality of gene products in said sample as compared to a control transcriptomic signature corresponding to colonic CD that does not involve the terminal ileum indicates that said subject is afflicted with terminal ileal CD.

In another embodiment said sample is a fecal wash sample. In a particular embodiment said sample is a distal fecal wash sample. In another embodiment said sample is a stool sample. In a particular embodiment providing said fecal RNA sample comprises: providing a frozen stool sample that had been stored at a temperature not higher than −20° C. within 1 hour of sample collection, recovering RNA from said sample to provide a fecal RNA sample, and subjecting said fecal RNA sample to selective depletion of microbial rRNA. In another embodiment, providing said fecal RNA sample comprises: providing a stool sample of a subject afflicted with, or suspected of having, GI inflammation, storing the stool sample at a temperature not higher than −20° C. within 1 hour of sample collection, recovering RNA from said sample to provide a fecal RNA sample, by thawing the frozen sample and isolating RNA from the thawed sample, subjecting said RNA sample to RNase H-based selective depletion of 5S, 16S and 23S rRNA of gram-positive and gram-negative bacteria, performing reverse transcription of the resulting depleted RNA, and generating a library of gene products using RNA barcoding, and sequencing the generated library.

In another aspect, there is provided a method of determining the location of GI inflammation, comprising:
i. providing a fecal RNA sample of a subject afflicted with, or suspected of having, GI inflammation,
ii. determining the levels in the sample of a plurality of human gene products, thereby obtaining the transcriptomic signature of the sample with respect to the plurality of gene products, and
iii. comparing the transcriptomic signature of said sample to a control transcriptomic signature by a supervised classification algorithm,
   wherein said plurality of gene products is selected from Table 4, wherein the outcome of the comparison is indicative of the location of GI inflammation in said subject, and wherein the method further comprises providing said subject with a treatment suitable for the determined location of GI inflammation in said subject, comprising:
Metronidazole, Budesonide, Ciprofloxacin, Metronidazole, Certolizumab pegol (CZP),
Infliximab, Ustekinumab or anti-TNF alpha antibodies if said GI inflammation is determined to be located in the colon, and
Enteral Nutrition or Adalimumab if said GI inflammation is determined to be located in the terminal ileum.

In another aspect, there is provided a method of determining the severity of GI inflammation, comprising:
i. providing a fecal RNA sample of a subject afflicted with, or suspected of having, GI inflammation,
ii. determining the levels in the sample of a plurality of human gene products, thereby obtaining the transcriptomic signature of the sample with respect to the plurality of gene products, and
iii. comparing the transcriptomic signature of said sample to a control transcriptomic signature by a supervised classification algorithm,
   wherein said plurality of gene products is selected from the group consisting of Group C gene products and Group D gene products,
   wherein the outcome of the comparison is indicative of the severity of GI inflammation in said subject, and wherein the method further comprises providing said subject with a treatment suitable for the determined severity of GI inflammation in said subject, comprising:
topical mesalamine, oral mesalamine, topical steroids and/or Budesonide if the determined severity is mild,
systemic and topical steroids, oral mesalamine, ozanimod, upadacitinib, tofacitinib, risankizumab, ustekinumab, vedolizumab, azathioprine (AZA), Thiopurines and/or Budesonide if the determined severity is moderate, and
intravenous steroids, infliximab (IFX) and AZA, cyclosporin and AZA, IFX, adalimumab, golimumab and AZA, surgery and/or natalizumab if the determined severity is severe.

In another embodiment, step (ii) comprises determining the transcriptomic signature of the sample with respect to the first plurality of gene products and the second plurality of gene products. In another embodiment, the control transcriptomic signature comprises control transcriptomic signature with respect to the plurality of gene products corresponding to at least one of: a healthy control, inactive GI inflammation, mild GI inflammation, moderate GI inflammation, and severe GI inflammation. In another embodiment, the control transcriptomic signature comprises control transcriptomic signature with respect to the plurality of gene products corresponding to at least one of: a healthy control, GI inflammation characterized by an endoscopic score of at least 2, and GI inflammation characterized by an endoscopic severity lower than 2, wherein said endoscopic severity is determined according to the Mayo and/or Simple Endoscopic Score (SESCD) scores. In another embodiment, said transcriptomic signature reflects the collective levels of said first plurality of gene products in said sample as compared to the collective levels of said second plurality of gene products in said sample, and wherein the higher the enhancement of said collective levels of said first plurality of gene products in said sample as compared to said collective levels of said second plurality of gene products in said sample, the higher the predicted severity of GI inflammation in said subject.

In another embodiment said sample is a fecal wash sample. In a particular embodiment said sample is a distal fecal wash sample. In another embodiment said sample is a stool sample. In a particular embodiment providing said fecal RNA sample comprises: providing a frozen stool sample that had been stored at a temperature not higher than −20° C. within 1 hour of sample collection, recovering RNA from said sample to provide a fecal RNA sample, and subjecting said fecal RNA sample to selective depletion of microbial rRNA. In another embodiment, providing said fecal RNA sample comprises: providing a stool sample of a subject afflicted with, or suspected of having, GI inflammation, storing the stool sample at a temperature not higher than-20° C. within 1 hour of sample collection, recovering RNA from said sample to provide a fecal RNA sample, by thawing the frozen sample and isolating RNA from the thawed sample, subjecting said RNA sample to RNase H-based selective depletion of 5S, 16S and 23S rRNA of gram-positive and gram-negative bacteria, performing reverse transcription of the resulting depleted RNA, and generating a library of gene products using RNA barcoding, and sequencing the generated library.

In another aspect, there is provided a method of identifying GI inflammation, comprising:
i. providing a fecal RNA sample of a subject afflicted with, or suspected of having, GI inflammation,
ii. determining the levels in the sample of a plurality of human gene products, thereby obtaining the transcriptomic signature of the sample with respect to the plurality of gene products, and
iii. comparing the transcriptomic signature of said sample to a control transcriptomic signature by a supervised classification algorithm,
   wherein said plurality of gene products is selected from the group consisting of Group E gene products, Group F gene products, Group G gene products, Group H gene products and/or Group I gene products,
   wherein the outcome of the comparison is indicative of the presence or absence of GI inflammation in said subject, and wherein the method further comprises providing said subject with a suitable treatment selected from the group consisting of: corticosteroids, prednisone, prednisolone, methylprednisolone, 5-ASA drugs, olsalazine, sulfasalazine, immunomodulators, azathioprine, mercaptopurine, TNF-alpha inhibitors, certolizumab; natalizumab; ustekinumab, vedolizumab and surgery.

In another embodiment, said plurality of gene products further comprises (or is selected from the group consisting of) Group J gene products. In another embodiment the control transcriptomic signature corresponds to a healthy control, and wherein a transcriptomic signature characterized by significant enhancement of the collective levels of said plurality of gene products in said sample as compared to said healthy control indicates that said subject is afflicted with GI inflammation. In another embodiment the control transcriptomic signature corresponds to a healthy control, and wherein a transcriptomic signature characterized by significant enhancement of the collective levels of gene products selected from Group E, F, G, H and/or I, and by a significant reduction of the collective levels of gene products selected from Group J, in said sample as compared to said healthy control, indicates that said subject is afflicted with GI inflammation.

In another embodiment said sample is a fecal wash sample. In a particular embodiment said sample is a distal fecal wash sample. In another embodiment said sample is a stool sample. In a particular embodiment providing said fecal RNA sample comprises: providing a frozen stool sample that had been stored at a temperature not higher than −20° C. within 1 hour of sample collection, recovering RNA from said sample to provide a fecal RNA sample, and subjecting said fecal RNA sample to selective depletion of microbial rRNA. In another embodiment, providing said fecal RNA sample comprises: providing a stool sample of a subject afflicted with, or suspected of having, GI inflammation, storing the stool sample at a temperature not higher than −20° C. within 1 hour of sample collection, recovering RNA from said sample to provide a fecal RNA sample, by thawing the frozen sample and isolating RNA from the thawed sample, subjecting said RNA sample to RNase H-based selective depletion of 5S, 16S and 23S rRNA of gram-positive and gram-negative bacteria, performing reverse transcription of the resulting depleted RNA, and generating a library of gene products using RNA barcoding, and sequencing the generated library.

Other objects, features and advantages of the present invention will become clear from the following description and drawings.

### BRIEF DESCRIPTION OF THE DRAWINGS

FIG. 1. Heatmap showing the expression of the segmentally-informative genes in biopsies and fecal washes. Each row is a gene, each column is a sample type, biopsies are from Crohn's patients with active inflammation, either in the colon (first column) or terminal ileum (TI, second column). Washes are all from the distal colon and originate from either patients with inflammation that includes the distal colon (third column) or TI inflammation with no distal colonic involvement (fourth column). Columns 1÷2 are normalized to their maximum across these two biopsy locations, columns 3÷4 normalized to their maximum across these two wash types.

FIGS. 2A to 2C. Violin plots showing the expression of genes (UMI counts divided by the sum of UMI counts of all genes in each sample) that are differentially expressed between distal fecal washes of patients with MAYO/SESCD score 2 or higher and MAYO/SESCD score lower than 2. FIG. 2A shows the expression of: AC007192.1, ACSL1, ALOX5AP, DEFA1B, FAM129A, FCGR2A, HCAR2, HCAR3, ICAM1, LCP2, LILRB3, LYN, PLEK, PPIF, PROK2, SOD2, SRGN, TNFAIP6; FIG. 2B shows the expression of: AQP9, BCL6, FCGR3B, FPR1, CCL4, IFI16, IFITM2, FYB1, MNDA, NAMPT, IL1B, PTGS2, S100A4, NCF2, TREM1, ZNF267, S100A9. FIG. 2C shows the expression of. CD44, CSFR3, CXCL8, GOS2, GBP1, GMFG, IL1RN, ITGAX, LCP1, OSM, PDE4B, PFKFB3, SAMSN1, SLC2A3, and SOCS3.

FIGS. 3A and 3B. Violin plots showing the expression of genes (UMI counts divided by the sum of UMI counts of all genes in each sample) that are differentially expressed between distal fecal washes of patients with MAYO/SESCD score 0 or 1 and patients with no inflammation. FIG. 3A shows the expression of: CAPZA1, CASP5, ITSN2, KIAA1109, TANK, TRA2B; FIG. 3B shows the expression of: DNAJA1, HNRNPA2B1, PHIP, SYNE2, YTHDC1, and ZFC3H1.

FIG. 4. Violin plots of the severity score over samples of different endoscopic severity classes. Horizontal lines indicate the chosen thresholds for the classification of samples into no inflammation (below 0.4), endoscopic severity 0/1 (score between 0.4 and 0.75) and endoscopic severity 2/3 (score above 0.75).

FIGS. 5A and 5B. Improvement of new stool storage protocol compared to old storage protocol. FIG. 5A-fraction of samples that passed the minimum UMI count sufficient to enter the analysis. FIG. 5B-median UMI count of all samples that entered the analysis.

FIGS. 6A to 6D. Classification using a 75-marker panel based on stool cohort of 25 patients and fecal washes cohort of 84 patient. FIG. 6A-Inflammation signature score in distal washes with no inflammation (ctrl) and with inflammation (Ibd). FIG. 6B-Inflammation signature score in stools from non-IBD patients (ctrl) and IBD patients (ibd). Signature scores in 6A-6B were obtained by summing the max-normalized UMI-sum normalized expression of 75 signature genes with the highest fold change between ibd and ctrl. FIGS. 6C and 6D-Receiver Operating Curve analyses of the data in FIGS. 6A and 6B, respectively.

FIGS. 7A to 7D. Classification using a 35-marker panel based on stool cohort of 25 patients and fecal washes cohort of 84 patients. FIG. 7A-Inflammation signature score in distal washes with no inflammation (ctrl) and with inflammation (ibd). FIG. 7B-Inflammation signature score in stools from non-IBD patients (ctrl) and IBD patients (ibd). Signature scores in FIGS. 7A-7B were obtained by summing the max-normalized UMI-sum normalized expression of 35 signature genes with the highest fold change between ibd and ctrl. FIGS. 7C and 7D—Receiver Operating Curve analyses of the data in FIGS. 7A and 7B, respectively.

FIGS. 8A to 8D. Host transcriptomics of fecal washes from different intestinal segments captures information that is distinct from same—segment biopsy transcriptomics. FIG. 8A—Experimental layout. FIG. 8B—Clustergram of transcriptomes of biopsies and fecal washes, color-coded by intestinal segment source and inflammation status. Dark—high expression, light—low expression. Representative gene names are shown on the right. FIG. 8C—Principal component analysis (PCA) of biopsies (dark) and fecal washes (light). Black circles highlight inflamed samples. Numbers in parentheses show percent of explained variance by each PC. FIG. 8D-Spearman correlations between the transcriptomes of pairs of either biopsies or fecal washes that are both annotated as inflamed and mixed pairs (one annotated as inflamed and the other not). Biopsies are considered inflamed if they exhibit histological inflammation, fecal washes are considered inflamed if at least one segmental biopsy from the patient showed inflammation. TI—terminal ileum, P—proximal colon, D—distal colon.

FIGS. 9A to 9F. Host transcriptomics of distal fecal washes captures active inflammation in ileal or proximal colonic Crohn's disease. FIG. 9A—Clustergram of distal fecal washes from UC and controls, color-coded by disease and segment of inflammation. FIG. 9B—Principal component analysis (PCA) of UC and control distal fecal washes. Numbers in parentheses show percent of explained variance by each PC. FIG. 9C—Clustergram of distal fecal washes from CD and controls, color-coded by disease and segment of inflammation. FIG. 9D-Principal component analysis (PCA) of CD and control distal fecal washes. Numbers in parentheses show percent of explained variance by each PC. Legend (from top to bottom): —no inflammation, inflammation includes the distal colon (left), inflammation in the proximal colon (right) or proximal colon+terminal ileum, inflammation only in the terminal ileum. In FIGS. 9A and 9C dark—high expression, light—low expression, representative gene names are shown on the right. framed branches highlight fecal washes enriched in inflamed samples. FIG. 9E—Differential gene expression (DGE) of inflamed and non-inflamed distal fecal washes from CD and controls. Gray—significantly different genes (fold change above 2 and q-value below 0.1). FIG. 9F—Receiver operating characteristic (ROC) curve of the classification performance of inflammation status based on distal fecal washes of controls and UC patients, distal fecal washes of controls and CD patients that does not involve the distal colon and distal biopsies of controls vs. UC and CD patients. Biopsies are considered inflamed if they exhibit histological inflammation, fecal washes are considered inflamed if at least one segmental biopsy from the patient showed inflammation. UC-ulcerative colitis, CT-control, CD-Crohn's disease, inf—inflamed, TI-terminal ileum.

FIGS. 10A to 10E. Transcriptomics of distal fecal washes contain information referring to the inflamed intestinal segment. FIG. 10A-Scatter plot of the ratios of expression between inflamed samples of CD patients with distal colon inflammation and ones with only TI inflammation. X axis shows the ratios in the segmental biopsies, Y axis shows the ratios in distal fecal washes. FIGS. 10B to 10E-Violin plots of the expression of representative genes highlighted in FIG. 10A, as follows: FIG. 10B—REG1A, FIG. 10C—APOC3, FIG. 10D—SULF2, FIG. 10E—SUPT6H. Biopsies are considered inflamed if they exhibit histological inflammation, fecal washes are considered inflamed if at least one segmental biopsy from the patient showed inflammation. TI—terminal ileum, inf—inflamed.

FIGS. 11A to 11D. Distal fecal washes contain modules of co-expressed immune, stromal and epithelial genes associated with inflammation severity. FIGS. 11A to 11C—Clustergram of epithelial genes (FIG. 11A), stromal genes (FIG. 11B) and immune genes (FIG. 11C). Dark—high expression, light—low expression. Modules are based on the gene hierarchical clustering, relevant branches colored accordingly. Representative module genes are shown on the right. Colorbars annotate disease and inflammation status. Framed branches highlight a branch of fecal washes enriched in inflamed samples. FIGS. 11D-Clustergram of the module scores for distal fecal wash samples demonstrate co-expression of the Str3, Imm3 and Epi1 modules. dark—high expression, light—low expression. Colorbars annotate disease and inflammation severity score. Heatmap on the right shows the median of module scores over the discrete inflammation severity score classes. Each row normalized to the maximum across severity classes. Fecal washes are considered inflamed if at least one segmental biopsy from the patient showed inflammation. UC—ulcerative colitis, CD—Crohn's disease, Epi—epithelial, Str—stromal, Imm—immune, inf—inflamed, Non-inf—non inflamed.

FIGS. 12A to 12E. Modules of co-expressed genes that correlate with response to biological therapy carry information on inflammation severity in fecal washes. FIGS. 12A to 12C—Summed expression of previously identified modules that showed correlation with response to anti-TNF/anti-integrin therapies. FIG. 12A—Distal fecal washes, framed branch highlights a branch of fecal washes enriched in inflamed samples, elevated in modules M1, M3, M4, M5, M6. FIG. 12B—All biopsies. Colorbars in (FIGS. 12A-12B) annotate disease and inflammation status. FIG. 12C—Only inflamed distal fecal washes. Colorbars annotate disease and inflammation severity. Bold black branch highlights fecal washes of patients with severe inflammation. FIG. 12D—Scores of representative modules correlating with non-response to anti-TNF/anti-integrin therapies, showing more significant differences in expression between inflamed and non-inflamed distal fecal washes compared to biopsies for M1, M4, M5 but not M2. A pseudonumber (2.4163e-05) was added to the module scores before applying the log10 transform. FIG. 12E-Higher statistical significance in the differences in module expression between inflamed and non-inflamed samples in distal fecal washes compared to biopsies. Shown are −log10 (qvalues). Biopsies are considered inflamed if they exhibit histological inflammation, fecal washes are considered inflamed if at least one segmental biopsy from the patient showed inflammation. UC—ulcerative colitis, CD—Crohn's disease, inf—inflamed, non-inf—non inflamed.

FIGS. 13A and 13B. Illustration of samples classification and differences in gene expression between biopsies and fecal washes. FIG. 13A-Heatmap showing the source of the patients' samples in the study cohort, with reference to the previous dataset. FIG. 13B—Differential gene expression (DGE) between fecal washes and biopsies. Gray—significantly different genes (fold change above 2 and q-value below 0.1). Shown are the names of representative differentially-expressed genes. UC—ulcerative colitis, CD—Crohn's disease, TI—terminal ileum.

FIGS. 14A and 14B. Computational deconvolution shows differences in cell composition between biopsies and fecal washes. FIG. 14A—Clustergram of inferred cell proportions. Table was standardized to Z-scores. Framed branch highlights fecal washes enriched in inflamed samples. Colorbars annotate samples by sample type, disease and inflammation status. FIG. 14B—Violin plots of the inferred proportions of distinct cell types in biopsies (top) and fecal washes (bottom). Non-inflamed samples include both controls and non-inflamed IBD patients. Biopsies are considered inflamed if they exhibit histological inflammation, fecal washes are considered inflamed if at least one segmental biopsy from the patient showed inflammation. UC—ulcerative colitis, CD—Crohn's disease.

FIG. 15. Module scores of co-expressed immune, stromal and epithelial genes exhibit trends that correlate with inflammation severity. Shown are the module scores of each of the ten modules binned according to classes of inflammation severity (Table 6). The classes were binned by the percentile of the pre-defined inflammation severity score (controls were assigned severity score 0). Shown are Spearman correlation values between the module scores and severity scores of distal fecal washes from patients with inflammation and controls. Fecal washes are considered inflamed if at least one segmental biopsy from the patient showed inflammation. Epi—epithelial, Str—stromal, Imm—immune, inf—inflamed, Non-inf—non inflamed.

FIGS. 16A and 16B. Fecal wash host transcriptomics correlates with endoscopic severity score significantly more than calprotectin levels. FIG. 16A-Module scores of co-expressed immune, stromal and epithelial gene modules are in correlation with endoscopic severity score. Module scores are binned according to classes of endoscopic severity scores (Table 6, controls were assigned endoscopic score 0). Shown are Spearman correlation values between the module scores and endoscopic severity scores of distal fecal washes from patients with inflammation and controls. Top and bottom panels highlight gene modules which significantly decrease and increase with endoscopic inflammation severity, respectively (p-value<0.05). FIG. 16B—Correlation between calprotectin levels and endoscopic scores of distal fecal washes from patients with inflammation (Table 6). Fecal washes are considered inflamed if at least one segmental biopsy from the patient showed inflammation. Epi—epithelial, Str—stromal, Imm—immune.

FIGS. 17A and 17B. Evaluation of human transcriptomic signal in stool samples collected using home kits. Shown are 15 stool samples that were split into two and stored under two conditions: −20° ° C. storage for 1 day (“1 day freezer”) and −20° C. storage for 5 days (“5 day freezer”). Samples were then processed using a human fecal transcriptomics protocol. FIG. 17A—number of human genes detected (“# of Genes”). FIG. 17B—the percentage of reads mapped to the human genome (“% Human reads”).

FIG. 18. Classifier for active inflammation using stool samples. Shown are fecal RNA inflammation genes score for IBD patients experiencing flare-ups (n=21) and for healthy controls or IBD patients in remission (n=22), as determined by endoscopic evaluation. Violin plots depict the distribution of RNA scores with individual data points displayed. The white circle marks the median score, while the thick black bar represents the interquartile range. The dashed line indicates a threshold score of 542.73, which differentiates between flare and remission states with 90% sensitivity and specificity.

### DETAILED DESCRIPTION OF THE INVENTION

The invention provides assays and methods for analyzing inflammatory disorders of the gastrointestinal (GI) tract. Provided in embodiments of the invention are host transcriptome markers and classifiers amenable for assessing and monitoring the existence, severity and location of inflammation associated with inflammatory bowel disease (IBD), Crohn's disease (CD) and Ulcerative colitis (UC). Further provided are improved protocols for processing and analyzing fecal samples, providing superior non-invasive means for evaluating GI inflammation.

According to embodiments of the invention, provided are methods including providing a fecal sample, in particular a fecal RNA sample, and determining the level of at least one human gene product in the fecal RNA sample. Typically, the methods of the invention comprise determining the levels in the sample of a plurality of human gene products as disclosed herein, thereby obtaining the transcriptomic signature of the sample with respect to the plurality of gene products. In some embodiments, the methods of the invention further comprise a step of comparing the transcriptomic signature of said sample to a control transcriptomic signature by a supervised classification algorithm.

Thus, in some embodiments, methods in accordance with the invention include:
i. providing a fecal RNA sample,
ii. determining the levels in the sample of a plurality of human gene products, thereby obtaining the transcriptomic signature of the sample with respect to the plurality of gene products and
iii. comparing the transcriptomic signature of said sample to a control transcriptomic signature by a supervised classification algorithm.

In one aspect, there is provided a method of analyzing a fecal RNA sample, comprising:
i. providing a fecal RNA sample, by a method comprising:
   a) providing a frozen stool sample that had been stored at a temperature not higher than −20° C. within 1 hour of sample collection (defecation),
   b) recovering RNA from said sample to provide a fecal RNA sample, and
   c) subjecting said fecal RNA sample to selective depletion of microbial rRNA, and
ii. determining the level of at least one human gene product in the fecal RNA sample.

In one embodiment, step b) is performed within 24 hours of step a). In another embodiment said stool sample had been stored at a temperature of about −80° C. within 12 hours of sample collection. In another embodiment said stool sample had been maintained (stored) at a temperature of about −80° C. for a period of between 6 hours and one month (prior to step b)). In another embodiment selective microbial rRNA depletion is performed by RNase H-based RNA depletion. In another embodiment selective microbial rRNA depletion comprises RNase H-based RNA depletion of 5S, 16S and 23S rRNA of gram-positive and gram-negative bacteria. In another embodiment, step c) is performed within 24 hours of step a). In another embodiment said stool sample had been maintained at a temperature of about −80° C. for a period of between 6 hours and one month. In another embodiment the stool sample had been obtained from a subject afflicted with, or suspected of having, gastrointestinal (GI) inflammation. In another embodiment the subject is diagnosed with, or suspected of having, an inflammatory bowel disease (IBD). In another embodiment said at least one human gene product is selected from Table 1 or from Table 4. In another embodiment step (ii) comprises determining the levels in the sample of a plurality of the human gene products, thereby obtaining the transcriptomic signature of the sample with respect to the plurality of gene products. In another embodiment, the method further comprises comparing the transcriptomic signature of said sample to a control transcriptomic signature by a supervised classification algorithm. In another embodiment the plurality of gene products is selected from the Table 4. In another embodiment the plurality of gene products is selected from Table 1. In another embodiment said plurality of gene products is as disclosed herein. In another embodiment the stool sample is stored at a temperature not higher than −20° C. within 1 hour of sample collection in the presence of an RNA preservation reagent comprising ammonium sulfate and EDTA and in the absence of a cryoprotectant.

In another embodiment, there is provided a method of analyzing a fecal RNA sample, comprising:
i. providing a fecal RNA sample, by a method comprising:
   a) providing a stool sample of a subject afflicted with, or suspected of having, GI inflammation,
   b) storing the stool sample at a temperature not higher than −20° C. within 1 hour of sample collection,
   c) recovering RNA from said sample to provide a fecal RNA sample, by thawing the frozen sample and isolating RNA from the thawed sample,
   d) subjecting said RNA sample to RNase H-based selective depletion of 5S, 16S and 23S rRNA of gram-positive and gram-negative bacteria,
   e) performing reverse transcription of the resulting depleted RNA, and
   f) generating a library of gene products using RNA barcoding, and sequencing (the generated library), and
ii. determining the level of at least one human gene product in the fecal RNA sample.

In another embodiment, step c) is performed within 24 hours of step a). In another embodiment said stool sample had been maintained at a temperature of about −80° C. for a period of between 6 hours and one month. In another embodiment the stool sample had been obtained from a subject afflicted with, or suspected of having, gastrointestinal (GI) inflammation. In another embodiment the subject is diagnosed with, or suspected of having, an inflammatory bowel disease (IBD). In another embodiment, step (ii) comprises determining the levels in the sample of a plurality of the human gene products, thereby obtaining the transcriptomic signature of the sample with respect to the plurality of gene products. In another embodiment the method further comprises comparing the transcriptomic signature of said sample to a control transcriptomic signature by a supervised classification algorithm. In another embodiment the plurality of gene products is selected from the Table 4. In another embodiment the plurality of gene products is selected from Table 1. In another embodiment said plurality of gene products is as disclosed herein.

In another embodiment the plurality of human gene products is selected from the group consisting of:
AC007192.1, ACSL1, ADGRG3, ALDOB, ALOX5AP, ALPL, AMN, ANP32E, AOC1, APBB1IP, APOA1, APOA4, APOB, APOC3, AQP9, ARHGAP26, ARHGAP30, ARRB2, BCL6, BID, CAPZA1, CASP5, CCL3, CCL3L3, CCL4, CCL4L2, CCR1, CD44, CD53, CD83, CDHR2, CDHR5, CLEC2B, CLEC4E, CLEC7A, CMTM2, CREB3L3, CREM, CSF3R, CXCL8, CXCR2, CYP3A4, CYTH1, CYTH4, DDX21, DEFA1B, DEFA3, DEFA5, DEFA6, DNAJA1, DPEP1, EIF3J, EPS8L1, ERICH1, EWSR1, FABP2, FABP6, FAM129A, FCER1G, FCGR2A, FCGR3A, FCGR3B, FNBP1, FPR1, FYB1, GOS2, GBP1, GBP4, GBP5, GDF5OS, GMFG, GNA11, GPR65; GUCA2A, GUCA2B, HCAR2, HCAR3, HNRNPA2B1, ICAM1, IFI16, IFIT2, IFIT3, IFITM2, IGSF6, IL1B, IL1R2, IL1RAP, IL1RN, ILF3, ITGAX, ITSN2, KCNJ15, KCNK6, KIAA0825, KIAA1109, LCP1, LCP2, LILRB3, LRRK2, LSP1, LYN, MAVS, MEP1A, METAP2, MNDA, MTRNR2L1, MTTP, NAMPT, NBN, NCF2, NCL, OLR1, OSM, PDE4B, PFKFB3, PHACTR1, PHIP, PLAU, PLCB2, PLCD3, PLEK, PPIF, PROK2, PTGS2, RAPGEF6, RCC1, REG1A, REG1B, RHOH, S100A4, S100A9, SAMSN1, SELENOP, SELL, SH3BP5, SI, SIPA1L2, SLC15A1, SLC2A3, SLC5A1, SMIM24, SNX10, SOCS3, SOD2, SP140, SRGN, SUPT6H, SYNE2, TANK, TET3, TLR2, TM4SF5, TMEM154, TNFAIP6, TRA2B, TREM1, UTRN, VNN2, WDR66, YTHDC1, ZEB2, ZFC3H1, ZNF267, and ZNF511-PRAP1.

In another embodiment said plurality of human gene products comprises:
a. TET3, RAPGEF6, RCC1, IL1R2, WDR66, ANP32E, EPS8L1, ALPL, ILF3, ERICH1, PLCD3, MAVS, SIPA1L2, ARHGAP30, FNBP1, NCL, EWSR1, SP140, DDX21, KCNK6, KIAA0825, SUPT6H, CYTH1, EIF3J, ARHGAP26, MTRNR2L1, PLCB2, UTRN, METAP2, and GDF5OS (Group A gene products);
b. REG1A, FABP6, REG1B, APOB, ALDOB, ZNF511-PRAP1, SI, CYP3A4, APOA1, DEFA6, APOA4, DPEP1, DEFA5, APOC3, CREB3L3, AMN, SLC15A1, GUCA2A, SMIM24, MTTP, SLC5A1, FABP2, MEP1A, TM4SF5, GUCA2B, AOC1, CDHR5, CDHR2, SELENOP, and GNA11 (Group B gene products);
c. AC007192.1, ACSL1, ALOX5AP, AQP9, BCL6, CCL4, CD44, CSF3R, CXCL8, DEFA1B, FAM129A, FCGR2A, FCGR3B, FPR1, FYB1, GOS2, GBP1, GMFG, HCAR2, HCAR3, ICAM1, IFI16, IFITM2, IL1B, IL1RN, ITGAX, LCP1, LCP2, LILRB3, LYN, MNDA, NAMPT, NCF2, OSM, PDE4B, PFKFB3, PLEK, PPIF, PROK2, PTGS2, S100A4, S100A9, SAMSN1, SLC2A3, SOCS3, SOD2, SRGN, TNFAIP6, TREM1, and ZNF267 (Group C gene products);
d. CAPZA1, CASP5, DNAJA1, HNRNPA2B1, ITSN2, KIAA1109, PHIP, SYNE2, TANK, TRA2B, YTHDC1, and ZFC3H1 (Group D gene products);
e. GBP5, DEFA3, DEFA1B, FCGR3A, CLEC2B, SH3BP5, CD53, CLEC7A, PLAU, IL1RAP, CCR1, FCER1G, CLEC4E, TLR2, LSP1, ADGRG3, CD83, TREM1, CMTM2, RHOH, APBB1IP, LRRK2, BID, KCNJ15, IFIT2, PLEK, PDE4B, HCAR3, ITGAX, SELL, MNDA, HCAR2, ICAM1, FCGR2A, CCL4L2, CCL3L3, GBP1, CSF3R, CD44, PROK2, SOCS3, GMFG, S100A4, TNFAIP6, SNX10, NBN, OSM, SOD2, IFI16, FYB1, AC007192.1, FCGR3B, IL1B, CYTH4, IL1RN, OLR1, VNN2, CCL3, CCL4, IFIT3, CREM, ZEB2, ALOX5AP, CXCL8, LCP2, IGSF6, CXCR2, ZNF267, GBP4, LCP1, PHACTR1, ARRB2, TMEM154, BCL6, and GPR65 (Group E gene products); and/or
f. GBP5, DEFA3, DEFA1B, FCGR3A, CLEC2B, SH3BP5, CD53, CLEC7A, PLAU, IL1RAP, CCR1, FCER1G, CLEC4E, TLR2, LSP1, ADGRG3, CD83, TREM1, CMTM2, RHOH, APBB1IP, LRRK2, BID, KCNJ15, IFIT2, PLEK, PDE4B, HCAR3, ITGAX, SELL, MNDA, HCAR2, ICAM1, FCGR2A, and CCL4L2 (Group F gene products).

In another embodiment, said plurality of human gene products comprises:
e. RNASEK, RNASEK-C17orf49, MIDN, HLA-A, B2M, HLA-B, HLA-C, CAP1, PFN1, ACTB, MXD1, SAT1, LITAF, NFKBIA, S100A9, S100A8, EIF1, FTL, and FTH1 (Group G gene products),
f. AC138811.2, AQP9, ARPC2, ARPC5, BASP1, BCL2A1, BRI3, BTG2, CALM2, CCL4, CDKN1A, CEACAM1, CEBPB, CXCL8, EGR1, ETS2, FOS, FPR1, FTL, GOS2, GABARAP, GLUL, GNB2, HCAR3, HLA-C, HLA-E, ICAM1, IFITM1, IFITM2, IL1B, IL1RN, IRF1, ISG20, ITM2B, IVNS1ABP, KDM6B, KLF6, LITAF, MARCKS, MCL1, MXD1, NAMPT, NFKBIA, OSM, PFN1, PLAUR, PLEK, PNRC1, PPIF, PROK2, PTP4A1, S100A11, S100A8, S100A9, SAT1, SDCBP, SLC25A37, SOCS3, SOD2, SRGN, TAF10, TNFAIP3, TPM4, TXNIP, TYMP, UBE2B, VASP, WDR83OS, ZFP36, and ZFP36L1 (Group H gene products),
g. CCL4, CXCL8, HCAR3, ICAM1, IL1B, IL1RN, OSM, PLEK, PROK2, SOCS3, SOD2 (Group I gene products), and/or
h. ABHD17C, AC005943.1, ACTN4, ALDOA, AP000350.4, AP000721.1, AP003419.1, C17orf49, CA4, CDHR5, CFL1, CKB, COX7A2, COX8A, CRIP1, CST3, CTNND1, DBNDD2, DYNLRB1, EGLN1, EIF4G2, EPCAM, FABP1, FCGBP, FXYD3, GUCA2A, GUCA2B, IFI27, KRT8, LGALS4, LYPD8, MAL2, MGLL, MIF, MISP, MUC12, MUC2, MYL12B, OST4, P2RX5-TAX1BP3, PDLIM1, PHGR1, PIGR, PLAC8, POLD4, PPDPF, RHOC, S100A16, SDCBP2, SERINC2, SFN, SH3BGRL3, SMIM22, SPINT2, SRI, STK24, SYS1-DBNDD2, TAX1BP3, TFF1, TFF3, TMEM54, TMPRSS2, TMSB10, TRIM31, UBA52, UBB, UQCR11, ZG16 (Group J gene products).

In another embodiment, the outcome of the comparison is indicative of the location of GI inflammation in said subject. In another embodiment said plurality of human gene products is as set forth in Group A and/or B.

In another embodiment, the outcome of the comparison is indicative of the severity of GI inflammation in said subject. In another embodiment said plurality of human gene products is as set forth in Group C and/or D.

In another embodiment the outcome of the comparison is indicative of the presence or absence of GI inflammation in said subject. In another embodiment said plurality of human gene products is as set forth in Group E, F, G, H, I and/or J. In another embodiment said plurality of human gene products is as set forth in Group E or F.

In another embodiment, the stool sample is stored at a temperature not higher than −20° C. within 1 hour of sample collection in the presence of an RNA preservation reagent comprising ammonium sulfate and EDTA and in the absence of a cryoprotectant.

In another aspect, the invention provides a method of analyzing a fecal RNA sample (e.g. for the presence, location and/or severity of GI inflammation), comprising:
i. providing a fecal RNA sample of a subject afflicted with, or suspected of having, GI inflammation,
ii. determining the levels in the sample of a plurality of human gene products, thereby obtaining the transcriptomic signature of the sample with respect to the plurality of gene products, and
iii. comparing the transcriptomic signature of said sample to a control transcriptomic signature by a supervised classification algorithm,
wherein said plurality of gene products is selected from the group consisting of: Group A gene products, Group B gene products, Group C gene products, Group D gene products, Group E gene products, Group F gene products, Group G gene products, Group H gene products, Group I gene products, and/or Group J gene products. In another embodiment the plurality of gene products further comprises at least one additional gene product selected from Table 4.

In another aspect, there is provided a method of determining the location of gastrointestinal (GI) inflammation, comprising:
i. providing a fecal RNA sample of a subject afflicted with, or suspected of having, GI inflammation,
ii. determining the levels in the sample of a plurality of human gene products, thereby obtaining the transcriptomic signature of the sample with respect to the plurality of gene products, and
iii. comparing the transcriptomic signature of said sample to a control transcriptomic signature by a supervised classification algorithm,
   wherein the outcome of the comparison is indicative of the location of GI inflammation in said subject.

In another embodiment the plurality of gene products is selected from the Table 4. In another embodiment said plurality of gene products is as disclosed herein. In another embodiment, said plurality of gene products is selected from the group consisting of: a first plurality of gene products comprising Group A gene products, and/or a second plurality of gene products comprising: Group B gene products. In another embodiment, said subject is suspected of having colonic GI inflammation or terminal ileal GI inflammation. In another embodiment said subject is suspected of having colonic Crohn's disease (CD) or terminal ileal CD. In another embodiment step (ii) comprises determining the transcriptomic signatures of the sample with respect to the first plurality of gene products of Group A and the second plurality of gene products of Group B. In another embodiment the control transcriptomic signature comprises at least one of: a control transcriptomic signature with respect to the plurality of gene products corresponding to colonic CD that does not involve the terminal ileum, and a control transcriptomic signature with respect to the plurality of gene products corresponding to terminal ileal CD.

In another embodiment the method comprises comparing said transcriptomic signatures to a first control transcriptomic signature corresponding to colonic CD that does not involve the terminal ileum and to a second control transcriptomic signature corresponding to terminal ileal CD. In another embodiment said transcriptomic signature reflects the collective levels of said first plurality of gene products of Group A in said sample as compared to the collective levels of said second plurality of gene products of Group B in said sample, wherein
the higher the enhancement of the collective levels of said first plurality of gene products in said sample as compared to the collective levels of said second plurality of gene products in said sample, the higher the probability that said subject is afflicted with colonic CD that does not involve the terminal ileum, and/or
the higher the enhancement of the collective levels of said second plurality of gene products in said sample as compared to the collective levels of first plurality of gene products in said sample, the higher the probability that said subject is afflicted with terminal ileal CD.

In another embodiment, a transcriptomic signature characterized by significant enhancement of the collective levels of said first plurality of gene products in said sample as compared to a control transcriptomic signature corresponding to terminal ileal CD indicates that said subject is afflicted with colonic CD, and/or a transcriptomic signature characterized by significant enhancement of the collective levels of said second plurality of gene products in said sample as compared to a control transcriptomic signature corresponding to colonic CD that does not involve the terminal ileum indicates that said subject is afflicted with terminal ileal CD.

In another aspect, there is provided a method of determining the severity of GI inflammation, comprising:
i. providing a fecal RNA sample of a subject afflicted with, or suspected of having, GI inflammation,
ii. determining the levels in the sample of a plurality of human gene products, thereby obtaining the transcriptomic signature of the sample with respect to the plurality of gene products, and
iii. comparing the transcriptomic signature of said sample to a control transcriptomic signature by a supervised classification algorithm,
wherein said plurality of gene products is selected from the group consisting of: a first plurality of gene products comprising Group C gene products and/or a second plurality of gene products comprising Group D gene products, and wherein the outcome of the comparison is indicative of the severity of GI inflammation in said subject.

In another embodiment step (ii) comprises determining the transcriptomic signature of the sample with respect to the first plurality of gene products of Group C and the second plurality of gene products of Group D. In another embodiment the control transcriptomic signature comprises control transcriptomic signature with respect to the plurality of gene products corresponding to at least one of: a healthy control, inactive GI inflammation, mild GI inflammation, moderate GI inflammation, and severe GI inflammation. In another embodiment the control transcriptomic signature comprises control transcriptomic signature with respect to the plurality of gene products corresponding to at least one of: a healthy control, GI inflammation characterized by an endoscopic score of at least 2, and GI inflammation characterized by an endoscopic severity lower than 2, wherein said endoscopic severity is determined according to the Mayo and/or Simple Endoscopic Score (SESCD) scores.

In another embodiment said transcriptomic signature reflects the collective levels of said first plurality of gene products of Group C in said sample as compared to the collective levels of said second plurality of gene products of Group D in said sample, and wherein the higher the enhancement of said collective levels of said first plurality of gene products in said sample as compared to said collective levels of said second plurality of gene products in said sample, the higher the predicted severity of GI inflammation in said subject.

In another aspect, the invention provides a method of identifying GI inflammation, comprising:
i. providing a fecal RNA sample of a subject suspected of having GI inflammation,
ii. determining the levels in the sample of a plurality of human gene products, thereby obtaining the transcriptomic signature of the sample with respect to the plurality of gene products, and
iii. comparing the transcriptomic signature of said sample to a control transcriptomic signature by a supervised classification algorithm,
   wherein said plurality of gene products is selected from the group consisting of Group E, F, G, H and/or I gene products, and wherein the outcome of the comparison is indicative of the presence or absence of GI inflammation in said subject.

In another embodiment said plurality of gene products comprises Group E or Group F gene products. In another embodiment the control transcriptomic signature corresponds to a healthy control, and a transcriptomic signature characterized by significant enhancement of the collective levels of said plurality of gene products in said sample (e.g. of Group E, F, G, H and/or I) as compared to said healthy control indicates that said subject is afflicted with GI inflammation.

In another embodiment said plurality of gene products further comprises Group J gene products. In another embodiment the control transcriptomic signature corresponds to a healthy control, and a transcriptomic signature characterized by significant enhancement of the collective levels of gene products selected from Group E, F, G, H and/or I, and by a significant reduction of the collective levels of gene products selected from Group J, in said sample as compared to said healthy control, indicates that said subject is afflicted with GI inflammation.

In another embodiment of the diagnostic methods of the invention, said sample is a fecal wash sample. In a particular embodiment said sample is a distal fecal wash sample. In another embodiment of the diagnostic methods of the invention, said sample is a stool sample. In another embodiment, providing said fecal RNA sample comprises:
a) providing a frozen stool sample that had been stored at a temperature not higher than −20° C. within 1 hour of sample collection,
b) recovering RNA from said sample to provide a fecal RNA sample, and
c) subjecting said fecal RNA sample to selective depletion of microbial rRNA.

In another embodiment providing said fecal RNA sample comprises:
a) providing a stool sample of a subject afflicted with, or suspected of having, GI inflammation,
b) storing the stool sample at a temperature not higher than −20° C. within 1 hour of sample collection (and optionally at a temperature of about −80° C. no later than 12 hours of sample collection),
c) recovering RNA from said sample to provide a fecal RNA sample, by thawing the frozen sample and isolating RNA from the thawed sample,
d) subjecting said RNA sample to RNase H-based selective depletion of 5S, 16S and 23S rRNA of gram-positive and gram-negative bacteria,
e) performing reverse transcription of the resulting depleted RNA, and
f) generating a library of gene products using RNA barcoding, and sequencing the generated library.

In another embodiment of the methods of the invention, the subject is afflicted with, or suspected of having, ulcerative colitis (UC). In another embodiment the subject is afflicted with, or suspected of having, Crohn's disease (CD). In another embodiment of the methods of the invention, determining the levels of each gene product in the sample comprises determining the Unique Molecular Identifier (UMI) counts for each gene product. In another embodiment determining the levels of each gene product in the sample further comprises normalizing the level of each UMI in said sample to the levels of other UMIs in said sample. In another embodiment the control transcriptomic signature corresponds to at least one control individual, a panel of control samples from a set of control individuals, or a stored set of data of control individuals.

In another aspect the invention provides a method of determining one or more human gene products from a stool sample, comprising:
a) providing a frozen stool sample that has been stored at a temperature not higher than −20° C. within 1 hour of sample collection,
b) recovering RNA from said sample to provide a fecal RNA sample,
c) selectively depleting microbial rRNA from the fecal RNA sample, and
d) analyzing the depleted fecal RNA sample of step c) to determine one or more human gene products therein,
wherein step (d) comprises determining the levels in the sample of a plurality of the human gene products selected from Table 1 or from Table 4, thereby obtaining the transcriptomic signature of the sample with respect to the plurality of gene products.

In another aspect, the invention provides a diagnostic kit, comprising means for specifically determining and quantifying the levels of a plurality of human gene products in a fecal RNA sample, wherein the plurality of human gene products comprises or consists of: Group A gene products, Group B gene products, Group C gene products, Group D gene products, Group E gene products, Group F gene products, Group G gene products, Group H gene products, Group I gene products, and/or Group J gene products.

In another embodiment, said plurality of gene products comprises or consists of Group A, B, C, D, E and/or F gene products. In another embodiment the means comprise quantitative polymerase chain reaction (qPCR) primers directed to said plurality of human gene products. In another embodiment the kit further comprising means for providing a fecal RNA sample and/or means for comparing the levels of said plurality of human gene products in the sample to their levels in a control fecal RNA sample.

In another embodiment said plurality of human gene products is as set forth in Group A and/or Group B, and the kit is used for determining the location of GI inflammation in a subject and/or comprises instructions for use in determining the location of GI inflammation in a subject.

In another embodiment said plurality of human gene products is as set forth in Group C and/or Group D, and the kit is used for determining the severity of GI inflammation in a subject and/or comprises instructions for use in determining the severity of GI inflammation in a subject.

In another embodiment said plurality of human gene products is as set forth in Group E, F, G, H, I and/or J, and the kit is used for determining the presence or absence of GI inflammation in a subject and/or comprises instructions for use in determining the presence or absence of GI inflammation in a subject. In a particular embodiment said plurality of human gene products is as set forth in Group E or in Group F.

### Fecal Samples

In one embodiment, the subject is afflicted with, or suspected of having, GI inflammation. In another embodiment, the subject is afflicted with IBD. In another embodiment, the subject is diagnosed with IBD. In another embodiment said subject is suspected of having IBD (e.g. CD or UC). In some embodiments, the sample is a fecal wash sample (e.g. a distal (left-sided) fecal wash). In other embodiments, the sample is a solid fecal sample (stool sample).

In some embodiments, the methods of the invention comprise improved means for providing fecal RNA samples from stool samples. In some embodiments, the stool sample is provided in the form of a frozen stool sample that had been stored at a temperature not higher than about −20° C. within 1 hour of sample collection (namely within 1 hour of excretion of said sample). For example, the sample may be stored a temperature not higher than −20° C. within 10-60 minutes, e.g. within 10, 20, 30, 40, 50 or 60 minutes of collection. In some embodiments, said sample has been stored at a temperature of about −20° C. to about −80° C., e.g. −20° C. to −40° C., −20° C. to −30° C., −70° C. to −80° C., or about −20° C., −40° C., −70° C. or −80° C., within one hour of collection (defecation). In another embodiment, the sample is subjected to cryopreservation within one hour of collection. In another embodiment, said sample is maintained at about −20° C. for up to 24 hours of collection until further processing (e.g. RNA isolation). In another embodiment, said sample is maintained at about −80° C. In another embodiment, the method further comprises recovering RNA from said sample (e.g. within 24 hours of collection if maintained at about −20° C.) to provide a fecal RNA sample. Typically, recovering RNA from the frozen sample comprises thawing the sample and isolating RNA from the thawed sample.

The term “fecal wash” refers to fecal material which is removed from the body in a liquid state. In one embodiment, the fecal fluid is suctioned from the subject during colonoscopy or sigmoidoscopy or gastroscopy, including fluid suctioned from the small or large intestine. Fecal wash can also be obtained via rectal tube suctioning, with or without rectal irrigation. In another embodiment, the fecal wash refers to a liquid stool sample collected by the patient after consumption of a laxative. In another embodiment, the sample is a distal (left-sided) fecal wash, suctioned from the distal colon during colonoscopy.

In another embodiment the sample is a solid fecal (stool) sample provided in a frozen state. The term “frozen sample” means a sample in a cryopreserved state. The frozen sample is preferably a sample which has been preserved at a temperature of up to −20° C. or −80° C. for a certain period of time such as several hours to several weeks or longer. In some embodiments, the frozen sample had been stored at a temperature not higher than −20° C. within an hour of collection and up to about 12 hours, 24 hours, 2 days, 3 days, 5 days, 6 days, 7 days, 10 days, 2 weeks, 3 weeks or 4 weeks.

The term “cryopreservation”, as used herein, refers to the process of cooling and storing biological samples at a temperature below the freezing point of water. In the context of the present invention, cryopreservation refers in particular to processes in which the frozen biological sample is stored at a temperature of −20° C. or colder (or in other embodiments −80° C. or colder, or −20° C. to −80° C.). Without wishing to be bound by a specific theory or mechanism of action, cryopreservation according to the methods and processes of the invention facilitates reduction, delay or prevention of deterioration of biomolecules such as RNA in the sample. In some embodiments, the cryopreservation process involves treating the biological sample with a reagent prior to freezing, and storing the resulting frozen sample (e.g. at a temperature not higher than −20° C. or −80° C.) in the presence of the reagent. In some embodiments, the cryopreservation reagent may be e.g. an RNA preservation reagent and/or a cryoprotectant.

As disclosed herein, cryopreservation reagents particularly suitable for use in embodiments of the invention include RNA preservation reagents that protect the RNA in the sample from nucleases (e.g. by nuclease inactivation). Exemplary RNA preservation reagents to be used in embodiments of the invention include solutions comprising at least one salt, e.g. a sulfate salt such as ammonium sulfate ((NH₄)₂SO₄), and having a total salt concentration of between 10 g/100 ml and the saturating concentration of the salt. Typically, ammonium sulfate is present in the reagent at a concentration range of 1.2-1.6 M. Without wishing to be bound by a specific theory or mechanism of action, the sample is exposed to high ammonium sulfate concentrations so as to facilitate precipitation of proteins (including RNases) and stabilization of RNA by creating a denaturing environment. In a particular embodiment the ammonium sulfate in a concentration of between 30 g/100 ml and 80 g/100 ml. In various embodiments, said RNA preservation reagent further comprises a chelator of divalent cations, a buffer and/or an organic solvent. In some embodiments, said RNA preservation reagent has a pH of between 4 and 8 (e.g. 5.0-7.5 or 6.8-7.0). For example, the RNA preservation reagent may contain ethylenediaminetetraacetic acid (EDTA) e.g. at 1-10 mM. Without wishing to be bound by a specific theory or mechanism of action, the sample may be exposed to EDTA or other suitable chelators so as to reduce the concentrations of divalent metal ions (Mg²⁺, Ca²⁺) required for RNase activity. In other embodiments, the reagent may comprise Tris-HCl buffer (e.g. at 10-50 mM) and optionally other salts such as sodium chloride or potassium chloride (Typically at concentrations of 50-150 mM if present), which may maintain ionic strength and stability.

In a particular embodiment, the reagent may contain 20-30 mM sodium citrate, 5-15 mM EDTA, 60-80 g ammonium sulfate per 100 ml solution, and has a pH of 5-6. In a particular embodiment, the reagent may contain 1.2-1.6 M ammonium sulfate, 1-10 mM EDTA, and 10-50 mM Tris-HCl buffer, and has a pH of 6.8-7.0. In some embodiments, the RNA preservation reagent may conveniently be added to a solid fecal sample at a volume of about 5-15 fold (e.g. 10-fold) greater than the volume of the fecal sample.

For example, reagents such as RNAlater may be used. In general, the term “RNAlater™” is employed to denote the formulation disclosed in Examples 3 and 5 herein, which is composed of 25 mM sodium citrate, 10 mM EDTA, 70 g ammonium sulfate/100 ml solution, pH 5.2. Additional reagents amenable for cryopreservation of samples are described e.g. in U.S. Pat. No. 6,204,375B1.

In other embodiments, cryoprotectants (that protect the sample and its associated compounds from freezing damage) may optionally be used. Such cryoprotectants are known in the art and include for example dimethyl sulfoxide (DMSO), glycerol, ethylene glycol and propanediol. However, it is to be understood that the use of cryoprotectants is not mandatory in the context of the processes of the invention, and in some embodiments cryopreservation of the sample may be conveniently be performed in the absence of cryoprotectants. In yet other embodiments, the processes of the invention may be performed so as to minimize the exposure of the sample to cryoprotectants upon thawing (for example, by using low DMSO concentrations and/or rapid processing of the thawed sample so as to remove the DMSO or other cryoprotectants). In a particular embodiment, the sample is subjected to cryopreservation in the presence of DMSO (e.g. at low concentrations of up to about 5%). In another embodiment the sample is subjected to cryopreservation in the absence of cryoprotectants (e.g. DMSO). Each possibility represents a separate embodiment of the invention.

In some embodiments, the stool sample is stored at a temperature not higher than-20° C. within 1 hour of sample collection in the presence of an RNA preservation reagent comprising ammonium sulfate and EDTA and in the absence of a cryoprotectant.

Thus, storing a stool sample at a temperature not higher than −20° C. within 1 hour of sample collection, typically includes cryopreservation of said sample in the presence of a RNA preservation reagents as disclosed herein, at said temperature within an hour of said sample being extracted from the body. The term “storing” further indicates that the cryopreserved sample is maintained at said temperature, e.g. using suitable freezing devices such as domestic freezers (for −20° C.) or ultra-low temperature freezer (for −80° C.).

The term “thawing” as used herein refers to raising the temperature of the cryopreserved composition or biological material in this case cells, to 0° C. or above, preferably to 4° C. or above. It is to be understood that in the presence of certain reagents such as some of the cryoprotectants and RNA preservation reagents disclosed herein, and depending on the duration and temperature of cryopreservation, a frozen sample does not necessarily reach a solid state prior to thawing.

### Fecal RNA Preparation

In some embodiments, step (i) comprises subjecting said fecal RNA sample to selective depletion of microbial rRNA. According to exemplary embodiments, selective depletion of microbial rRNA is performed by RNase H-based RNA depletion. In another embodiment the microbial rRNA comprises 5S, 16S and/or 23S rRNA from gram-positive and/or gram-negative organisms. In another embodiment selective depletion of microbial rRNA comprises RNase H-based RNA depletion of 5S, 16S and 23S rRNA of gram-positive and gram-negative bacteria. In another embodiment, step (i) further comprises reverse transcription of the RNA following the depletion step. In another embodiment step (i) further comprises gene sequencing. In another embodiment, step (i) further comprises barcoding said gene products. In another embodiment, step (i) further comprises generating a library of gene products using RNA barcoding, and sequencing (the generated library).

Thus, in some embodiments, providing the fecal RNA sample is performed by a method comprising:
a) providing a frozen stool sample that had been stored at a temperature not higher than −20° C. within 1 hour of sample collection,
b) recovering RNA from said sample to provide a fecal RNA sample, and
c) subjecting said fecal RNA sample to selective depletion of microbial rRNA.

In another embodiment there is provided a method of analyzing a fecal RNA sample, comprising:
i. providing a fecal RNA sample, by a method comprising:
   a) providing a frozen stool sample that had been stored at a temperature not higher than −20° C. within 1 hour of sample collection,
   b) recovering RNA from said sample to provide a fecal RNA sample, and
   c) subjecting said fecal RNA sample to selective depletion of microbial rRNA, and
ii. determining the level of at least one human gene product in the fecal RNA sample.

In another embodiment, step b) is performed within 24 hours of step a). In another embodiment, said stool sample had been stored at a temperature of about −80° C. within 12 hours of sample collection. In another embodiment, said stool sample had been maintained at a temperature of about-80° C. for a period of between 6 hours and one month.

In another embodiment, step (i) comprises:
a) providing a stool sample of a subject afflicted with, or suspected of having, GI inflammation,
b) storing the stool sample at a temperature not higher than −20° C. within 1 hour of sample collection and at a temperature of about-80° C. no later than 12 hours of sample collection,
c) recovering RNA from said sample to provide a fecal RNA sample, by thawing the frozen sample and isolating RNA from the thawed sample,
d) subjecting said RNA sample to RNase H-based selective depletion of 5S, 16S and 23S rRNA of gram-positive and gram-negative bacteria,
e) performing reverse transcription of the resulting depleted RNA, and
f) generating a library of gene products using RNA barcoding, and sequencing.

In another embodiment, step (i) comprises:
a) providing a stool sample of a subject afflicted with, or suspected of having, IBD,
b) subjecting the sample to cryopreservation within one hour of sample collection,
c) isolating RNA from said sample to provide a fecal RNA sample,
d) subjecting said RNA sample to RNase H-based selective depletion of 5S, 16S and 23S rRNA of gram-positive and gram-negative bacteria,
e) performing reverse transcription of the resulting RNA to obtain a depleted stool cDNA sample, and
f) generating a library of gene products using RNA barcoding, and sequencing.

As is used herein, the term “isolating RNA” refers to the extraction and purification of RNA from a biological sample. The term “isolating” further refers to the removal of other components, such as proteins and DNA, at least to some extent. Various means for RNA isolation are available, as described below.

As disclosed herein, fecal RNA samples of the invention are typically subjected to specific or preferential removal of ribosomal RNA corresponding to microorganisms present in the sample (such that other types of RNA in the sample such as mRNA are retained), also referred to herein as “selective depletion of microbial rRNA” or “negative genomic selection of microbial rRNA”.

In particular, fecal RNA samples obtained from solid fecal samples (stool samples) are subjected to selective depletion of microbial rRNA. Thus, the percentage of a type of rRNA (or one or more particular sub-types thereof) in the sample is reduced with respect to the total nucleic acid in said sample. In the context of the invention, the fraction of human exonic reads in the sequenced samples may be increased. In particular embodiments, the depleted sample contains no more than 20% microbial rRNA and typically less than 10%, 9%, 8%, 7%, 6%, 5%, 4%, 3%, 2%, or 1%, including 0.5%, 0.1%, 0.01% or less. In another embodiment, the microbial rRNA is not detectable in the treated sample by conventional methods such as PCR.

In particular, embodiment selective microbial rRNA depletion may be performed by RNase H-based RNA depletion. In other particular embodiments, said depletion may include selective depletion of 5S, 16S and/or 23S rRNA. In another particular embodiment, selective microbial rRNA depletion comprises RNase H-based RNA depletion of 5S, 16S and 23S rRNA of gram-positive and gram-negative bacteria.

Bacterial ribosomes contain three distinct RNA molecules referred to as 5S, 16S and 23S rRNAs. These names historically are related to the size of the RNA molecules, as determined by their sedimentation rate (e.g. in E. coli). While ribosomal RNA molecules vary substantially in size between organisms, 5S, 16S, and 23S rRNA are commonly used as generic names for the homologous RNA molecules in any bacterium, and this convention is referred to herein. These homologous regions may conveniently be targeted (e.g. by suitable nucleic acid probes) for selective depletion.

Advantageously, selective rRNA depletion includes depletion of 5S, 16S, and 23S rRNAs from both Gram-positive bacteria (characterized by a thick peptidoglycan layer and no outer lipid membrane detectably by Gram staining) and Gram-negative bacteria (having a thin peptidoglycan layer and have an outer lipid membrane). The 5S, 16S, and 23S rRNA of Gram positive and Gram-negative bacteria are readily available to the skilled artisan.

Conveniently and advantageously, selective depletion of microbial rRNA is effected by methods employing the use of ribonuclease H (RNAse H) digestion, which are referred to herein as “RNase H-based selective rRNA depletion”. For example, DNA probes may be synthesized to be reverse-complement to the bacterial or fungal transcripts. Next, RNase H enzyme may be used which digests RNA-DNA specific hybrids. This leads to the selective digestion of only RNA molecules targeted by the DNA probes. Lastly, endonucleases such as DNase I enzyme may be used to remove the left over DNA probes and other DNA residues left in the sample after RNA extraction.

In some embodiments, the RNase H is RNase H1. In some embodiments, the RNAse H is a thermostable RNAse H. Thermostable RNAse H may be obtained commercially, including, for example, Hybridase™ (Lucigen, Middleton, Wis.).

In some embodiments, the RNAse H degrades the RNA from an RNA: DNA hybrid at a temperature range of between 32° C. and 95° C. (e.g., using a thermostable RNAse H). In some embodiments, the RNAse H degrades the RNA from an RNA: DNA hybrid at a temperature range of between 32° C. and 60° C. In some embodiments, the RNAse H degrades the RNA from an RNA: DNA hybrid at a temperature range of between 37° C. and 60° C. In another embodiment, the RNAse H degrades the RNA from an RNA: DNA hybrid at a temperature of range of between 36° C. and 38° C., e.g. 37° C.

In some embodiments, DNA probes that have not hybridized with target undesirable RNA, or probes that have been released following RNase H degradation of the RNA from the RNA: DNA hybrid, can be removed at various stages of RNA isolation by DNA degrading enzymes or other techniques well known in the art. In some embodiments, the DNA degrading enzyme is an exonuclease that digests DNA from in a 5′ to 3′ direction. In some embodiments, the DNA degrading enzyme does not digest the capture probes attached on the substrate.

Another method for depleting particular RNAs is by using nucleic acid probes (which are attached to an affinity tag) that specifically hybridize to the RNAs. Exemplary affinity tags include, but are not limited to hemagglutinin (HA), AviTag™, V5, Myc, T7, FLAG, HSV, VSV-G, His, biotin, or streptavidin. In some embodiments, commercially available reagents such as New England Biolabs NEBNext rRNA Depletion Kit or RNA depletion methods as described in U.S. Pat. Nos. 9,745,570 and 9,005,891 may be used.

In some embodiments, negative genomic selection of abundant microbial transcripts such as bacterial and/or fungal rRNA may be performed prior to the analysis. Examples of particular additional RNA transcripts that may be depleted include, but are not limited to Eubacterium rectale, Faecalibacterium prausnitzii, Bifidobacterium adolescentis, Ruminococcus sp 5 1 39BFAA, Bifidobacterium longum, Subdoligranulum, Ruminococcus gnavus, Escherichia coli, Ruminococcus torques, Akkermansia muciniphila, Ruminococcus bromii, Dialister invisus, Collinsella aerofaciens, Bacteroides uniformis, Bacteroides vulgatus, Eubacterium hallii, Dorea longicatena, Prevotella copri, Alistipes putredinis and Bifidobacterium bifidum. In some embodiments, rRNA of at least one, at least two, at least three, at least four or at least 5 or all of the above identified bacteria are depleted.

As used herein, a “depleted RNA” refers to an RNA sample that had been subjected to selective depletion of microbial rRNA.

The term “reverse transcription” is used according to its conventional meaning to denote the generation of a complementary DNA (cDNA) from an RNA template by an RNA-dependent DNA polymerase, in particular a reverse transcriptase (RT) enzyme.

The term “library of gene products” refers to a collection of molecules such as complementary DNA (cDNA) molecules or fragments thereof, which together constitute at least a portion of the transcriptome of a single cell or a plurality of single cells. More typically, libraries of gene products to be used cDNA may be produced by reverse transcription from fully transcribed mRNA found in a cell (and therefore contains only the expressed genes of a single cell), or, when pooled together, the expressed genes from a plurality of single cells. In some embodiments, generating the library further comprises assigning a unique molecular tag to each gene product in the library, by barcoding processes as described herein.

The term “barcode” or “barcoding” when used as a verb with reference to a reaction, indicates a reaction performed to covalently attach a barcode in the sense of the disclosure to the reference item, in a configuration allowing detection of the barcode. Accordingly, barcoding in the sense of the disclosure refers to coupling a unique set of tags or identifiers in order to mark molecules for downstream detection and identification. As used herein, “unique” means different from any other. The term “barcode” as used herein refers in particular to a short sequence of nucleotides (for example, DNA or RNA) that is used as an identifier for an associated molecule, such as a target molecule and/or target nucleic acid, or as an identifier of the source of an associated molecule, such as a cell-of-origin. Barcodes can allow for identification and/or quantification of individual sequencing-reads. In some embodiments, a barcode can be obtained by sequential direct covalent linkage of a tag with another tag until formation of a barcode comprising a series of two or more tags directly attached one to another through covalent linkage. In various embodiments, barcodes may be added to an mRNA molecule (e.g. during reverse transcription) and/or to a cDNA molecule (e.g. by appropriate protocols such as Truseq).

As is used herein, the term “RNA-Seq” also termed RNA-sequencing, refers to a sequencing technique, such as a high-throughput sequencing technique, typically using next-generation sequencing (NGS), to characterize the quantity and/or sequence of a nucleic acid molecule such as RNA in a sample. RNA-Seq can be used for gene expression analysis in accordance with the invention.

In some embodiments, the library of gene products is generated by a process compatible with RNA barcoding and sequencing protocols. In particular embodiments, 3′ mRNA-seq techniques may be used, in which short reads are generated only for the 3′ region of polyadenylated mRNA molecules instead of the full length of transcripts like in standard RNA-seq. In other particular embodiments, RNA barcoding and sequencing protocols utilizing polyethylene glycol (PEG) for enhancing the reverse transcription efficiency.

For example, without limitation, embodiments of the invention as demonstrated herein employ a modified single cell RNA barcoding and sequencing (SCRB-seq) protocol, adapted for use on cell-free RNA samples as disclosed and exemplified herein. SCRB-seq is an ultra-high-throughput bulk 3′ mRNA-seq technology that uses early-stage sample barcoding and unique molecular identifiers (UMIs) to allow the pooling of up to 8 samples in one tube early in the sequencing library preparation workflow.

In some embodiments, multiple barcodes may be added to each member of the gene product library. For example, in a gene product library composed of pooled single cells as disclosed herein, the RNA barcoding and sequencing may advantageously include attachment of both a molecule-specific UMI to mark each gene product, a sample-specific barcode to mark the source of the gene product, as described and exemplified below, and optionally a pool-specific (indexing) barcode marking the assigned group of gene products. Non-limitative examples of methods and protocols employing the assignment of multiple distinct barcodes providing improved diagnostic outcomes are specified and demonstrated herein. For instance, Example 3 below demonstrates the use of gene product-specific UMIs (10-mer random oligonucleotides), sample-specific barcodes (6-mer random oligonucleotides) and pool-specific barcodes (unique dual index (UDI) barcodes, for example Illumina UDIs). More specifically, barcode-containing primers that may be used in RNA barcoding protocols for constructing a library in accordance with the invention are exemplified by SEQ ID NOs: 2-33 (reverse transcription (RT) primers for introducing UMIs and sample-specific barcodes) and 35-82 (for introducing pool-specific index UDIs). In some embodiments, RNA barcoding methods according to the invention include incorporation of UMIs, sample-specific 6-mer barcodes, and pool-specific UDIs as described in Example 3. In some embodiments, the use of improved barcoded RT primers comprising UMIs and sample-specific 6-mer barcodes as set forth in Formula 1 is contemplated. According to particular embodiments, performing reverse transcription of the resulting depleted RNA and generating a library of gene products using RNA barcoding employ the use of primers corresponding to SEQ ID NOs: 2-33 and 35-82, and optionally SEQ ID NO: 1 (RT primer) and 34 (biotinylated primer).

As used herein, the term “fecal RNA sample” refers to a sample comprising or corresponding to at least a portion of the RNA transcriptome obtained from a fecal sample. A fecal RNA sample to be used in accordance with the invention is typically obtainable by a process comprising recovering RNA from a frozen stool sample (that had been advantageously stored at a temperature of −20° C. or lower within 1 hour of sample collection, typically by thawing the frozen sample and isolating RNA from the thawed sample) and subjecting said fecal RNA sample to selective depletion of microbial rRNA as disclosed herein. In some embodiments, the fecal RNA sample is further processed prior to analysis, for example by performing reverse transcription of the resulting depleted RNA, and generating a library of gene products corresponding to said resulting depleted RNA.

### Gene Expression Evaluation

In another embodiment step (ii) comprises determining the levels in the resulting sample (fecal RNA sample, or library of gene products corresponding thereto) of a plurality of the human gene products, thereby obtaining the transcriptomic signature of the sample with respect to the plurality of gene products.

As used herein, the term “plurality” relates to a group of three or more. The invention in embodiment thereof provides a number of groups of gene products (also referred to as a “plurality of gene products”), including for example those identified in Tables [2-3 herein, that are particularly useful in assessing various aspects of GI inflammation as disclosed herein, when used in the context of their group. For instance, several groups of gene products selected from Tables 1 and 4 were used for constructing the diagnostic classifiers as described in Examples [1, 2, 4 and 5. It is to be understood, that diagnostic classifiers may include a plurality of gene products comprising additional gene products (from Tables 1 and/or 4) or a subset of the gene products identified in Tables 2 and 3, to provide an accurate diagnosis to the particular case examined.

In various embodiments, the plurality of gene products to be used in methods and assays of the invention comprises or consists of at least 12 and typically at least 20 of said gene products, more typically at least 35 of said gene products, e.g. 35-75, 35-55, or 50-75 of said gene products. In other embodiments, said plurality of gene products comprises no more than 80, 100, 150 or 200 gene products. In some embodiments, determining the levels of each gene product in the sample comprises determining the Unique Molecular Identifier (UMI) counts for each gene product.

As used herein, the term “unique molecular identifier” or “UMI” refers to a short nucleic acid molecule (typically about 5 to about 30, more typically 8-12 bases in length and up to about 100 bases in length) providing a unique identifier tag for each macromolecule or binding agent to which it is linked. UMIs are a subtype of nucleic acid barcodes, which, when incorporated into nucleic acid molecules in accordance with the invention, can be used to correct for subsequent amplification bias by directly counting UMIs that are sequenced after amplification. The design, incorporation and application of UMIs is described, for example, in WO 2012/142213.

In another embodiment, step (ii) comprises normalizing the level of each gene product in the sample to the levels of other gene products in the sample. In another embodiment determining the levels of each gene product in the sample further comprises normalizing the level of each UMI in said sample to the levels of other UMIs in said sample. In another embodiment, the expression of each gene product in the sample is normalized by dividing its level by the sum of levels (e.g. UMIs) of all genes in said sample, thereby determining the sum-normalized expression for each gene product in the sample. In another embodiment, the expression of each gene product is further normalized to its maximal expression in other (e.g. positive and negative control) fecal samples. In another embodiment, the sum-normalized expression is divided by its maximal expression in fecal samples, thereby determining the max-normalized expression for each gene product in the sample. For example, determination of max-normalized UMIs as described above provides a standardized numerical value between 0 and 1 for each gene product.

In another embodiment, obtaining the transcriptomic signature of the sample with respect to the plurality of gene products comprises determining the collective (e.g. summed) expression levels of the plurality of gene products in said sample. In another embodiment, obtaining the transcriptomic signature of the sample with respect to the plurality of gene products comprises determining the sum of max-normalized expression levels for each gene product in the sample. In other embodiments, step (ii) comprises determining the transcriptomic signature of the sample with respect to a first plurality of gene products and a second plurality of gene products. For example, each of the first and second plurality of gene products may be characteristic of a distinct inflammatory state, level or patient population, e.g. with respect to disease severity or location.

### Classification and Analysis

According to some embodiments, the invention uses supervised classification algorithms to compare the levels of gene products in the sample to their corresponding levels in a control sample.

Supervised classifiers are prediction tools based on learning from examples of labeled data. A supervised classification algorithm is a form of learning and pattern recognition algorithm, in which labeled data, consisting of input (typically vector)-output (correct classification) pairs, is used to train the classifier. Through the training process, a classification function is inferred from labeled training data. The classification function can then be used for classifying new examples, thereby correctly determining the class labels for unseen instances.

In certain additional embodiments, step (iii) comprises comparing the transcriptomic signature of said sample to a control transcriptomic signature to said plurality of gene products (e.g. the signature corresponding to a fecal sample obtained from a healthy control to the same plurality of gene products). In other embodiments, step (ii) comprises determining the transcriptomic signatures of the sample with respect to a first plurality of gene products and a second plurality of gene products, and step (iii) comprises comparing the transcriptomic signatures of said sample with respect to the first plurality of gene products and the second plurality of gene products to control transcriptomic signatures with respect to said first plurality of gene products and said second plurality of gene products.

In another embodiment, step (iii) may comprise dividing the sum of max-normalized expression levels for each of the first plurality of gene products in the sample obtained from the subject by the collective sum of max-normalized expression levels for each of the first plurality and second plurality of gene products in the sample obtained from the subject. In another embodiment step (iii) may further comprise dividing the sum of max-normalized expression levels for each of the first plurality of gene products in a control sample by the collective sum of max-normalized expression levels for each of the first plurality and second plurality of gene products in the control sample. In another embodiment step (iii) may further comprise determining thresholds for assigning the sample to a particular diagnostic state based on the values obtained for the sample and control samples. For example, thresholds are typically determined so as to provide sensitivity and specificity of at least 50%, and more typically, at least 70%, at least 80%, at least 90%, at least 95% and up to 100%.

In other embodiments, other supervised classification algorithms including, but not limited to, support vector machines (SVM) or decision trees may be used. Additional supervised classification algorithms include, without limitation, gradient boosted trees, random forest, regularized regression, multiple linear regression (MLR), principal component regression (PCR), partial least squares (PLS), discriminant function analysis (DFA) including linear discriminant analysis (LDA), nearest neighbor, artificial neural networks, multi-layer perceptrons (MLP), generalized regression neural network (GRNN), and combinations thereof.

For example, in machine learning, support vector machines (SVMs, also support vector networks) are supervised learning models with associated learning algorithms that analyze data and recognize patterns, used for classification and regression analysis. Given a set of training examples, each marked as belonging to one of two categories, an SVM training algorithm builds a model that assigns new examples into one category or the other, making it a non-probabilistic binary linear classifier. An SVM model is a representation of the examples as points in space, mapped so that the examples of the separate categories are divided by a clear gap that is as wide as possible. New examples are then mapped into that same space and predicted to belong to a category based on which side of the gap they fall on.

Decision tree learning uses a decision tree as a predictive model which maps observations about an item to conclusions about the item's target value. It is one of the predictive modelling approaches used in statistics, data mining and machine learning. Tree models where the target variable can take a finite set of values are called classification trees. In these tree structures, leaves represent class labels and branches represent conjunctions of features that lead to those class labels.

LDA and the related Fisher's linear discriminant are methods used in statistics, pattern recognition and machine learning to find a linear combination of features which characterizes or separates two or more classes of objects or events. The resulting combination may be used as a linear classifier or, more commonly, for dimensionality reduction before later classification.

In various embodiments, the control transcriptomic signature may correspond to at least one control individual, a panel of control samples from a set of control individuals, or a stored set of data of control individuals. In some embodiments, control individuals may be healthy control individuals. In other embodiments, control individuals may correspond to individuals with a known severity or location of GI inflammation, and/or with known types of GI diseases associated therewith.

Accordingly, the principles of the invention provide for improved diagnostics using fecal samples based on host transcriptome analyses. Thus, embodiments of the invention utilize transcriptomic signatures and classifiers reflecting human gene expression that are not substantially affected by alterations in microbial gene expression.

### Gastrointestinal Inflammation

The biological response of body tissues to injury, infection or irritation is typically characterized by inflammation, an immune reaction in which a cascade of cellular and microvascular events serves to eradicate the infection, remove damaged tissue and generate new tissue. During this process, elevated permeability in microvessels allows neutrophils and mononuclear cells to leave the intravascular compartment, and perform various anti-microbial activities to eradicate the injury. As used herein, the term “Gastrointestinal inflammation” (or GI inflammation) refers to inflammation of a mucosal layer of the gastrointestinal tract, such as, for example, the upper gastrointestinal tract (e.g., esophagus, stomach, and/or duodenum), or the lower gastrointestinal tract (e.g., bowel such as small and/or large intestines). GI inflammation may be chronic or acute. Acute inflammation is generally characterized by a short time of onset and infiltration or influx of neutrophils. Chronic inflammation is generally characterized by a relatively longer period of onset and infiltration or influx of mononuclear cells. Chronic inflammation can also be characterized by periods of spontaneous remission and spontaneous occurrence (also referred to as “flares”). GI inflammation may be involved in the etiology and/or pathology of various GI conditions (herein referred to as “GI inflammatory conditions”), including, but not limited to, IBD and specific forms thereof as discussed herein, as well as other immune or inflammatory conditions such as various forms of colitis (e.g., ulcerative, granulomatous, ischemic, radiation-induced, infectious), ileitis and gastritis.

As used herein, the term “active GI inflammation” relates to the presence of an inflammatory pathology in the GI tract of a subject such as a flare or a state clinically determined as endoscopic inflammation or histological inflammation, and excludes patients afflicted with inflammatory conditions that are in a state of remission (inactive inflammation) at the time of sample collection.

In some embodiments, the subject is suspected of having colonic GI inflammation or terminal ileal GI inflammation. The terms “colonic inflammation” and “terminal ileal GI inflammation” indicate inflammation involving at least the colon or the terminal ileum, respectively. A subject may be suspected of having these conditions based on common symptoms or signs such as abdominal pain or cramping, diarrhea or constipation, and/or rectal bleeding.

Inflammatory bowel disease (IBD), which includes Crohn disease (CD) and ulcerative colitis (UC), is a relapsing and remitting condition characterized by chronic inflammation at various sites in the gastrointestinal tract, which results in diarrhea and abdominal pain. Although CD and UC are similar, they can be distinguished in most cases by their symptoms and/or histology. For example, in UC, colonic involvement is usually left-sided, whereas in CD, colonic involvement is usually right-sided. In another example, UC generally involves gross rectal bleeding whereas in CD gross rectal bleeding is rare, except in 75-85% of cases of Crohn colitis. However, diagnosis is often invasive and may be complicated or delayed, with certain patients remaining inappropriately diagnosed. For example, about 10% of colitis cases are not initially distinguishable and are termed unclassified; if a surgical pathologic specimen cannot be classified, it is termed indeterminate colitis.

In some embodiments, CD may involve the ileum alone (e.g. Crohn's ileitis or terminal ileal CD). In other embodiments, the CD involves the colon, with or without the terminal ileum. For example, colonic CD, Crohn's Colitis or granulomatous colitis are characterized by colonic involvement. In some embodiments, the CD involves both the ileum and the colon (e.g. ileocolitis or colonic CD with terminal ileal involvement). In other embodiments, the CD involves the colon alone, and is conveniently referred to herein as “colonic CD that does not involve the terminal ileum”.

The term “Simple Endoscopic Score in Crohn's Disease” (SES-CD or SESCD) refers to a standardized endoscopic measurement scale that grades the following variables: ulcer size (diameter 0.1-0.5 cm, 0.5-2 cm, or >2 cm), proportion of ulcerated surface (<10%, 10%-30%, or >30%), proportion of the surface area affected by any disease lesion (<500%, 50%-75%, or >75%), and stenosis (single, multiple, whether the colonoscopy passes through the narrowing), in the ileum, right colon, transverse colon, left colon, or rectum. Each variable is scored from 0-3, and a total score is calculated as a sum of all the variables in each intestinal segment, as follows: 0-no inflammation/remission, 1-mild inflammation, 2-moderate inflammation, and 3-severe inflammation.

The Mayo Score is a standardized endoscopic score used for evaluating the severity of UC. It includes clinical and endoscopic variables: stool frequency, bleeding, inflammatory activity on sigmoidoscopy, overall physician assessment and daily activities of the patient are assessed. The score ranges from 0-no inflammation/remission, 1-mild inflammation, 2-moderate inflammation, and 3-severe inflammation.

In other embodiments, the GI inflammatory condition is associated with acute bacterial inflammation (e.g. infectious colitis or infectious ileitis). In another embodiment, the GI inflammation is associated with diverticulitis. In other embodiments, the GI inflammatory condition is colonic diverticulitis. Typically, the GI inflammation is not associated with celiac disease. In some embodiments, the GI inflammation is not associated with colorectal cancer and/or colonic polyps. Accordingly, advantageous methods of the invention are referred to with the proviso that the subject had not been diagnosed with celiac disease, colorectal cancer and/or colonic polyps.

### Diagnostic and Analytical Methods

According to advantageous embodiments, the plurality of human gene products to be used in accordance with the methods and kits of the invention is selected from the group consisting of:
AC007192.1, ACSL1, ADGRG3, ALDOB, ALOX5AP, ALPL, AMN, ANP32E, AOC1, APBB1IP, APOA1, APOA4, APOB, APOC3, AQP9, ARHGAP26, ARHGAP30, ARRB2, BCL6, BID, CAPZA1, CASP5, CCL3, CCL3L3, CCL4, CCL4L2, CCR1, CD44, CD53, CD83, CDHR2, CDHR5, CLEC2B, CLEC4E, CLEC7A, CMTM2, CREB3L3, CREM, CSF3R, CXCL8, CXCR2, CYP3A4, CYTH1, CYTH4, DDX21, DEFA1B, DEFA3, DEFA5, DEFA6, DNAJA1, DPEP1, EIF3J, EPS8L1, ERICH1, EWSR1, FABP2, FABP6, FAM129A, FCER1G, FCGR2A, FCGR3A, FCGR3B, FNBP1, FPR1, FYB1, GOS2, GBP1, GBP4, GBP5, GDF5OS, GMFG, GNA11, GPR65; GUCA2A, GUCA2B, HCAR2, HCAR3, HNRNPA2B1, ICAM1, IFI16, IFIT2, IFIT3, IFITM2, IGSF6, IL1B, IL1R2, IL1RAP, IL1RN, ILF3, ITGAX, ITSN2, KCNJ15, KCNK6, KIAA0825, KIAA1109, LCP1, LCP2, LILRB3, LRRK2, LSP1, LYN, MAVS, MEP1A, METAP2, MNDA, MTRNR2L1, MTTP, NAMPT, NBN, NCF2, NCL, OLR1, OSM, PDE4B, PFKFB3, PHACTR1, PHIP, PLAU, PLCB2, PLCD3, PLEK, PPIF, PROK2, PTGS2, RAPGEF6, RCC1, REG1A, REG1B, RHOH, S100A4, S100A9, SAMSN1, SELENOP, SELL, SH3BP5, SI, SIPA1L2, SLC15A1, SLC2A3, SLC5A1, SMIM24, SNX10, SOCS3, SOD2, SP140, SRGN, SUPT6H, SYNE2, TANK, TET3, TLR2, TM4SF5, TMEM154, TNFAIP6, TRA2B, TREM1, UTRN, VNN2, WDR66, YTHDC1, ZEB2, ZFC3H1, ZNF267, and ZNF511-PRAP1.

In various embodiments, said plurality of gene products comprises at least 11 or 12 and typically at least 20 of said gene products, more typically at least 35 of said gene products, e.g. 35-75, 35-55, or 50-75 of said gene products (RNA transcripts). In another embodiment, said plurality of gene products comprises, or consists of, at least 12, 15, 20, 25, 30, 35, 40 or 45 of the gene products in a transcriptomic signature identified herein. In another embodiment said plurality of gene products comprises up to 75, 100, 125, 150, 175 or 200 gene products (e.g. 11-75, 35-100 or 50-150 gene products selected from Table 1 or Table 4). Each possibility represents a separate embodiment of the invention.

In some embodiments, the plurality of gene products is selected from Table 1. In other embodiments, the plurality of gene products is selected from Table 4. In some embodiments, the plurality of gene products is selected from Table 1 and Table 4. In other embodiments, the plurality of gene products is as disclosed herein.

In some embodiments, methods in accordance with the principles of the invention are used for analyzing fecal samples. In various embodiments, methods in accordance with the principles of the invention may be used for various diagnostic and prognostic purposes, including for evaluating, determining or monitoring the existence, severity and/or location of GI inflammation, wherein each possibility represents a separate embodiment of the invention. In another embodiment, there are provided diagnostic classifiers for analyzing fecal samples and for evaluating, determining or monitoring the existence, severity and/or location of GI inflammation, wherein each possibility represents a separate embodiment of the invention.

In some embodiments, the method is used for determining the location of GI inflammation. In one embodiment the subject is afflicted with IBD. In another embodiment the subject is diagnosed with IBD. In another embodiment the subject is suspected of having IBD. In another embodiment the subject is afflicted with CD. In another embodiment the subject is diagnosed with CD. In another embodiment the subject is suspected of having CD. In another embodiment, the method is used for identifying colonic CD. In another embodiment the method is used for identifying terminal ileal CD. In another embodiment the method is used for differentiating colonic CD from terminal ileal CD.

In some embodiments relating to determining the location of GI inflammation (e.g. for identifying colonic CD), the plurality of gene products is selected from the group consisting of Group A gene products. In another embodiment said plurality of gene products comprises or consists of Group A gene products. In yet other embodiments relating to determining the location of GI inflammation, the plurality of gene products is selected from Table 4.

In other embodiments relating to determining the location of GI inflammation (e.g. for identifying terminal ileal CD), the plurality of gene products is selected from the group consisting of Group B gene products. In other embodiments, the plurality of antigens comprises, or consists of, Group B gene products.

In other embodiments relating to determining the location of GI inflammation (e.g. for differentiating colonic CD from terminal ileal CD), the first plurality of gene products is selected from the group consisting of Group A gene products, and the second plurality of gene products is selected from the group consisting of Group B gene products. In another embodiment, the first plurality of gene products comprises or consists of Group A gene products, and the second plurality of gene products comprises or consists of Group B gene products.

Thus, in another embodiment, there is provided a method of determining the location of GI inflammation, comprising:
i. providing a fecal RNA sample of a subject afflicted with, or suspected of having, GI inflammation,
ii. determining the levels in the sample of a plurality of human gene products, thereby obtaining the transcriptomic signature of the sample with respect to the plurality of gene products, and
iii. comparing the transcriptomic signature of said sample to a control transcriptomic signature by a supervised classification algorithm,
   wherein said plurality of gene products is selected from the group consisting of: a first plurality of gene products comprising Group A gene products, and/or a second plurality of gene products comprising Group B gene products. In another embodiment the outcome of the comparison is indicative of the location of GI inflammation in said subject.

In another embodiment, said subject is suspected of having colonic GI inflammation or terminal ileal GI inflammation. In another embodiment said subject is suspected of having colonic CD or terminal ileal CD. In another embodiment, step (ii) comprises determining the transcriptomic signatures of the sample with respect to the first plurality of gene products and the second plurality of gene products. In another embodiment, the control transcriptomic signature comprises at least one of: a control transcriptomic signature with respect to the plurality of gene products corresponding to colonic CD that does not involve the terminal ileum, and a control transcriptomic signature with respect to the plurality of gene products corresponding to terminal ileal CD.

In another embodiment, the method comprises comparing said transcriptomic signatures to a first control transcriptomic signature corresponding to colonic CD that does not involve the terminal ileum and to a second control transcriptomic signature corresponding to terminal ileal CD. In another embodiment, said transcriptomic signature reflects the collective levels of said first plurality of gene products in said sample as compared to the collective levels of said second plurality of gene products in said sample, and wherein:
the higher the enhancement of the collective levels of said first plurality of gene products in said sample as compared to the collective levels of said second plurality of gene products in said sample, the higher the probability that said subject is afflicted with colonic CD that does not involve the terminal ileum, and/or
the higher the enhancement of the collective levels of said second plurality of gene products in said sample as compared to the collective levels of first plurality of gene products in said sample, the higher the probability that said subject is afflicted with terminal ileal CD.

In another embodiment, a transcriptomic signature characterized by significant enhancement of the collective levels of said first plurality of gene products in said sample as compared to a control transcriptomic signature corresponding to terminal ileal CD indicates that said subject is afflicted with colonic CD, and/or

a transcriptomic signature characterized by significant enhancement of the collective levels of said second plurality of gene products in said sample as compared to a control transcriptomic signature corresponding to colonic CD that does not involve the terminal ileum indicates that said subject is afflicted with terminal ileal CD.

As used herein, “significantly enhanced” and “significantly reduced” levels (or significant enhancement/reduction in said levels) refer to statistically significant enhancement/reduction, respectively.

In another embodiment, a transcriptomic signature characterized by significant enhancement of the collective levels of said first plurality of gene products in said sample as compared to the collective levels of said second plurality of gene products in said sample indicates that said subject is afflicted with colonic CD that does not involve the terminal ileum, and/or a transcriptomic signature characterized by significant enhancement of the collective levels of said second plurality of gene products in said sample as compared to the collective levels of said first plurality of gene products in said sample indicates that said subject is afflicted with terminal ileal CD.

In another embodiment, there is provided a method of determining the location of GI inflammation, comprising:
i. providing a fecal RNA sample of a subject afflicted with, or suspected of having, GI inflammation,
ii. determining the levels in the sample of a plurality of human gene products, thereby obtaining the transcriptomic signature of the sample with respect to the plurality of gene products, and
iii. comparing the transcriptomic signature of said sample to a control transcriptomic signature by a supervised classification algorithm,
   wherein said plurality of gene products is selected from Table 4, and wherein the outcome of the comparison is indicative of the location of GI inflammation in said subject.

In another embodiment the method further comprises providing said subject with a treatment suitable to the determined location of GI inflammation in said subject.

In another embodiment there is provided a method of analyzing a stool sample, comprising:
i. providing a fecal RNA sample of a subject afflicted with, or suspected of having, colonic GI inflammation or terminal ileal GI inflammation,
ii. determining the levels in the sample of a plurality of human gene products selected from Table 4, thereby obtaining the transcriptomic signature of the sample with respect to the plurality of gene products.

In another embodiment the fecal RNA sample has been obtained from a solid fecal (stool) sample by a method as disclosed herein.

In another embodiment, the method is used for determining the severity of GI inflammation. In one embodiment the subject is afflicted with IBD. In another embodiment the subject is diagnosed with IBD. In another embodiment the subject is suspected of having IBD. In another embodiment the subject is afflicted with CD. In another embodiment the subject is diagnosed with CD. In another embodiment the subject is suspected of having CD. In another embodiment the method is used for predicting endoscopic severity characterized by MAYO/SESCD score of at least 2. In another embodiment the method is used for predicting endoscopic severity characterized by MAYO/SESCD score lower than 2.

In some embodiments relating to determining the severity of GI inflammation (for example predicting endoscopic severity characterized by MAYO/SESCD score of at least 2), the plurality of gene products is selected from the group consisting of Group C gene products. In other embodiments, the plurality of antigens comprises, or consists of, Group C gene products.

In other embodiments relating to determining the severity of GI inflammation (for example predicting endoscopic severity characterized by MAYO/SESCD score lower than 2), the plurality of gene products is selected from the group consisting of Group D gene products. In other embodiments, the plurality of antigens comprises, or consists of, Group D gene products.

In other embodiments relating to determining the severity of GI inflammation, the first plurality of gene products is selected from the group consisting of Group C gene products, and the second plurality of antigens is selected from the group consisting of Group D gene products.

In other embodiments relating to determining the severity of GI inflammation, the first plurality of gene products comprises or consists of Group C gene products, and the second plurality of antigens comprises or consists of Group D gene products.

In another embodiment, there is provided a method of determining the severity of GI inflammation, comprising:
i. providing a fecal RNA sample of a subject afflicted with, or suspected of having, GI inflammation,
ii. determining the levels in the sample of a plurality of human gene products, thereby obtaining the transcriptomic signature of the sample with respect to the plurality of gene products, and
iii. comparing the transcriptomic signature of said sample to a control transcriptomic signature by a supervised classification algorithm,
wherein said plurality of gene products is selected from the group consisting of: a first plurality of gene products comprising (or selected from) Group C gene products; and/or a second plurality of gene products comprising (or selected from) Group D gene products. In another embodiment the outcome of the comparison is indicative of the severity of GI inflammation in said subject.

In another embodiment, step (ii) comprises determining the transcriptomic signature of the sample with respect to the first plurality of gene products of Group C and the second plurality of gene products of Group D. In another embodiment, the control transcriptomic signature comprises control transcriptomic signature with respect to the plurality of gene products corresponding to at least one of: a healthy control (not afflicted with GI inflammation and not diagnosed with a GI inflammatory disease), inactive GI inflammation (e.g. SESCD/Mayo endoscopic score 0), mild GI inflammation (e.g. SESCD/Mayo endoscopic score 1), moderate GI inflammation (e.g. SESCD/Mayo endoscopic score 2), and severe GI inflammation (e.g. SESCD/Mayo endoscopic score 3). In another embodiment, the control transcriptomic signature comprises control transcriptomic signature with respect to the plurality of gene products corresponding to at least one of: a healthy control, GI inflammation characterized by an endoscopic score of at least 2, and GI inflammation characterized by an endoscopic severity lower than 2, wherein said endoscopic severity is determined according to the Mayo and/or Simple Endoscopic Score (SESCD) scores. In another embodiment said transcriptomic signature reflects the collective levels of said first plurality of gene products in said sample as compared to the collective levels of said second plurality of gene products in said sample, and wherein the higher the enhancement of said collective levels of said first plurality of gene products in said sample as compared to said collective levels of said second plurality of gene products in said sample, the higher the predicted severity of GI inflammation in said subject.

In another embodiment, a transcriptomic signature characterized by significant enhancement of the collective levels of said first plurality of gene products (of Group C) in said sample as compared to the collective levels of said second plurality of gene products (of Group D) in said sample indicates that said subject is afflicted with GI inflammation characterized by an endoscopic score of at least 2, and/or

a transcriptomic signature characterized by significant enhancement of the collective levels of said second plurality of gene products in said sample as compared to the collective levels of said first plurality of gene products in said sample indicates that said subject is afflicted with GI inflammation characterized by an endoscopic severity lower than 2, wherein said endoscopic severity is determined according to the Mayo and/or SESCD scores.

In other embodiments, the method is used for identifying GI inflammation in the subject (e.g. an active state of inflammation, such as a flare or a state of chronic inflammation clinically determined as endoscopic inflammation or histological inflammation). In one embodiment the subject is afflicted with IBD. In another embodiment the subject is diagnosed with IBD. In another embodiment the subject is suspected of having IBD (or a form thereof such as CD or UC). In various embodiments, said plurality of gene products is selected from Group E, F, G, H, I and/or J gene products as described herein, wherein each possibility represents a separate embodiment of the invention. In another embodiment said plurality of gene products is selected from Group E, F, G, H and/or I gene products.

In another embodiment, the plurality of gene products is selected from the group consisting of Group E gene products. In another embodiment the plurality of gene products is selected from the group consisting of Group F gene products. In another embodiment the plurality of gene products is selected from the group consisting of: DEFA3, DEFA1B, CD53, FCER1G, ADGRG3, CD83, CMTM2, IFIT2, HCAR3, CCL4L2, CCL3L3, GMFG, NBN, SOD2, OLR1, CCL4, IFIT3, CREM, GBP4 and GPR65. In another embodiment the plurality of gene products is selected from the group consisting of: DEFA3, DEFA1B, CD53, FCER1G, ADGRG3, CD83, CMTM2, IFIT2, HCAR3, and CCL4L2.

In another embodiment relating to for identifying GI inflammation, the plurality of gene products comprises, or consists of, Group E, F, G, H, I and/or J gene products, wherein each possibility represents a separate embodiment of the invention. In another embodiment relating to for identifying GI inflammation, the plurality of gene products comprises, or consists of Group E gene products. In another embodiment relating to for identifying GI inflammation, the plurality of gene products comprises, or consists of, Group F gene products. In another embodiment said plurality of gene products comprises Group E, F, G, H and/or I gene products, and further comprises a plurality of gene products selected from Group J.

In another embodiment there is provided a method for identifying GI inflammation, comprising:
i. providing a fecal RNA sample of a subject suspected of having GI inflammation,
ii. determining the levels in the sample of a plurality of human gene products, thereby obtaining the transcriptomic signature of the sample with respect to the plurality of gene products, and
iii. comparing the transcriptomic signature of said sample to a control transcriptomic signature by a supervised classification algorithm,
   wherein said plurality of gene products comprises Group E gene products, or Group F gene products.

In another embodiment the outcome of the comparison is indicative of the presence or absence of GI inflammation in said subject. In another embodiment the control transcriptomic signature corresponds to a healthy control, and a transcriptomic signature characterized by significant enhancement of the collective levels of said plurality of gene products in said sample as compared to said healthy control indicates that said subject is afflicted with GI inflammation.

In another embodiment (wherein the gene products are selected from Group E, F, G, H and/or I gene products), the control transcriptomic signature corresponds to a healthy control, and wherein a transcriptomic signature characterized by significant enhancement of the collective levels of said plurality of gene products in said sample as compared to said healthy control indicates that said subject is afflicted with GI inflammation

In another embodiment (wherein the gene products further comprise Group J gene products), the control transcriptomic signature corresponds to a healthy control, and wherein a transcriptomic signature characterized by significant enhancement of the collective levels of gene products selected from Group E, F, G, H and/or I, and by a significant reduction of the collective levels of gene products selected from Group J, in said sample as compared to said healthy control, indicates that said subject is afflicted with GI inflammation.

For example, for the generation of a diagnostic classifier using both upregulated and downregulated genes, expression patterns from both categories may be integrated to improve sensitivity and specificity, e.g. by calculating a composite score that combines the levels of upregulated and downregulated genes, or using statistical methods such as logistic regression, support vector machine, machine learning models or other suitable algorithms as disclosed herein. By means of non-limitative examples, a ratio of upregulated to downregulated gene expression may be calculated (e.g., total UMIs from upregulated genes divided by the sum of downregulated ones and upregulated ones), which can serve as an inflammation index. This ratio can be used to set a threshold for classification purposes. Alternatively, normalization techniques such as z-scores, fold-change calculations, or machine learning classifiers can be used to weigh both groups appropriately without relying on fixed ratios.

While the potential involvement of some of the genes described herein in various pathologies has been suggested, the invention provides for the first time accurate classifiers providing for comprehensive diagnostic and prognostic evaluation of GI inflammation in a subject in need thereof. For example, it is demonstrated herein that by comparing the relative levels of gene products (such as Group A as compared to Group B, or Group C as compared to Group D), and/or by considering relevant control transcriptomic signatures (e.g. corresponding to a particular intestinal location or degree of inflammatory severity), it is possible to obtain an accurate and thorough evaluation of the status of the subject. Thus, the invention facilitates non-invasive evaluation, using a clinically-manageable number of gene products, of GI inflammation in a subject, in a manner that facilitates early assignment of the most suitable and effective treatment to said subject. Conveniently, the use of the classifiers of the invention (including comparison of discrete gene product groups and assignment of relevant controls as disclosed herein), further provides for improved diagnostic capacity while reducing the total number of gene products to be assayed (which may optionally be shared among classifiers).

### Diagnostic Kits

In other embodiments, the invention is directed to articles of manufacture (such as diagnostic kits) which may be used to facilitate the methods of the invention. Thus, in another aspect, there is provided a diagnostic kit, comprising means for specifically determining and quantifying the levels of a plurality of human gene products as disclosed herein in a fecal RNA sample. In various embodiments, the plurality of human gene products comprises or consists of: Group A gene products, Group B gene products, Group C gene products, Group D gene products, Group E gene products; and/or Group F gene products. In various other embodiments, the plurality of human gene products comprises or consists of: Group G gene products, Group H gene products, Group I gene products and/or Group J gene products. In yet other embodiments, said plurality of human gene products comprises or consists of any one of gene product Groups A to J as disclosed herein, or subsets or combinations thereof. In other embodiments, said plurality of human gene products is as disclosed herein. Each possibility represents a separate embodiment of the invention.

As used herein, means for specifically determining and quantifying the levels of a plurality of gene products denotes structure-specific reagents (such as primers, probes or antibodies) which facilitate the assessment of the amount of the particular gene products in question in a sample. Reagents facilitating the evaluation of the amounts of DNA or RNA in a non-specific manner (that does not allow an evaluation of the level of a particular gene product as compared to other gene products) are not considered to facilitate “specific” determination and/or quantification. Accordingly, reagents such as buffers or enzymes are not considered to allow specific determination and quantification of the levels of gene products unless accompanied by sequence-specific reagents such as primers or probes.

In some embodiments, the means for specifically determining and quantifying the levels of a plurality of gene products may comprise primers or probes for facilitating specific quantification of the gene products. In some embodiments, said primers or probes may be attached to a surface so as to form a diagnostic article of manufacture, e.g. a gene expression array or chip. In an exemplary embodiment the means comprise quantitative polymerase chain reaction (qPCR) primers directed to said plurality of human gene products. In some embodiments, the means for specifically determining and quantifying the levels of a plurality of gene products comprise primers or probes to be used in an assay disclosed herein for determining and quantifying the levels of gene products, including, but not limited to droplet digital PCR (ddPCR) and targeted sequencing (also referred to herein as targeted transcriptomics).

In some embodiments, the diagnostic kits of the invention further comprise qPCR reagents. For example, the kits may include one or more of a Taq enzyme premix system (Taq enzyme, buffer, and dNTP), primers and probes of target genes for qPCR, a reagent for quality control of an activity of a Taq enzyme, reagents for plotting a reference curve and instructions for parameter set and procedure.

In another embodiment, the kit further comprising means for providing a fecal RNA sample and/or means for comparing the levels of said plurality of human gene products in the sample to their levels in a control fecal RNA sample. Each possibility represents a separate embodiment of the invention.

In some embodiments, means for providing a fecal RNA sample may contain a container for collecting a fecal sample, a cryopreservation reagent such as an RNA preserving agent, or both. In a particular embodiment, the means for providing the fecal RNA sample comprise contain a container for collecting a fecal sample which contains a solution comprising 20-30 mM sodium citrate, 5-15 mM EDTA, 60-80 g ammonium sulfate per 100 ml solution, and has a pH of 5-6, e.g. an RNAlater solution. In some embodiments, the container may conveniently contain an amount of RNA preservation reagent at a 5-15-fold (e.g. 10-fold) excess to the volume of the sample to be collected. For example, without limitation, the container may include 3-4 ml of an RNA preservation reagent (e.g. RNAlater) for a specimen of about 0.5 ml stool.

Additionally or alternatively, means for providing a fecal RNA sample may contain means for RNA isolation and/or processing, e.g. RNAse H or other reagents for selectively depleting microbial rRNA, as disclosed herein.

In various embodiments, means for comparing the levels of said plurality of human gene products in the sample to their levels in a control fecal RNA sample, may contain control fecal RNA samples, control values corresponding to the levels of sad gene products in control fecal RNA samples, stored sets of data corresponding to control fecal RNA samples (conveniently stored on a storage device), or a computer implemented storage device comprising a suitable supervised classification algorithm and/or suitable control sets of data.

In another embodiment, said plurality of human gene products is as set forth in a. and/or b. as defined with respect to the kit above, and the kit is for use in determining the location of GI inflammation in a subject and/or comprises instructions for use in determining the location of GI inflammation in a subject. Each possibility represents a separate embodiment of the invention.

In another embodiment said plurality of human gene products is as set forth in c. and/or d. as defined with respect to the kit above, and the kit is for use in determining the severity of GI inflammation in a subject and/or comprises instructions for use in determining the severity of GI inflammation in a subject. Each possibility represents a separate embodiment of the invention.

In another embodiment said plurality of human gene products is as set forth in e. and/or f. as defined with respect to the kit above, and the kit is for use in determining the presence or absence of GI inflammation in a subject and/or comprises instructions for use in determining the presence or absence of GI inflammation in a subject. Each possibility represents a separate embodiment of the invention.

In some embodiments, the kit may further contain means for isolation, extraction or derivation of RNA by a suitable method.

### RNA Isolation and Processing and Evaluation of Gene Product Levels

The following exemplary processes and reagents for RNA isolation, extraction and derivation may be used in the kits and methods of the invention.

Isolating RNA from a biological sample generally includes treating a biological sample in such a manner that the RNA present in the sample is extracted and made available for analysis. For example, phenol based extraction methods are single-step RNA isolation methods based on Guanidine isothiocyanate (GITC)/phenol/chloroform extraction require much less time than traditional methods (e.g. CsCl2 ultracentrifugation). Many commercial reagents (e.g. Trizol, RNAzol, RNAWIZ) are based on this principle. The entire procedure can be completed within an hour to produce high yields of total RNA.

Additional methods for RNA isolation include e.g. silica gel-based purification methods and/or oligo-dT based affinity purification of mRNA. In silica gel-based purification, RNeasy is a purification kit marketed by Qiagen. It uses a silica gel-based membrane in a spin-column to selectively bind RNA larger than 200 bases. The method is quick and does not involve the use of phenol. In addition, due to the low abundance of mRNA in the total pool of cellular RNA, reducing the amount of rRNA and tRNA in a total RNA preparation greatly increases the relative amount of mRNA. The use of oligo-dT affinity chromatography to selectively enrich poly (A)+RNA has been practiced for over 20 years. The result of the preparation is an enriched mRNA population that has minimal rRNA or other small RNA contamination. mRNA enrichment is essential for construction of cDNA libraries and other applications where intact mRNA is highly desirable. The original method utilized oligo-dT conjugated resin column chromatography and can be time consuming. Recently more convenient formats such as spin-column and magnetic bead based reagent kits have become available.

The sample may also be processed prior to carrying out the diagnostic methods of the present invention. Processing of the sample may involve one or more of: filtration, distillation, centrifugation, extraction, concentration, dilution, purification, inactivation of interfering components, addition of reagents, and the like. As disclosed herein, fecal RNA samples in accordance with the invention may be subjected to selective depletion of microbial ribosomal RNA (rRNA). Thus, suitable reagents for facilitating such processes as described herein may be used.

After obtaining the RNA sample, cDNA may be generated therefrom. For synthesis of cDNA, template mRNA may be obtained directly from lysed cells or may be purified from a total RNA or mRNA sample. The total RNA sample may be subjected to a force to encourage shearing of the RNA molecules such that the average size of each of the RNA molecules is between 100-300 nucleotides, e.g. about 200 nucleotides. To separate the heterogeneous population of mRNA from the majority of the RNA found in the cell, various technologies may be used which are based on the use of oligo (dT) oligonucleotides attached to a solid support. Examples of such oligo (dT) oligonucleotides include: oligo (dT) cellulose/spin columns, oligo (dT)/magnetic beads, and oligo (dT) oligonucleotide coated plates. According to another embodiment, long-read transcriptome sequencing is carried out, wherein the full-length RNA molecule is sequenced (i.e. from the 3′polyA tail to the 5′ cap).

Generation of single stranded DNA from RNA requires synthesis of an intermediate RNA-DNA hybrid. For this, a primer is required that hybridizes to the 3′ end of the RNA. Annealing temperature and timing are determined both by the efficiency with which the primer is expected to anneal to a template and the degree of mismatch that is to be tolerated.

The annealing temperature is usually chosen to provide optimal efficiency and specificity, and generally ranges from about 50° C. to about 80° C., usually from about 55° C. to about 70° C., and more usually from about 60° C. to about 68° C. Annealing conditions are generally maintained for a period of time ranging from about 15 seconds to about 30 minutes, usually from about 30 seconds to about 5 minutes.

According to a specific embodiment, the primer comprises a polydT oligonucleotide sequence. Preferably the polydT sequence comprises at least 5 nucleotides. According to another is between about 5 to 50 nucleotides, more preferably between about 5-25 nucleotides, and even more preferably between about 12 to 14 nucleotides.

Following annealing of the primer (e.g. polydT primer) to the RNA sample, an RNA-DNA hybrid is synthesized by reverse transcription using an RNA-dependent DNA polymerase. Suitable RNA-dependent DNA polymerases for use in the methods and compositions of the invention include reverse transcriptases (RTs). Examples of RTs include, but are not limited to, Moloney murine leukemia virus (M-MLV) reverse transcriptase, human immunodeficiency virus (HIV) reverse transcriptase, rous sarcoma virus (RSV) reverse transcriptase, avian myeloblastosis virus (AMV) reverse transcriptase, rous associated virus (RAV) reverse transcriptase, and myeloblastosis associated virus (MAV) reverse transcriptase or other avian sarcoma-leukosis virus (ASLV) reverse transcriptases, and modified RTs derived therefrom. Sec e.g. U.S. Pat. No. 7,056,716. Many reverse transcriptases, such as those from avian myeloblastosis virus (AMV-RT), and Moloney murine leukemia virus (MMLV-RT) comprise more than one activity (for example, polymerase activity and ribonuclease activity) and can function in the formation of the double stranded cDNA molecules. Additional components required in a reverse transcription reaction include dNTPS (dATP, dCTP, dGTP and dTTP) and optionally a reducing agent such as Dithiothreitol (DTT) and MnCl₂.

Following cDNA synthesis, the present inventors contemplate in some embodiments amplifying the cDNA (e.g. using a polymerase chain reaction-PCR, details of which are known in the art). Methods of analyzing the amount of RNA are known in the art and include e.g. Northern Blot analysis, RT-PCR analysis, RNA in situ hybridization stain, DNA microarray, DNA chips, oligonucleotide microarray, RNA sequencing and deep sequencing. Such methods may be used to determine the levels of gene products in accordance with embodiments of the invention.

In another embodiment, the method includes quantitative PCR (qPCR). qPCR generally refers to the PCR technique known as real-time quantitative polymerase chain reaction, quantitative polymerase chain reaction or kinetic polymerase chain reaction. This technique can simultaneously amplify and quantify target nucleic acids using PCR wherein the quantification is typically by virtue of an intercalating fluorescent dye or sequence-specific probes which contain fluorescent reporter molecules that are only detectable once hybridized to a target nucleic acid. Fluorescence can be monitored on each PCR cycle providing an amplification plot that allows a user to follow the reaction in real time. The amount of product detected at a certain point of the run is directly related to the initial amount of target in the sample.

Other methods for determining the levels of gene products include for example droplet digital PCR (ddPCR) and targeted sequencing. ddPCR is an emulsion PCR process that performs absolute quantitation by dividing nucleic acid samples into thousands of nanoliter-sized droplets and each droplet provide fundamentally the equivalent function as individual reaction. Targeted transcriptomics is a technique that uses specialized assays, such as multiplexed quantitative PCR, hybrid capture, or targeted RNA sequencing, to selectively analyze the expression levels of a predefined set of genes. It relies on custom-designed probes or primers that specifically hybridize to target transcripts, allowing for high specificity and sensitivity in gene quantification. This approach typically involves steps such as RNA extraction, reverse transcription to cDNA, target enrichment, and subsequent quantification using sequencing or amplification-based methods.

In another embodiment determining the level of at least one human gene product in the fecal RNA sample comprises RNA barcoding. In another embodiment determining the level of at least one human gene product in the fecal RNA sample comprises RNA sequencing. In another embodiment determining the level of at least one human gene product in the fecal RNA sample comprises RNA barcoding and sequencing.

In a particular embodiment, unique molecular identifiers (UMI) are used. Sequencing linker or a subtype of nucleic acid barcode may be used in a method that uses molecular tags to detect and quantify unique products (e.g., individual transcripts). A UMI may be used to distinguish effects through a single clone from multiple clones. The term “clone” as used herein may refer to a single mRNA or target nucleic acid to be sequenced. In one example embodiment, a random sequence of between 4 and 20 base pairs may be used, which may be designed such that assignment to the original can take place despite up to 4-7 errors during amplification or sequencing.

Unique molecular identifiers (UMIs) can be used, for example, to normalize samples for variable amplification efficiency. For example, in various embodiments, featuring a solid or semisolid support (for example a bead), to which nucleic acid barcodes (for example a plurality of barcodes sharing the same sequence) are attached, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support.

### Therapeutic Methods

According to some embodiments of the invention, the method further comprises informing the subject of the diagnosis. As used herein the phrase “informing the subject” refers to advising the subject that based on the diagnosis the subject should seek a suitable treatment regimen.

Optionally, once the diagnosis is confirmed using the methods described herein, the subject can be treated accordingly. IBD may be treated using anti-inflammatory drugs including, but not limited to corticosteroids (e.g. glucocorticoids such as budesonide (Uceris), prednisone (Prednisone Intensol, Rayos), prednisolone (Millipred, Prelone) and methylprednisolone (Medrol, Depo-Medrol)); 5-ASA drugs (aminosalicylates) including but not limited to balsalazide (Colazal), mesalamine (Apriso, Asacol HD, Canasa, Pentasa), olsalazine (Dipentum) and sulfasalazine (Azulfidine), immunomodulators including but not limited to methotrexate (Otrexup, Trexall, Rasuvo), azathioprine (Azasan, Imuran) and mercaptopurine (Purixan).

Other agents suitable for treating IBD include inhibitors of TNF-alpha (including but not limited to adalimumab (Humira), golimumab (Simponi) and infliximab (Remicade). Other biologics for treating IBD include certolizumab (Cimzia); natalizumab (Tysabri); ustekinumab (Stelara) and vedolizumab (Entyvio). Surgical interventions that can be recommended for treating IBD include strictureplasty to widen a narrowed bowel, closure or removal of fistulas, removal of affected portions of the intestines, for people with Crohn's disease and removal of the entire colon and rectum, for severe cases of UC.

In another embodiment, the methods of the invention may further contain a step of providing the subject with a treatment suitable for the outcome of diagnosis. For example, suitable therapeutic agents for colonic CD include e.g. Metronidazole, Budesonide, Ciprofloxacin, Metronidazole, Certolizumab pegol (CZP), Infliximab, Ustekinumab and anti-TNF alpha antibodies. In another example, suitable therapeutic agents for terminal ileal CD include e.g. Enteral Nutrition and Adalimumab. Exemplary treatments for mild IBD (e.g. mild UC) include e.g. mesalamine (5-ASA) (topical and/or oral), Topical steroids and Budesonide. Exemplary treatments for moderate IBD (e.g. moderate UC) include e.g. Systemic+topical steroids, Oral mesalamine, early biological therapy, azathioprinc (AZA), Thiopurines and Budesonide. Exemplary treatments for severe IBD (e.g. severe UC) include e.g. IV steroids, infliximab (IFX)+AZA, cyclosporin (CSA)+AZA, Surgery and biological therapy.

In another aspect, the invention provides a method of determining the location of gastrointestinal (GI) inflammation, comprising:
i. providing a fecal RNA sample of a subject afflicted with, or suspected of having, GI inflammation,
ii. determining the levels in the sample of a plurality of human gene products, thereby obtaining the transcriptomic signature of the sample with respect to the plurality of gene products, and
iii. comparing the transcriptomic signature of said sample to a control transcriptomic signature by a supervised classification algorithm,
   wherein said plurality of gene products is selected from the group consisting of: a first plurality of gene products comprising Group A gene products, and/or a second plurality of gene products comprising Group B gene products, wherein the outcome of the comparison is indicative of the location of GI inflammation in said subject, and wherein the method further comprises providing said subject with a treatment suitable for the determined location of GI inflammation in said subject, comprising:
Metronidazole, Budesonide, Ciprofloxacin, Metronidazole, Certolizumab pegol (CZP), Infliximab, Ustekinumab or anti-TNF alpha antibodies if said GI inflammation is determined to be located in the colon, and
Enteral Nutrition or Adalimumab if said GI inflammation is determined to be located in the terminal ileum.

In another aspect, the invention provides a method of determining the location of GI inflammation, comprising:
i. providing a fecal RNA sample of a subject afflicted with, or suspected of having, GI inflammation,
ii. determining the levels in the sample of a plurality of human gene products, thereby obtaining the transcriptomic signature of the sample with respect to the plurality of gene products, and
iii. comparing the transcriptomic signature of said sample to a control transcriptomic signature by a supervised classification algorithm,
   wherein said plurality of gene products is selected from Table 4, wherein the outcome of the comparison is indicative of the location of GI inflammation in said subject, and wherein the method further comprises providing said subject with a treatment suitable for the determined location of GI inflammation in said subject, comprising:
Metronidazole, Budesonide, Ciprofloxacin, Metronidazole, Certolizumab pegol (CZP), Infliximab, Ustekinumab or anti-TNF alpha antibodies if said GI inflammation is determined to be located in the colon, and
Enteral Nutrition or Adalimumab if said GI inflammation is determined to be located in the terminal ileum.

In another aspect, the invention provides a method of determining the severity of GI inflammation, comprising:
i. providing a fecal RNA sample of a subject afflicted with, or suspected of having, GI inflammation,
ii. determining the levels in the sample of a plurality of human gene products, thereby obtaining the transcriptomic signature of the sample with respect to the plurality of gene products, and
iii. comparing the transcriptomic signature of said sample to a control transcriptomic signature by a supervised classification algorithm,
   wherein said plurality of gene products is selected from the group consisting of: a first plurality of gene products comprising Group C gene products; and/or a second plurality of gene products comprising Group D gene products, wherein the outcome of the comparison is indicative of the severity of GI inflammation in said subject, and wherein the method further comprises providing said subject with a treatment suitable for the determined severity of GI inflammation in said subject, comprising:
topical mesalamine, oral mesalamine, topical steroids (such as topical prednisone or prednisolone) and/or Budesonide if the determined severity is mild,
systemic and topical steroids (e.g. oral prednisone or prednisolone), oral mesalamine, early biological therapy (such as Ozanimod, Upadacitinib, Tofacitinib, Risankizumab, Ustekinumab and/or Vedolizumab), azathioprine (AZA), Thiopurines and/or Budesonide if the determined severity is moderate, and
intravenous steroids (such as intravenous hydrocortisone or methylprednisolone), infliximab (IFX) and AZA, cyclosporin and AZA, IFX, adalimumab, golimumab and AZA, surgery and/or biological therapy (such as Natalizumab) if the determined severity is severe.

In another embodiment, the method comprises administering to a subject determined to have mild GI inflammation with a treatment selected from the group consisting of: topical mesalamine, oral mesalamine, topical prednisone, topical prednisolone, and/or budesonide.

In another embodiment, the method comprises administering to a subject determined to have moderate GI inflammation with a treatment selected from the group consisting of: systemic and topical steroids, oral mesalamine, ozanimod, upadacitinib, tofacitinib, risankizumab, ustekinumab, vedolizumab, azathioprine (AZA), thiopurines and/or budesonide. In another embodiment, the method comprises administering to a subject determined to have moderate GI inflammation with a treatment selected from the group consisting of: systemic and topical prednisone or prednisolone, oral mesalamine, ozanimod, upadacitinib, tofacitinib, risankizumab, ustekinumab, vedolizumab, azathioprine (AZA), thiopurines and/or budesonide.

In another embodiment, the method comprises administering to a subject determined to have severe GI inflammation with a treatment selected from the group consisting of: intravenous steroids, intravenous methylprednisolone, infliximab (IFX) and AZA, cyclosporin and AZA, IFX, adalimumab, golimumab and AZA, surgery and/or natalizumab. In another embodiment, the method comprises administering to a subject determined to have severe GI inflammation with a treatment selected from the group consisting of: intravenous hydrocortisone, intravenous methylprednisolone, infliximab (IFX) and AZA, cyclosporin and AZA, IFX, adalimumab, golimumab and AZA, surgery and/or natalizumab.

In another aspect, the invention provides a method of identifying GI inflammation, comprising:
i. providing a fecal RNA sample of a subject suspected of having GI inflammation,
ii. determining the levels in the sample of a plurality of human gene products, thereby obtaining the transcriptomic signature of the sample with respect to the plurality of gene products, and
iii. comparing the transcriptomic signature of said sample to a control transcriptomic signature by a supervised classification algorithm,
   wherein said plurality of gene products comprises: Group E gene products; Group F gene products; Group E2 gene products; Group F1 gene products; Group F2 gene products; and/or Group G gene products,
   wherein the outcome of the comparison is indicative of the presence or absence of GI inflammation in said subject, and wherein the method further comprises providing said subject with a suitable treatment selected from the group consisting of:
corticosteroids, prednisone, prednisolone, methylprednisolone, 5-ASA drugs, olsalazine, sulfasalazine, immunomodulators, azathioprine, mercaptopurine, TNF-alpha inhibitors, certolizumab; natalizumab; ustekinumab, vedolizumab and surgery.

### Gene Products

Gene products in accordance with embodiments of the invention include transcription products of various human genes as described herein. In various embodiments, the gene products may be assayed as mRNA transcripts directly from the fecal RNA sample (in particular, an rRNA-depleted sample), or as reverse-transcribed (cDNA) versions thereof. In some embodiments, the level of one or more gene products as disclosed herein is assayed in a library of gene products produced by reverse transcription of rRNA-depleted fecal RNA samples as described herein. In some embodiments, the levels of a plurality of human gene products selected from Table 1 below is determined in the fecal RNA sample.

Table 1 below lists exemplary gene products to be assayed in embodiments of the invention.

TABLE 1
Exemplary gene products

| Gene | Gene product accession number |
|---|---|
| ACSL1 | NM_001286708, NM_001286710, |
| | NM_001286711, NM_001381877, |
| | NM_001381878, NM_001381879, |
| | NM_001381880, NM_001381881, |
| | NM_001381882, NM_001381883, |
| | NM_001381884, NM_001381885, |
| | NM_001381886, NM_001381887, |
| | NM_001381888, NM_001381889, |
| | NM_001381890, NM_001995 |
| ADGRG3 | NM_001308360, NM_170776 |
| ALDOB | NM_000035 |
| ALOX5AP | NM_001204406, NM_001629 |
| ALPL | NM_000478, NM_001127501, |
| | NM_001177520, NM_001369803, |
| | NM_001369804, NM_001369805 |
| AMN | NM_030943 |
| ANP32E | NM_001136478, NM_001136479, NM_001280559, |
| | NM_001280560, NM_030920 |
| AOC1 | NM_001091, NM_001272072 |
| APBB1IP | NM_019043 |
| APOA1 | NM_000039, NM_001318017, |
| | NM_001318018, NM_001318021 |
| APOA4 | NM_000482 |
| APOB | NM_000384 |
| APOC3 | NM_000040 |
| AQP9 | NM_001320635, NM_001320636, NM_020980 |
| ARHGAP26 | NM_001135608, NM_001349547, NM_015071 |
| ARHGAP30 | NM_001025598, NM_001287600, |
| | NM_001287602, NM_181720 |
| ARRB2 | NM_001257328, NM_001257329, |
| | NM_001257330, NM_001257331, |
| | NM_001330064, NM_004313, NM_199004 |
| BCL6 | NM_001130845, NM_001134738, NM_001706 |
| BID | NM_001196, NM_001244567, |
| | NM_001244569, NM_001244570, |
| | NM_001244572, NM_197966, NM_197967 |
| CAPZA1 | NM_006135 |
| CASP5 | NM_001136109, NM_001136110, |
| | NM_001136112, NM_004347 |
| CCL3 | NM_002983 |
| CCL3L3 | NM_001001437, NM_021006 |
| CCL4 | NM_002984 |
| CCL4L2 | NM_001291468, NM_001291469, |
| | NM_001291470, NM_001291471, |
| | NM_001291472, NM_001291473, NM_001291474, |
| | NM_001291475, NM_207007 |
| CCR1 | NM_001295 |
| CD44 | NM_000610, NM_001001389, |
| | NM_001001390, NM_001001391, |
| | NM_001001392, NM_001202555, |
| | NM_001202556, NM_001202557 |
| CD53 | NM_000560, NM_001040033, NM_001320638 |
| CD83 | NM_001040280, NM_001251901, NM_004233 |
| CDHR2 | NM_001171976, NM_017675 |
| CDHR5 | NM_001171968, NM_021924, NM_031264 |
| CLEC2B | NM_005127 |
| CLEC4E | NM_001410969, NM_014358 |
| CLEC7A | NM_022570, NM_197947, NM_197948, |
| | NM_197949, NM_197950, NM_197954 |
| CMTM2 | NM_001199317, NM_144673 |
| CREB3L3 | NM_001271995, NM_001271996, |
| | NM_001271997, NM_032607 |
| CREM | NM_001267562, NM_001267563, |
| | NM_001267564, NM_001267565, |
| | NM_001267567, NM_001267570, |
| | NM_001352445, NM_001352446, |
| | NM_001352465, NM_001352466, |
| | NM_001352467, NM_001394595, |
| | NM_001394598, NM_001394600, |
| | NM_001394602, NM_001394603, |
| | NM_001394605, NM_001394608, |
| | NM_001394610, NM_001394613, |
| | NM_001394614, NM_001394615, |
| | NM_001394616, NM_001394617, |
| | NM_001394618, NM_001394619, |
| | NM_001394620, NM_001394622, |
| | NM_001394623, NM_001394625, |
| | NM_001394626, NM_001394627, |
| | NM_001394628, NM_001394629, |
| | NM_001394630, NM_001394631, |
| | NM_001881, NM_181571, NM_182717, |
| | NM_182718, NM_182719, NM_182720, |
| | NM_182721, NM_182723, NM_182724, |
| | NM_182769, NM_182770, NM_182771, |
| | NM_182772, NM_183011, NM_183012, |
| | NM_183013, NM_183060 |
| CSF3R | NM_000760, NM_156039, NM_172313 |
| CXCL8 | NM_000584, NM_001354840 |
| CXCR2 | NM_001168298, NM_001557 |
| CYP3A4 | NM_001202855, NM_017460 |
| CYTH1 | NM_001292018, NM_001292019, |
| | NM_001365037, NM_001365038, |
| | NM_001365039, NM_001365040, |
| | NM_001365041, NM_001394676, |
| | NM_001394677, NM_001394678, |
| | NM_004762, NM_017456 |
| CYTH4 | NM_001318024, NM_013385 |
| DDX21 | NM_001256910, NM_001410932, NM_004728 |
| DEFA1B | NM_001042500, NM_001302265 |
| DEFA3 | NM_005217 |
| DEFA5 | NM_021010 |
| DEFA6 | NM_001926 |
| DNAJA1 | NM_001314039, NM_001539 |
| DPEP1 | NM_001128141, NM_001389466, |
| | NM_001389467, NM_001389468, |
| | NM_001389469, NM_001389470, |
| | NM_001389471, NM_004413 |
| EIF3J | NM_001284335, NM_001284336, NM_003758 |
| EPS8L1 | NM_017729, NM_133180 |
| ERICH1 | NM_001303100, NM_207332 |
| EWSR1 | NM_001163285, NM_001163286, |
| | NM_001163287, NM_005243, NM_013986 |
| FABP2 | NM_000134 |
| FABP6 | NM_001040442, NM_001130958, NM_001445 |
| FAM129A | NM_052966 |
| (NIBAN1) | |
| FCER1G | NM_004106 |
| FCGR2A | NM_001136219, NM_001375296, |
| | NM_001375297, NM_021642 |
| FCGR3A | NM_000569, NM_001127592, |
| | NM_001127593, NM_001127595, |
| | NM_001127596, NM_001329120, |
| | NM_001329122, NM_001386450 |
| FCGR3B | NM_000570, NM_001244753, |
| | NM_001271035, NM_001271036 |
| FNBP1 | NM_001363755, NM_001411018, NM_015033 |
| FPR1 | NM_001193306, NM_002029 |
| FYB1 | NM_001243093, NM_001349333, NM_001465, |
| | NM_018594, NM_199335 |
| GOS2 | NM_015714 |
| GBP1 | NM_002053 |
| GBP4 | NM_052941 |
| GBP5 | NM_001134486, NM_001391920, NM_052942 |
| GDF5OS | NR_161326 |
| (GDF5- | |
| AS1) | |
| GMFG | NM_001301008, NM_001411106, NM_004877 |
| GNA11 | NM_002067 |
| GPR65 | NM_003608 |
| GUCA2A | NM_033553 |
| GUCA2B | NM_007102 |
| HCAR2 | NM_177551 |
| HCAR3 | NM_006018 |
| HNRNPA2 | NM_002137, NM_031243 |
| B1 | |
| ICAM1 | NM_000201 |
| IFI16 | NM_001206567, NM_001364867, |
| | NM_001376587, NM_001376588, |
| | NM_001376589, NM_001376591, |
| | NM_001376592, NM_005531 |
| IFIT2 | NM_001547 |
| IFIT3 | NM_001031683, NM_001289758, |
| | NM_001289759, NM_001549 |
| IFITM2 | NM_006435 |
| IGSF6 | NM_005849 |
| IL1B | NM_000576 |
| IL1R2 | NM_001261419, NM_004633 |
| IL1RAP | NM_001167928, NM_001167929, |
| | NM_001167930, NM_001167931, |
| | NM_001364879, NM_001364880, |
| | NM_001364881, NM_002182, NM_134470 |
| IL1RN | NM_000577, NM_001318914, |
| | NM_001379360, NM_173841, NM_173842, |
| | NM_173843 |
| ILF3 | NM_001137673, NM_001394808, |
| | NM_001394809, NM_001394810, |
| | NM_001394811, NM_001394812, |
| | NM_001394813, NM_001394814, |
| | NM_001394815, NM_001394816, |
| | NM_001394817, NM_001394818, |
| | NM_001394819, NM_001394820, |
| | NM_001394821, NM_001394822, |
| | NM_001394823, NM_001394824, |
| | NM_001394826, NM_001394827, |
| | NM_004516, NM_012218, NM_017620, NM_153464 |
| ITGAX | NM_000887, NM_001286375 |
| ITSN2 | NM_001348181, NM_001348182, |
| | NM_001348183, NM_001348184, |
| | NM_001348185, NM_001348186, |
| | NM_006277, NM_019595, NM_147152 |
| KCNJ15 | NM_001276435, NM_001276436, |
| | NM_001276437, NM_001276438, |
| | NM_001276439, NM_002243, |
| | NM_170736, NM_170737 |
| KCNK6 | NM_004823 |
| KIAA0825 | NM_001145678, NM_001385712, |
| | NM_001385713, NM_001385714, |
| | NM_001385715, NM_001385716, |
| | NM_001385717, NM_001385719, |
| | NM_001385720, NM_001385721, |
| | NM_001385722, NM_001385723, |
| | NM_001385724, NM_001385728, |
| | NM_001385729, NM_001385730, |
| | NM_001388325, NM_173665 |
| KIAA1109 | NM_001384125, NM_015312 |
| (BLTP1) | |
| LCP1 | NM_002298 |
| LCP2 | NM_005565 |
| LILRB3 | NM_001081450, NM_001320960, NM_006864 |
| LRRK2 | NM_198578 |
| LSP1 | NM_001013253, NM_001013254, |
| | NM_001013255, NM_001242932, |
| | NM_001289005, NM_002339 |
| LYN | NM_001111097, NM_002350 |
| MAVS | NM_001206491, NM_001385663, NM_020746 |
| MEP1A | NM_005588 |
| METAP2 | NM_001317182, NM_001317183, |
| | NM_001330246, NM_006838 |
| MNDA | NM_002432 |
| MTRNR2L1 | NM_001190452 |
| MTTP | NM_000253, NM_001300785, NM_001386140 |
| NAMPT | NM_005746 |
| NBN | NM_001024688, NM_002485 |
| NCF2 | NM_000433, NM_001127651, NM_001190789, |
| | NM_001190794, NM_001410895 |
| NCL | NM_005381 |
| OLR1 | NM_001172632, NM_001172633, NM_002543 |
| OSM | NM_001319108, NM_020530 |
| PDE4B | NM_001037339, NM_001037340, |
| | NM_001037341, NM_001297440, |
| | NM_001297441, NM_001297442, NM_002600 |
| PFKFB3 | NM_001145443, NM_001282630, |
| | NM_001314063, NM_001323016, |
| | NM_001323017, NM_001363545, NM_004566 |
| PHACTR1 | NM_001242648, NM_001322308, |
| | NM_001322309, NM_001322310, |
| | NM_001322311, NM_001322312, |
| | NM_001322313, NM_001322314, |
| | NM_001374581, NM_001374583, |
| | NM_001374584, NM_030948 |
| PHIP | NM_017934 |
| PLAU | NM_001145031, NM_001319191, NM_002658 |
| PLCB2 | NM_001284297, NM_001284298, |
| | NM_001284299, NM_004573 |
| PLCD3 | NM_133373 |
| PLEK | NM_002664 |
| PPIF | NM_005729 |
| PROK2 | NM_001126128, NM_021935 |
| PTGS2 | NM_000963 |
| RAPGEF6 | NM_001164386, NM_001164387, |
| | NM_001164388, NM_001164389, |
| | NM_001164390, NM_016340 |
| RCC1 | NM_001048194, NM_001048195, |
| | NM_001048199, NM_001269, |
| | NM_001381865, NM_001381866 |
| REGIA | NM_002909 |
| REG1B | NM_006507 |
| RHOH | NM_001278359, NM_001278360, |
| | NM_001278361, NM_001278362, |
| | NM_001278363, NM_001278364, |
| | NM_001278365, NM_001278366, |
| | NM_001278367, NM_001278368, |
| | NM_001278369, NM_004310 |
| S100A4 | NM_002961, NM_019554 |
| S100A9 | NM_002965 |
| SAMSN1 | NM_001256370, NM_001256579, |
| | NM_001286523, NM_001395857, |
| | NM_001395858, NM_022136 |
| SELENOP | NM_001085486, NM_001093726, NM_005410 |
| SELL | NM_000655 |
| SH3BP5 | NM_001018009, NM_004844 |
| SI | NM_001041 |
| SIPA1L2 | NM_001377488, NM_020808 |
| SLC15A1 | NM_005073 |
| SLC2A3 | NM_006931 |
| SLC5A1 | NM_000343, NM_001256314 |
| SMIM24 | NM_001136503 |
| SNX10 | NM_001199835, NM_001199837, |
| | NM_001199838, NM_001318198, |
| | NM_001318199, NM_001362753, |
| | NM_001362754, NM_013322 |
| SOCS3 | NM_001378932, NM_001378933, NM_003955 |
| SOD2 | NM_000636, NM_001024465, |
| | NM_001024466, NM_001322814, |
| | NM_001322815, NM_001322816, |
| | NM_001322817, NM_001322819, |
| | NM_001322820 |
| SP140 | NM_001005176, NM_001278451, NM_001278452, |
| | NM_001278453, NM_007237 |
| SRGN | NM_001321053, NM_001321054, NM_002727 |
| SUPT6H | NM_001320755, NM_003170 |
| SYNE2 | NM_015180, NM_182910, NM_182913, NM_182914 |
| TANK | NM_001199135, NM_004180, NM_133484 |
| TET3 | NM_001287491, NM_001366022 |
| TLR2 | NM_001318787, NM_001318789, |
| | NM_001318790, NM_001318791, |
| | NM_001318793, NM_001318795, |
| | NM_001318796, NM_003264 |
| TM4SF5 | NM_003963 |
| TMEM154 | NM_152680 |
| TNFAIP6 | NM_007115 |
| TRA2B | NM_001243879, NM_004593 |
| TREM1 | NM_001242589, NM_001242590, NM_018643 |
| UTRN | NM_001375323, NM_007124 |
| VNN2 | NM_001242350, NM_004665, NM_078488 |
| WDR66 | NM_001178003, NM_144668 |
| (CFAP251) | |
| YTHDC1 | NM_001031732, NM_001330698, NM_133370 |
| ZEB2 | NM_001171653, NM_014795 |
| ZFC3H1 | NM_144982 |
| ZNF267 | NM_001265588, NM_003414 |
| ZNF511- | NM_001396060 |
| PRAP1 | |

Table 2 below presents groups of gene products that may be used in various diagnostic classifiers as described and exemplified herein.

TABLE 2
Gene product groups

| Group/Description | Genes |
|---|---|
| A | TET3, RAPGEF6, RCC1, IL1R2, |
| Elevated in | WDR66, ANP32E, EPS8L1, ALPL, |
| colonic | ILF3, ERICH1, PLCD3, MAVS, |
| inflammation | SIPA1L2, ARHGAP30, FNBP1, NCL, |
| that does | EWSR1, SP140, DDX21, KCNK6, |
| not involve the | KIAA0825, SUPT6H, CYTH1, |
| terminal ileum | EIF3J, ARHGAP26, MTRNR2L1, |
| | PLCB2, UTRN, METAP2, GDF5OS |
| B | REGIA, FABP6, REGIB, APOB, |
| Elevated in | ALDOB, ZNF511-PRAP1, SI, |
| terminal | CYP3A4, APOA1, DEFA6, |
| ileal | APOA4, DPEP1, DEFA5, APOC3, |
| inflammation | CREB3L3, AMN, SLC15A1, |
| | GUCA2A, SMIM24, MTTP, SLC5A1, |
| | FABP2, MEPIA, TM4SF5, |
| | GUCA2B, AOC1, CDHR5, CDHR2, |
| | SELENOP, GNA11 |
| C | AC007192.1, ACSL1, ALOX5AP, |
| Elevated | AQP9, BCL6, CCL4, CD44, CSF3R, |
| in moderate | CXCL8, DEFA1B, FAM129A, |
| to severe GI | FCGR2A, FCGR3B, FPR1, FYB1, |
| inflammation | GOS2, GBP1, GMFG, HCAR2, |
| | HCAR3, ICAM1, IFI16, IFITM2, IL1B, |
| | ILIRN, ITGAX, LCP1, LCP2, |
| | LILRB3, LYN, MNDA, NAMPT, NCF2, |
| | OSM, PDE4B, PFKFB3, PLEK, |
| | PPIF, PROK2, PTGS2, S100A4, |
| | S100A9, SAMSN1, SLC2A3, |
| | SOCS3, SOD2, SRGN, TNFAIP6, |
| | TREM1, ZNF267 |
| D | CAPZA1, CASP5, DNAJA1, |
| Elevated in | HNRNPA2B1, ITSN2, KIAA1109, PHIP, |
| undetectable | SYNE2, TANK, TRA2B, YTHDC1, ZFC3H1 |
| to low GI | |
| inflammation | |
| E | GBP5, DEFA3, DEFA1B, FCGR3A, |
| Elevated in | CLEC2B, SH3BP5, CD53, |
| active GI | CLEC7A, PLAU, IL1RAP, CCR1, |
| inflammation | FCERIG, CLEC4E, TLR2, LSP1, |
| | ADGRG3, CD83, TREM1, CMTM2, |
| | RHOH, APBBIIP, LRRK2, BID, |
| | KCNJ15, IFIT2, PLEK, PDE4B, |
| | HCAR3, ITGAX, SELL, MNDA, |
| | HCAR2, ICAM1, FCGR2A, |
| | CCL4L2, CCL3L3, GBP1, CSF3R, CD44, |
| | PROK2, SOCS3, GMFG, S100A4, |
| | TNFAIP6, SNX10, NBN, OSM, |
| | SOD2, IFI16, FYB1, AC007192.1, |
| | FCGR3B, IL1B, CYTH4, ILIRN, |
| | OLR1, VNN2, CCL3, CCL4, IFIT3, |
| | CREM, ZEB2, ALOX5AP, |
| | CXCL8, LCP2, IGSF6, CXCR2, |
| | ZNF267, GBP4, LCP1, PHACTR1, |
| | ARRB2, TMEM154, BCL6, GPR65 |
| F | GBP5, DEFA3, DEFA1B, FCGR3A, |
| Elevated | CLEC2B, SH3BP5, CD53, |
| in active GI | CLEC7A, PLAU, IL1RAP, CCR1, |
| inflammation | FCERIG, CLEC4E, TLR2, LSP1, |
| | ADGRG3, CD83, TREM1, CMTM2, |
| | RHOH, APBBIIP, LRRK2, BID, |
| | KCNJ15, IFIT2, PLEK, PDE4B, |
| | HCAR3, ITGAX, SELL, MNDA, |
| | HCAR2, ICAM1, FCGR2A, CCL4L2 |

In some embodiments, the plurality of gene products to be assayed in accordance with embodiments of the invention includes a subset of gene products as identified in one or more of Groups A-F (or, in other embodiments, in Tables 1 or 4). Table 3 below presents groups of gene products that are particularly advantageous in embodiments of the invention when examining solid fecal (stool) samples. In particular, gene products in the exemplary groups identified in Table 3 below may be used in classifiers for identifying active GI inflammation (such as IBD flares) using stool samples, as described and exemplified herein.

TABLE 3
Additional gene signatures for detecting active inflammation

| Group | Genes |
|---|---|
| G | RNASEK, RNASEK-C17orf49, |
| Elevated in | MIDN, HLA-A, B2M, HLA-B, HLA- |
| active GI | C, CAP1, PFN1, ACTB, MXD1, |
| inflammation | SAT1, LITAF, NFKBIA, S100A9, |
| | S100A8, EIF1, FTL, FTH1 |
| H | CALM2, CCL4, CDKNIA, |
| Elevated in | CEACAM1, CEBPB, CXCL8, EGR1, |
| active GI | AC138811.2, AQP9, ARPC2, ARPC5, |
| inflammation | BASP1, BCL2A1, BRI3, BTG2, |
| | ETS2, FOS, FPR1, FTL, GOS2, |
| | GABARAP, GLUL, GNB2, HCAR3, |
| | HLA-C, HLA-E, ICAM1, IFITM1, |
| | IFITM2, IL1B, IL1RN, IRF1, |
| | ISG20, ITM2B, IVNS1ABP, |
| | KDM6B, KLF6, LITAF, MARCKS, |
| | MCL1, MXD1, NAMPT, NFKBIA, |
| | OSM, PFN1, PLAUR, PLEK, |
| | PNRC1, PPIF, PROK2, PTP4A1, |
| | S100A11, S100A8, S100A9, SAT1, |
| | SDCBP, SLC25A37, SOCS3, SOD2, |
| | SRGN, TAF10, TNFAIP3, TPM4, |
| | TXNIP, TYMP, UBE2B, VASP, |
| | WDR83OS, ZFP36, ZFP36L1 |
| I | CCL4, CXCL8, HCAR3, ICAM1, |
| Elevated in active | IL1B, ILIRN, OSM, PLEK, PROK2, |
| GI inflammation | SOCS3, SOD2 |
| J | ABHD17C, AC005943.1, ACTN4, |
| Downregulated in | ALDOA, AP000350.4, AP000721.1, |
| active GI | AP003419.1, C17orf49, CA4, CDHR5, |
| inflammation | CFL1, CKB, COX7A2, COX8A, |
| | CRIP1, CST3, CTNND1, DBNDD2, |
| | DYNLRB1, EGLN1, EIF4G2, |
| | EPCAM, FABP1, FCGBP, FXYD3, |
| | GUCA2A, GUCA2B, IFI27, |
| | KRT8, LGALS4, LYPD8, MAL2, |
| | MGLL, MIF, MISP, MUC12, MUC2, |
| | MYL12B, OST4, P2RX5-TAX1BP3, |
| | PDLIM1, PHGR1, PIGR, PLAC8, |
| | POLD4, PPDPF, RHOC, S100A16, |
| | SDCBP2, SERINC2, SFN, |
| | SH3BGRL3, SMIM22, SPINT2, |
| | SRI, STK24, SYS1-DBNDD2, |
| | TAX1BP3, TFF1, TFF3, TMEM54, |
| | TMPRSS2, TMSB10, TRIM31, |
| | UBA52, UBB, UQCR11, ZG16 |

In some embodiments the plurality of gene products further comprises at least one additional gene product selected from Table 4 below. In yet other embodiments, the plurality of gene products to be determined is selected from Table 4.

TABLE 4
Additional gene products
Additional genes for gene product determination

AAK1, ABCA8, ABCB1, ABCF1, ABCG1, ABCG2, ABHD3, AC003688.1, AC004922.1,
AC005726.2, AC005943.1, AC007192.1, AC008695.1, AC008763.3, AC008764.1,
AC008878.3, AC009779.3, AC011479.1, AC013394.1, AC024592.3, AC036214.3,
AC046185.1, AC068580.4, AC073610.3, AC078927.1, AC093525.2, AC099329.3,
AC107871.1, AC135178.2, AC138811.2, AC245033.1, ACAA2, ACADVL, ACAP2, ACBD3,
ACE2, ACIN1, ACOX1, ACSL5, ACSS2, ACTB, ACTG1, ACTN1, ACTN4, ACTR2, ACTR3,
ADAM10, ADAR, ADIPOR2, ADIRF, AF241726.2, AFDN, AFF4, AFTPH, AGAP1,
AGPAT2, AGR2, AGR3, AHCY, AHNAK, AK1, AKAP13, AKAP17A, AKAP9, AL713999.1,
ALDH2, ALDOA, ALDOB, ALPL, AMN, ANKHD1, ANKLE2, ANKRD11, ANKRD17,
ANKRD36, ANKRD36B, ANKRD36C, ANP32A, ANP32B, ANP32E, ANPEP, ANXA11,
ANXA2, AOC1, AP000311.1, AP000350.4, AP3B1, AP3D1, APC, APLP2, APOA1, APOA4,
APOB, APOBR, APOC3, APOL1, APOL2, APOL6, APP, APPL2, AQP8, ARAP1, ARAP2,
ARF1, ARF6, ARFGEF1, ARGLU1, ARHGAP21, ARHGAP26, ARHGAP30, ARHGAP35,
ARHGAP5, ARHGDIA, ARHGEF1, ARHGEF12, ARHGEF35, ARID1B, ARID3A, ARID4A,
ARID4B, ARMC9, ARPC2, ARPC4, ARPC5, ASH1L, ASL, ASPH, ASS1, ATF4, ATG12,
ATP10B, ATP1A1, ATP1B1, ATP1B3, ATP2A2, ATP2A3, ATP2B1, ATP5B, ATP5E,
ATP5G3, ATP5I, ATP5J, ATP5L, ATP6V0A1, ATP6V0B, ATP6V0E1, ATP8B1, ATPIF1,
ATRX, B2M, B3GALT5, BAG1, BAIAP2L1, BAZ1A, BAZ1B, BAZ2A, BAZ2B, BCAP31,
BCAS1, BCL2A1, BCL2L14, BCL2L2PABPN, BCLAF1, BDP1, BECN1, BIRC3, BIRC6,
BIVMERCC5, BOD1L1, BPTF, BRD2, BRD4, BTF3, BTN3A2, C11orf58, C14orf2, C14orf37,
C15orf48, C2orf88, C4orf3, C4orf48, C6orf62, C9orf78, CA1, CA12, CA2, CA4, CAB39,
CACNA1A, CALCOCO2, CALM1, CALM3, CALR, CAMK2N1, CANX, CAP1, CAPN2,
CAPN8, CAPNS1, CAPZA2, CAPZB, CASC3, CASC4, CASP4, CASP5, CASP7, CAST,
CBFB, CBX3, CCDC186, CCDC68, CCDC88B, CCDC92, CCNI, CCNL1, CCPG1, CD164,
CD24, CD2AP, CD44, CD46, CD58, CD63, CD74, CD82, CD9, CDC42, CDC42BPA,
CDC42SE2, CDC5L, CDH1, CDH17, CDHR2, CDHR5, CDK11A, CDK11B, CDK19,
CDKN1A, CDV3, CDX2, CEACAM1, CEACAM5, CEACAM6, CEACAM7, CEBPZOS,
CEP350, CES2, CFDP1, CFL1, CFLAR, CGN, CHCHD2, CHD1, CHD2, CHD3, CHD4, CHD9,
CHMP2A, CHMP2B, CHMP3, CHMP4B, CHP1, CHURC1, CIB1, CIR1, CIRBP, CISD3,
CKAP4, CKB, CLCA1, CLCA4, CLDN3, CLDN4, CLDN7, CLIC5, CLINT1, CLIP1, CLK1,
CLMN, CLSTN1, CLTB, CLTC, CMAS, CMBL, CMIP, CMPK1, CMTM6, CNBP, COL17A1,
COMMD6, CORO2A, COX4I1, COX5A, COX5B, COX6A1, COX6B1, COX6C, COX7A2,
COX7B, COX7C, COX8A, CPEB4, CREB3L3, CREBBP, CREBRF, CRIP1, CSDE1,
CSNK1A1, CST3, CSTB, CTAGE5, CTBP2, CTNNA1, CTNNB1, CTNND1, CTSA, CTSB,
CTSC, CTSS, CTTN, CTTNBP2NL, CWC25, CXADR, CXCL14, CYBA, CYCS, CYP3A4,
CYP3A5, CYSTM1, CYTH1, DAAM1, DAZAP2, DBI, DDX17, DDX18, DDX19B, DDX21,
DDX24, DDX27, DDX3X, DDX46, DDX5, DDX52, DDX6, DDX60, DEFA5, DEFA6, DEK,
DENND4A, DGAT1, DHFR, DHRS11, DHRS3, DHX36, DIAPH1, DIAPH2, DICER1, DIP2A,
DMXL2, DNAJA1, DNAJB6, DNAJC3, DNAJC8, DNASE1, DNM2, DNMBP, DNTTIP2,
DOCK5, DOCK7, DPEP1, DSC2, DSG2, DSP, DST, DSTN, DUOX2, DUOXA2, DYNC1H1,
DYNC1I2, DYNC1LI2, DYNLL1, DYNLRB1, DYNLT1, DYRK2, EDF1, EEA1, EEF1A1,
EEF1D, EEF1G, EEF2, EFCAB14, EFHD2, EFNA1, EGLN3, EHBP1L1, EI24, EIF1,
EIF2AK2, EIF2S2, EIF3A, EIF3J, EIF4A2, EIF4E2, EIF4EBP2, EIF4G1, EIF4G2, EIF4G3,
EIF5, EIF5B, ELF1, ELF3, ELMSAN1, ELOB, EMLA, EMP1, ENO1, EP300, EPB41L2,
EPCAM, EPN1, EPS8, EPS8L1, EPS8L2, EPS8L3, EPSTI1, ERAP2, ERBB3, ERBIN, ERC1,
ERICH1, ETFB, ETHE1, ETS2, ETV6, EVI5, EWSR1, EZR, F11R, FABP1, FABP2, FABP6,
FAM107B, FAM114A1, FAM120A, FAM120AOS, FAM126B, FAM133B, FAM13A,
FAM177A1, FAM32A, FAM3D, FAM46A, FAM50A, FAT1, FAU, FBLIM1, FBXW5,
FBXW7, FCGBP, FCGRT, FGD4, FGD6, FGL2, FKBP15, FKBP5, FLNB, FMR1, FNBP1,
FNBP4, FNDC3B, FOS, FOSL2, FOXN3, FOXP1, FRMD1, FRYL, FSIP2, FTH1, FTL,
FUBP1, FUS, FXYD3, FYB1, GABARAP, GAK, GALE, GALNT1, GAPDH, GBP1, GBP2,
GBP3, GCC2, GCNT3, GDF5OS, GGNBP2, GGT6, GHITM, GIGYF2, GIMAP4, GK, GLRX,
GLS, GLTP, GLYR1, GMFB, GNA11, GNAI2, GNAQ, GNAS, GNB1, GNG12, GNG5,
GOLGA2, GOLGA3, GOLGA4, GOLGB1, GOLIM4, GOLM1, GON4L, GPA33, GPBP1,
GPBP1L1, GPM6A, GPR85, GPRC5A, GPS2, GPX4, GRAP2, GRB2, GREM1, GRM5, GRN,
GSN, GSR, GSTP1, GTF2F1, GTF2I, GUCA2A, GUCA2B, GUK1, H2AFJ, H2AFY, H3F3A,
H3F3B, HADHA, HBA2, HBB, HDAC1, HDGF, HDLBP, HEBP2, HECTD1, HEPH, HERC4,
HERPUD1, HES1, HHLA2, HIF1A, HINT1, HIST1H1C, HIST1H4C, HK2, HLAA, HLAB,
HLAC, HLADRA, HLADRB1, HLAE, HLAF, HM13, HMGA1, HMGB1, HMGB2, HMGN1,
HNRNPA2B1, HNRNPA3, HNRNPAB, HNRNPC, HNRNPDL, HNRNPF, HNRNPH1,
HNRNPK, HNRNPM, HNRNPU, HOOK3, HOXB7, HPGD, HS3ST1, HSBP1, HSP90AA1,
HSP90AB1, HSP90B1, HSPA1A, HSPA5, HSPA8, HSPD1, ID2, IER2, IFI16, IFI27, IFITM2,
IFITM3, IGF2BP2, IGF2R, IK, IL10RB, IL1R2, IL32, IL6R, IL6ST, ILF3, INF2, INHBA,
INTS6, IQGAP1, IQGAP2, IRF1, ISG15, IST1, ITGA6, ITM2B, ITM2C, ITPR2, ITSN1,
ITSN2, IVNS1ABP, IWS1, JAK1, JMJD1C, JPT1, JTB, JUN, JUND, JUP, KANSL1, KAT6A,
KAT6B, KCNJ3, KCNK6, KDELR1, KDM2A, KDM3A, KDM5A, KDM5B, KDM7A,
KHDC4, KIAA0232, KIAA0825, KIAA1109, KIAA1217, KIAA1551, KIAA2026,
KIDINS220, KIF13B, KIF16B, KIF1B, KIF1C, KIF3B, KIF5B, KLF3, KLF5, KLF6, KMT2A,
KMT2B, KMT2C, KMT2E, KPNA4, KPNB1, KRAS, KRCC1, KRT18, KRT19, KRT20,
KRT8, KTN1, LAMA3, LAMB3, LAMC2, LAMP1, LAMTOR4, LAMTOR5, LAP3, LARP7,
LASP1, LCMT2, LCN2, LCOR, LCP1, LDHA, LDLR, LGALS2, LGALS3, LGALS3BP,
LGALS4, LIMA1, LIPH, LITAF, LLGL2, LMNA, LMO7, LMTK2, LPGAT1, LPIN2, LPP,
LRP1, LRP10, LRRC69, LRRFIP1, LRRFIP2, LTB, LTBR, LUC7L3, LUZP1, LYPD8, LYZ,
MACF1, MAGI1, MAGI3, MALL, MAN1A2, MAP2K2, MAP2K3, MAP3K11, MAP3K13,
MAP3K2, MAP4K4, MAP7, MAP7D1, MAPK6, MARCKS, MARK3, MARVELD3, MATR3,
MAVS, MBD4, MBNL1, MBP, MCTP2, MDK, MDM4, MED12L, MED13, MED13L,
MEF2A, MEP1A, METAP2, MFAP1, MFSD2A, MGAM, MGEA5, MGLL, MIA3, MICAL3,
MIDN, MIER1, MIER3, MINOS1, MINOS1NBL1, MISP, MKNK2, MLKL, MME, MORF4L1,
MORF4L2, MPHOSPH8, MPP5, MPST, MS4A12, MSL1, MSN, MT1G, MT2A, MTDH,
MTPN, MTRNR2L1, MTRNR2L10, MTRNR2L3, MTRNR2L6, MTTP, MUC12, MUC13,
MUC17, MUC2, MUC3A, MUC4, MVP, MXD1, MXI1, MYCBP2, MYH14, MYH9,
MYL12A, MYL12B, MYL6, MYL6B, MYLK, MYO10, MYO15B, MYO18A, MYO1A,
MYO1E, MYO6, MYO9B, N4BP2L2, NABP1, NACA, NAPRT, NASP, NBEAL1, NBPF1,
NBPF14, NBPF19, NBR1, NCALD, NCL, NCOA4, NCOR1, NDRG1, NDUFA1, NDUFA13,
NDUFA2, NDUFA3, NDUFB1, NDUFB2, NDUFB7, NEDD4L, NEDD8MDP1, NEDD9,
NEMF, NET1, NEUROD2, NF1, NFE2L1, NFE2L2, NFKBIA, NIN, NIPBL, NKTR, NLRC5,
NMT1, NMT2, NOP56, NOSIP, NOSTRIN, NPEPPS, NPIPB13, NPIPB5, NRDC, NSD2,
NSD3, NSRP1, NT5C3A, NUB1, NUDT4, NUMB, NUPR1, OAZ1, OFD1, OPTN, OS9,
OSBPL2, OSBPL8, OST4, P2RX4, P4HB, PABPC1, PABPC4, PACSIN2, PAFAH1B1, PAG1,
PAK1, PAPOLA, PARD3, PARM1, PARP14, PARP9, PATJ, PBRM1, PBXIP1, PCBP1,
PCBP2, PCF11, PCK1, PCM1, PDCD10, PDCD4, PDCD6IP, PDE9A, PDGFA, PDLIM1,
PDLIM5, PDPK1, PDXDC1, PDXK, PECAM1, PERP, PEX26, PFDN5, PFN1, PGK1,
PHACTR2, PHACTR4, PHF20L1, PHF3, PHGR1, PHIP, PI3, PIGR, PIP5K1B, PJA2, PKIB,
PKM, PKN2, PLA2G2A, PLAC8, PLAUR, PLCB2, PLCD3, PLCE1, PLEC, PLEKHA7,
PLEKHB2, PLIN2, PLIN3, PLOD2, PLS1, PLSCR1, PMP22, PNISR, PNN, PNRC1, PNRC2,
POLD4, POLR2L, POMP, POU2F2, PPARA, PPDPF, PPFIA1, PPIG, PPM1G, PPP1CA,
PPP1CB, PPP1R12A, PPP2R5C, PPP4R2, PRDM1, PRDM2, PRDX5, PRDX6, PRELID1,
PRKCB, PRKDC, PRMT2, PRPF38B, PRPF40A, PRPF4B, PRR13, PRR15, PRR15L, PRR5L,
PRRC2C, PRSS3, PRSS8, PSAP, PSEN1, PSMA4, PSMA7, PSMB1, PSMB3, PSMB8, PSMB9,
PSMC1, PSMD1, PSMD7, PSME1, PSME2, PTBP3, PTMA, PTMS, PTPN12, PTPRC, PTPRF,
PTPRH, PTTG1IP, RAB10, RAB11A, RAB11FIP1, RAB2A, RAB8A, RABAC1, RABEP1,
RABL6, RAC1, RACK1, RAD21, RALB, RALBP1, RALGAPA2, RALGPS2, RALY,
RANBP2, RAP1B, RAPGEF6, RARRES3, RASEF, RASSF6, RB1CC1, RBBP6, RBM25,
RBM3, RBM33, RBM39, RBM47, RBM6, RBM8A, RBP2, RBPJ, RC3H1, RCAN1, RCC1,
REG1A, REG1B, REG4, REL, RETREG1, RFK, RHOA, RHOC, RIF1, RIOK3, RNASE1,
RNASEK, RND3, RNF145, RNF149, RNF19A, RNF213, RNMT, RNPC3, ROCK1, ROCK2,
ROMO1, RPGR, RPL10, RPL11, RPL12, RPL13, RPL13A, RPL14, RPL15, RPL17C18orf32,
RPL18, RPL18A, RPL19, RPL21, RPL22, RPL23, RPL23A, RPL24, RPL27, RPL27A, RPL28,
RPL29, RPL3, RPL30, RPL31, RPL32, RPL34, RPL35, RPL35A, RPL36, RPL36A, RPL36AL,
RPL37, RPL37A, RPL38, RPL39, RPL4, RPL41, RPL5, RPL6, RPL7, RPL7A, RPL8, RPL9,
RPLP0, RPLP1, RPLP2, RPN1, RPN2, RPS10NUDT3, RPS11, RPS12, RPS13, RPS14, RPS15,
RPS15A, RPS16, RPS17, RPS18, RPS19, RPS2, RPS20, RPS21, RPS23, RPS24, RPS25,
RPS26, RPS27, RPS27A, RPS27L, RPS28, RPS29, RPS3, RPS3A, RPS4X, RPS5, RPS6,
RPS6KA5, RPS7, RPS8, RPS9, RPSA, RRBP1, RREB1, RSBN1, RSBN1L, RSF1, RSRC2,
RSRP1, RTF1, RTFDC1, RTN4, RXRA, S100A10, S100A11, S100A14, S100A16, S100A6,
SAFB, SAFB2, SAMD9, SAMD9L, SAMHD1, SAP30BP, SARAF, SAT1, SBDS, SBNO1,
SCAF11, SCAND1, SCARB2, SCIN, SCNN1A, SCO2, SCP2, SDCBP, SDCBP2, SEC61G,
SEC62, SECTM1, SELENBP1, SELENOK, SELENOP, SELENOS, SELENOW, SEM1,
SEMA3B, SEMA6A, SENP3EIF4A1, SENP5, SENP6, SEPHS2, SEPT7, SERBP1, SERF2,
SERINC2, SERINC3, SERP1, SERPINA1, SERPINB1, SERPINB6, SET, SETD2, SETD5,
SETX, SF3B1, SF3B2, SFN, SFPQ, SGK1, SH3BGRL3, SH3BP2, SH3D19, SH3GLB1,
SH3KBP1, SHOC2, SI, SIK3, SIPA1L2, SKAP2, SKIL, SKP1, SLC15A1, SLC16A3,
SLC17A4, SLC20A1, SLC22A18, SLC25A3, SLC25A37, SLC25A5, SLC25A6, SLC26A2,
SLC26A3, SLC27A4, SLC35A3, SLC39A7, SLC40A1, SLC43A2, SLC44A1, SLC44A4,
SLC4A4, SLC5A1, SLC5A12, SLC6A19, SLC6A8, SLC9A3R1, SLK, SLMAP, SLTM, SLU7,
SMAP2, SMARCA2, SMARCA4, SMARCA5, SMARCC1, SMC1A, SMC3, SMC4, SMC5,
SMCHD1, SMG1, SMIM14, SMIM22, SMIM24, SMIM31, SNRPD2, SNW1, SNX6, SNX9,
SOD1, SOD2, SON, SORL1, SOS2, SOX4, SP100, SP110, SP140, SP140L, SPAG9, SPATS2L,
SPECC1, SPECC1L, SPEN, SPG7, SPINK1, SPINK4, SPINK5, SPINT1, SPINT2, SPPL2A,
SPTAN1, SPTBN1, SQSTM1, SREK1, SREK1IP1, SRI, SRP14, SRPK1, SRPK2, SRRM1,
SRRM2, SRSF11, SRSF3, SRSF4, SRSF5, SSFA2, SSR4, SSU72, ST13, ST14, STAG1,
STAG2, STAP2, STAT1, STAT3, STAT6, STAU1, STAU2, STK17A, STK17B, STK24,
STK38, STK39, STK4, STPG4, SUB1, SUDS3, SULF2, SULT1A1, SUPT5H, SUPT6H, SVIL,
SYAP1, SYF2, SYK, SYNE1, SYNE2, SYNRG, SYTL2, SYTL4, TACC1, TAF1D, TAF7,
TAGLN2, TAOK1, TAOK3, TAP1, TAPBP, TAX1BP1, TAX1BP3, TBCA, TBL1XR1, TCF25,
TCF7L2, TCIRG1, TDP2, TES, TET2, TET3, TFF1, TFF3, TGOLN2, THOC2, THRAP3,
TIMP1, TJP1, TKT, TLN1, TM4SF5, TM9SF3, TMA7, TMBIM6, TMC5, TMCC3, TMCO1,
TMEM120A, TMEM131, TMEM258, TMEM37, TMEM45B, TMEM54, TMEM59,
TMEM87A, TMF1, TMIGD1, TMOD3, TMPRSS2, TMSB10, TMSB4X, TMX2CTNND1,
TNFAIP2, TNFRSF14, TNFRSF1A, TNFSF10, TNIP1, TNIP3, TNRC6A, TNRC6B, TNS3,
TOB2, TOMM7, TOP1, TOR1AIP2, TOX4, TPI1, TPM1, TPM3, TPM4, TPP1, TPR, TPT1,
TRA2A, TRA2B, TRAK1, TRANK1, TRIM31, TRIM38, TRIM56, TRIP11, TRIP12,
TSC22D1, TSPAN1, TSPAN3, TSPAN8, TSPO, TST, TTC3, TUBA1B, TUBA1C, TUBB4B,
TXN, TXNDC17, TXNIP, TXNRD1, U2AF1L5, U2SURP, UACA, UBA52, UBB, UBC, UBD,
UBE2D3, UBL5, UBN1, UBR2, UBR4, UBR5, UBXN4, UGP2, UGT2B17, UPF2, UQCR10,
UQCRB, UQCRC1, UQCRH, UQCRQ, USH1C, USMG5, USP15, USP16, USP33, USP34,
USP36, USP53, USP8, USP9X, UTRN, VAMP8, VAPA, VASP, VCP, VDAC1, VEGFA, VIL1,
VIM, VMP1, VPS13A, VPS28, VPS35, VPS37B, VPS4B, VTI1B, WAC, WAPL, WARS,
WASF2, WASHC2A, WASHC2C, WASL, WDR1, WDR60, WDR66, WIPF1, WIPF2, WNK1,
WSB1, XAF1, XBP1, XDH, XIAP, XRN1, XRN2, YIPF4, YME1L1, YTHDC1, YWHAB,
YWHAE, YWHAH, YWHAZ, YY1, ZBTB7A, ZC3H11A, ZC3H13, ZC3H15, ZCCHC6,
ZEB2, ZFAND5, ZFC3H1, ZFP36, ZFP36L1, ZFP36L2, ZFP91, ZG16, ZKSCAN1, ZMAT2,
ZMYND8, ZNF106, ZNF207, ZNF217, ZNF292, ZNF302, ZNF326, ZNF511PRAP1, ZNF622,
ZNF638, ZNF91, ZNFX1, ZNHIT1, ZRANB2, ZSWIM6, ZZEF1

The following examples are presented in order to more fully illustrate some embodiments of the invention. They should, in no way be construed, however, as limiting the broad scope of the invention.

### EXAMPLES

### Methods

Described below are experimental outlines and methods used in the studies described in Examples 1-2 below. Additional outlines, test subjects and methods, are further described in Examples 3-5, as well as modifications from the below methods, as indicated therein.

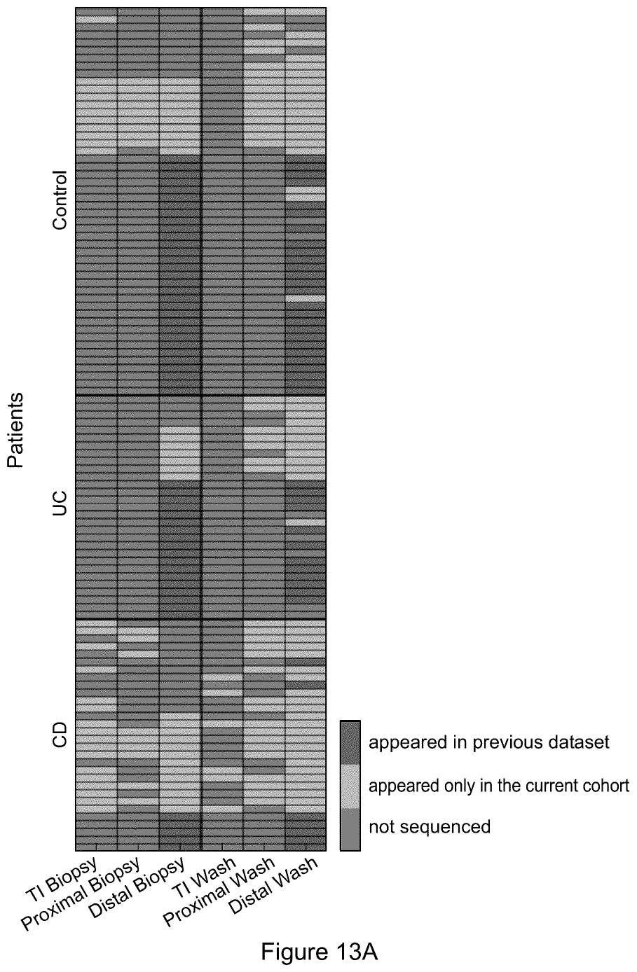

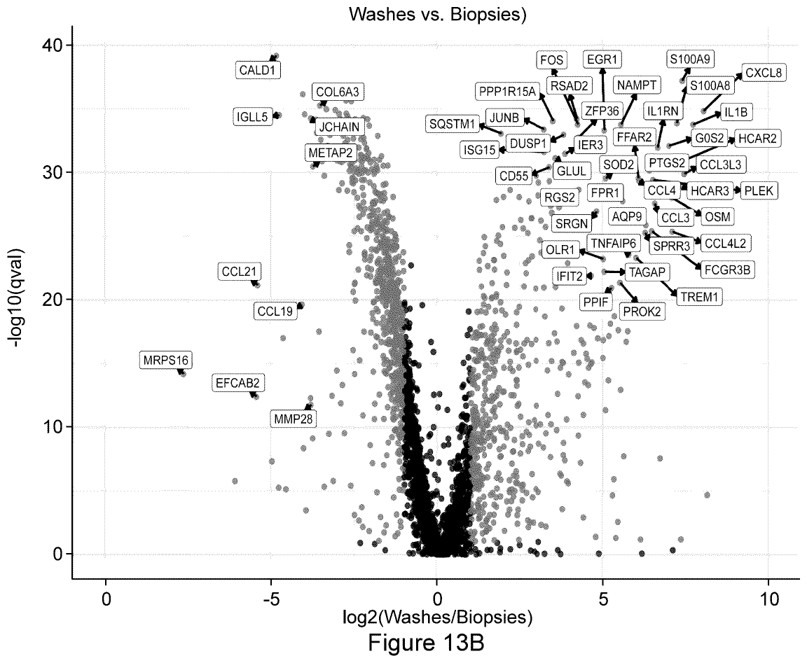

In a cross-sectional study, patients undergoing a lower endoscopy were recruited. The study group consisted of 56 patients and controls from a previously described cohort and 53 newly recruited patients. The 53 newly recruited patients comprised 19 controls, 11 ulcerative colitis (UC) patients, and 23 Crohn's disease (CD) patients. In total, in the experiments described in Examples 1-2, 24 ileal (involving TI)/ileocolonic CD patients were compared with a total of 35 colitis patients (comprising 29 UC and 6 Crohn's colitis patients) and 50 control non-IBD patients. All control patients had a lower endoscopy performed for unrelated reasons (colorectal screening, and so forth), and patients with IBD underwent the procedure because of clinical indications as per the decision of their treating physician irrespective of the present study. Five UC patients from the previous cohort were excluded because they had a sigmoidoscopy performed, as opposed to a full colonoscopy (FIG. 13A), because sigmoidoscopies only provide information about the distal colon. All IBD patients had colonoscopies spanning the TI to the distal colon, enabling annotation of the precise segment of inflammation. Clinical and demographic parameters were obtained from questionnaires at the time of enrollment and from patients' computerized medical records (Table 5). Additional patients and test subjects are described in Examples 3-5 below.

TABLE 5
Demographic and clinical characteristics of study
participants in Examples 1-2

| | | | Ulcerative |
|---|---|---|---|
| | Control | Crohn's disease | colitis |
| N | 50 (31) | 30 (7) | 29 (18) |
| Female gender, n (%) | 26 (52) | 14 (46.6) | 14 (48.2) |
| Age, years-median | 53.5 (34- | 33.5 (26.2-51) | 55 (43-68) |
| (IQR) | 70.7) | | |
| Age at diagnosis- | * | 29 (22.2-38.7) | 34 (20-43) |
| median (IQR) | | | |
| Weight, kg-median | 72 (63.6- | 67.5 (56-75) | 69 (53.7-85.7) |
| (IQR) | 81.1) | | |
| Smoking at | 6 (12) | 5 (16.6) | 2 (6.9) |
| induction, n (%) | | | |
| Past intestinal | 0 (0) | 2 (6.6) | 3 (10.3) |
| resection, n (%) | | | |
| Concomitant | 28 (56) | 5 (16.6) | 12 (41.3) |
| illnesses, n (%) | | | |
| Disease location, | * | Ileal-16 (53.3) | Proctitis-1 (3.4) |
| n (%) | | Colonic-6 (20) | Left-sided |
| | | | colitis-13 |
| | | | (44.8) |
| | | Ileo-colonic-8 | Pancolitis-15 |
| | | (26.6) | (51.7) |
| Concomitant biological | 0 (0) | 7 (23.3) | 6 (20.6) |
| therapy, n (%) | | | |
| Concomitant | 0 (0) | 0 (0) | 1 (3.4) |
| Immunomodulator | | | |
| therapy, n (%) | | | |
| Concomitant 5-ASA | 0 (0) | 4 (13.3) | 13 (44.8) |
| therapy, n (%) | | | |
| Concomitant steroidal | 0 (0) | 1 (3.3) | 3 (10.3) |
| therapy, n (%) | | | |
| Calprotectin level | * | 110 | 682 |
| (mcg/g), median | | (54.7-274.5) | (84.7-1000) |
| (IQR)** | | | |
| Endoscopic | * | 23 (76.6) | 16 (55.1) |
| inflammation, n (%)+ | | | |
| Histological | * | 21 (70) | 22 (75.8) |
| inflammation, n (%)++ | | | |

IQR-Interquartile range; 5-ASA-5-aminosalicyclic acid
* Not applicable for control patients
**Calprotectin levels were not available for all patients included. They were measured for patients with apparent inflammation on colonoscopy
+Endoscopic inflammation defined as appearance of ulcers/mucosal inflammation on colonoscopy
++Histological inflammation was defined as active histological disease activity as per the pathologist's assessment

Sample Collection-On colonoscopy, biopsy specimens (2 consecutive biopsy specimens per patient, i.e., double bite) were obtained from the designated areas: distal colon (left, sigmoid), proximal colon (right, between hepatic flexure and cecum), and TI, and fecal fluid was suctioned from the same areas. Fecal washes were obtained while advancing the scope (before any through-the-scope washing was applied) and biopsy specimens from corresponding areas were obtained during withdrawal. In patients with endoscopic inflammation of the colon/ileum, the biopsy specimens were obtained from the inflamed area, adjacent to ulcers. Samples were snap-frozen in liquid nitrogen and stored at −80° C. until further analysis.

Study Outcomes—The transcriptomic profiles of distal (left-sided) fecal washes obtained during the colonoscopy were assessed to determine their association with histologic inflammation in the distal colon/proximal colon/TI in the same patient. In addition, a comparison of transcriptomic profiles of fecal washes with transcriptomics of colonic/ileal biopsy specimens was performed. The cellular composition of distal vs proximal colonic and ileal fecal washes was also analyzed using computational deconvolution based on the single-cell RNA sequencing data. An analysis was further performed to identify gene modules characterizing patients with primary nonresponse to several therapeutic agents targeting IBD.

Exclusion Criteria—The exclusion criteria were as follows: age younger than 18 years, undetermined diagnosis of UC or CD (IBD unclassified), missing clinical/demographic data, and patients undergoing sigmoidoscopy/failing to complete a full colonoscopy in UC/ilcocolonoscopy in CD.

Definition of Endoscopic Mucosal Healing and Histologic Remission-Endoscopic and histologic inflammation were graded according to standardized indices and by blinded gastroenterologists and pathologists, respectively. Endoscopic scores were determined prospectively during colonoscopy. Mucosal healing was defined as an absence of ulcers and a lack of inflammation on endoscopic examination, for CD and UC, respectively.

Histologic inflammation was determined by a certified pathologist based on biopsy specimens from the same colon (distal/proximal) or ileal (TI) region used for the biopsy transcriptomics. Assessment of histologic disease activity was performed using the Nancy Score for UC and the Global Histological Disease Activity Score for CD. Because histologic score assessment has not been fully standardized in the previous literature for CD, a dichotomous assessment only was performed (active histologic disease activity/lack of histologic disease activity).

Calprotectin Measurement-Calprotectin was assessed using a previously established Quantum-blue fCAL extended point-of-care testing assay (Bülmann, Schönenbuch, Switzerland). Calprotectin levels were measured prospectively from fecal washes at the beginning of each colonoscopy. Calprotectin was measured only in patients with apparent inflammation on colonoscopy.

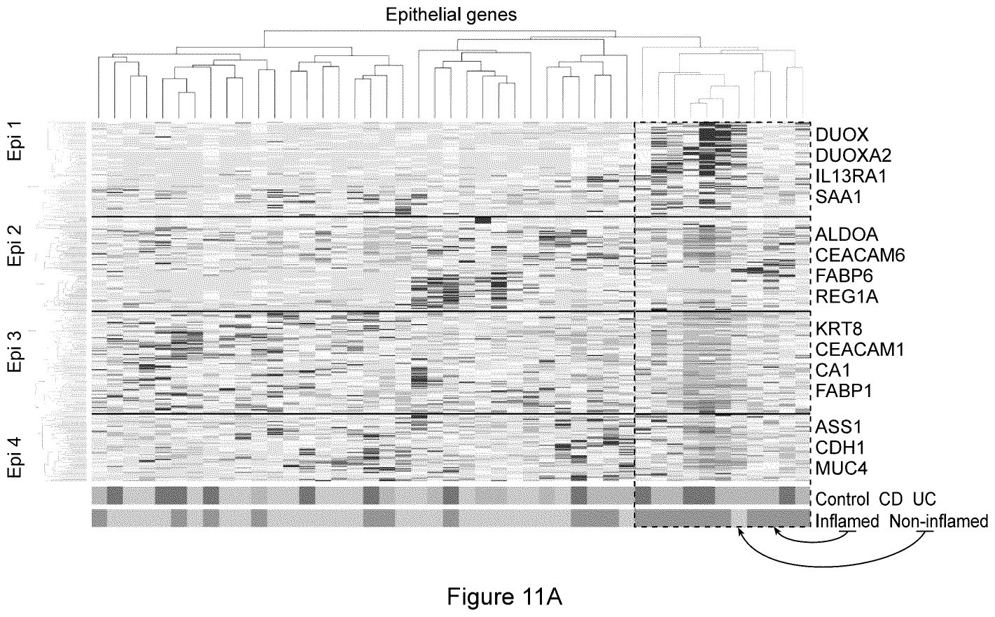

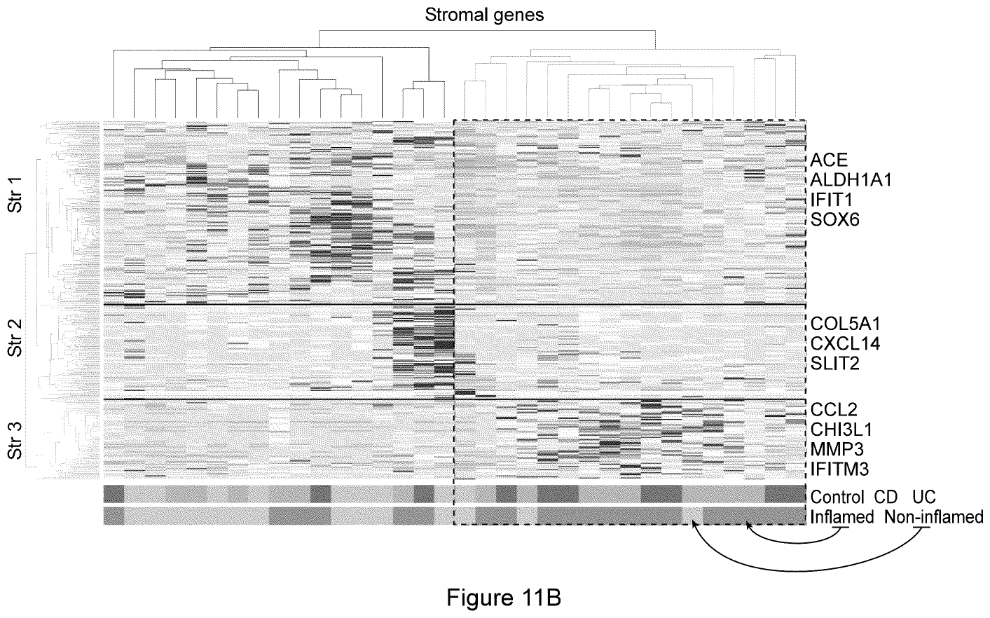

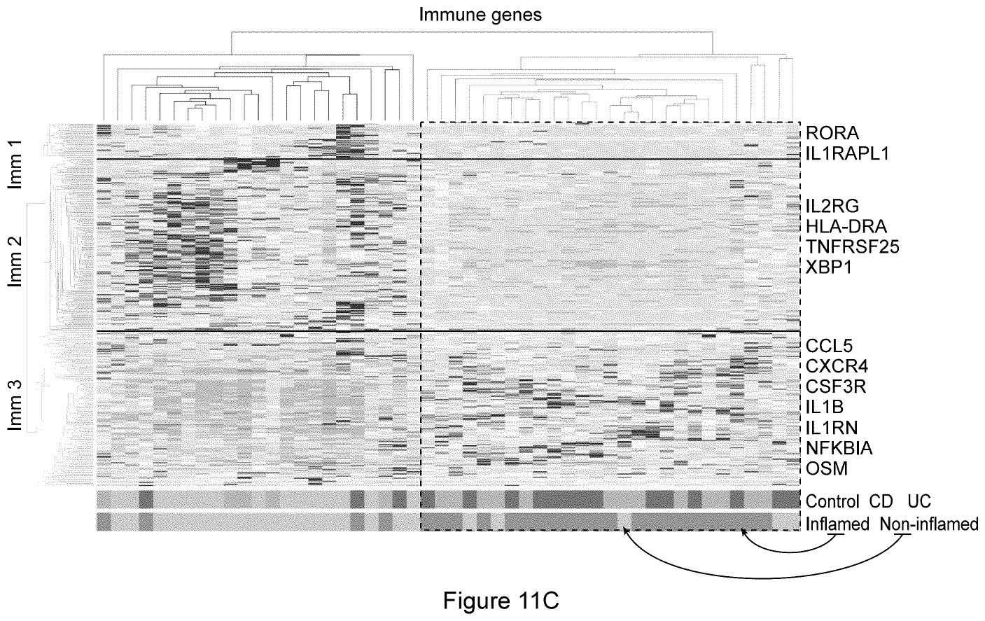

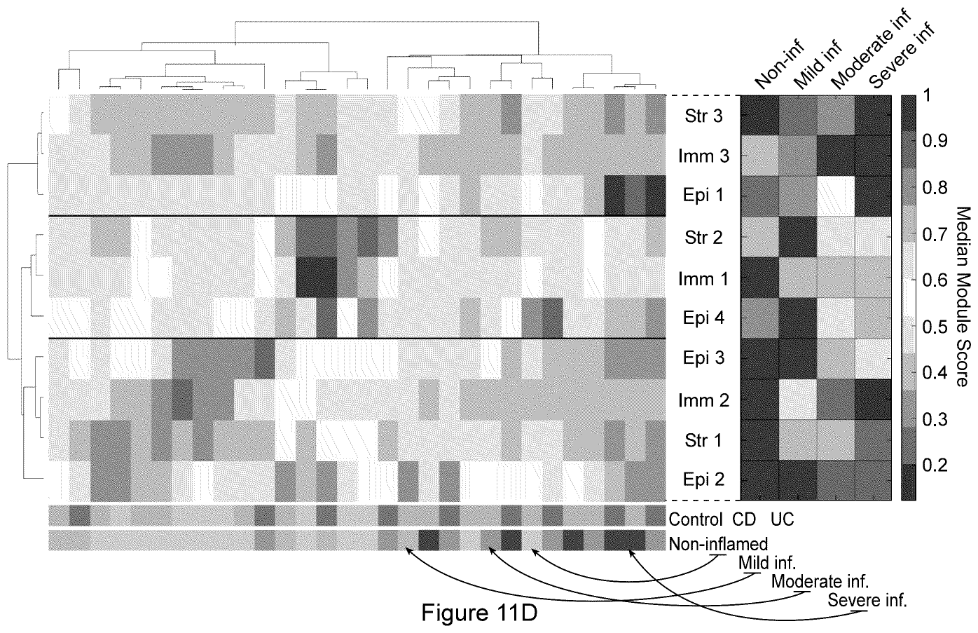

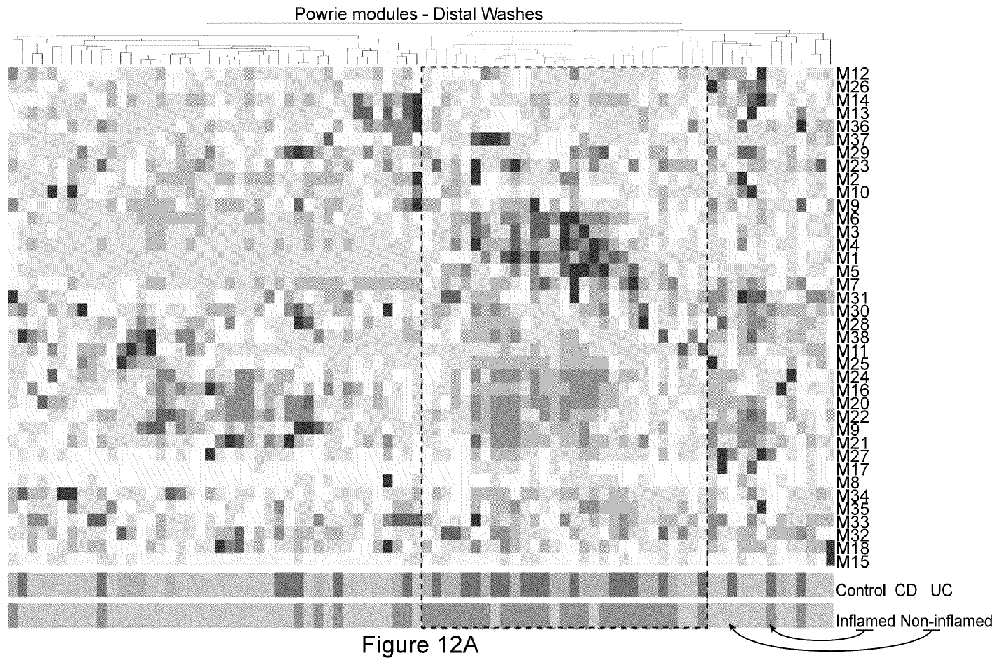

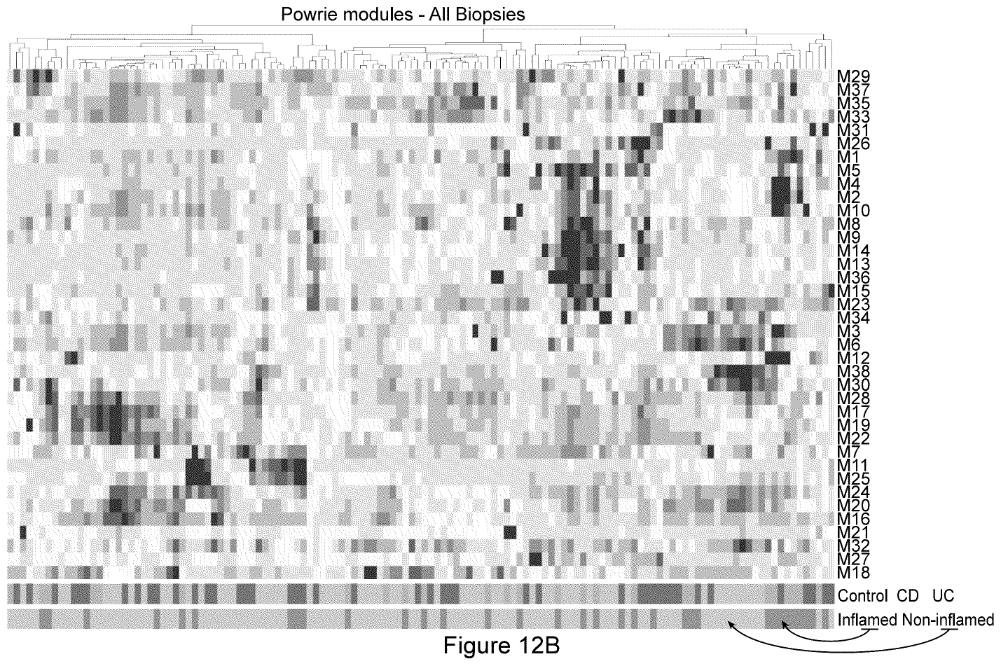

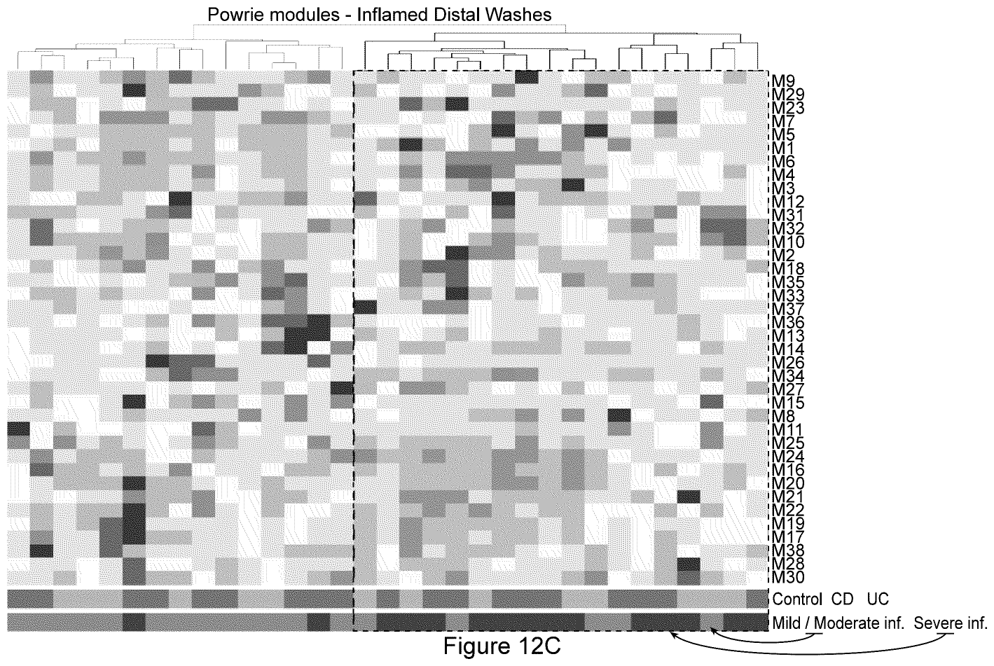

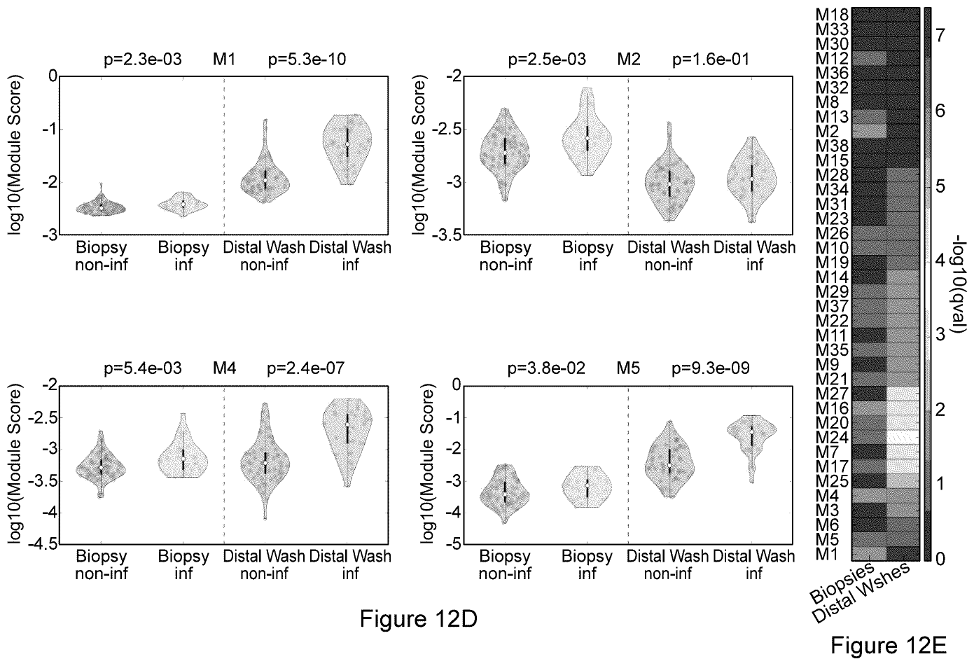

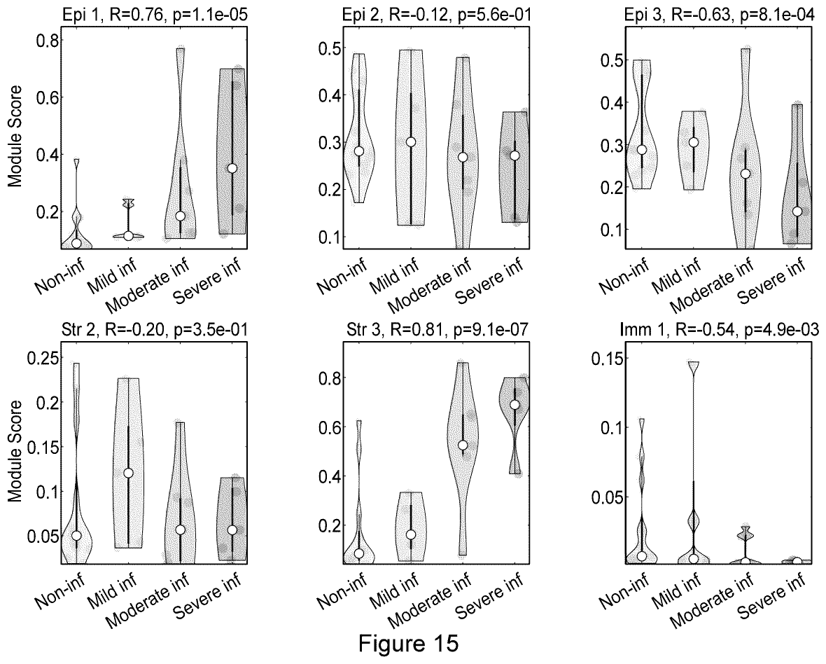

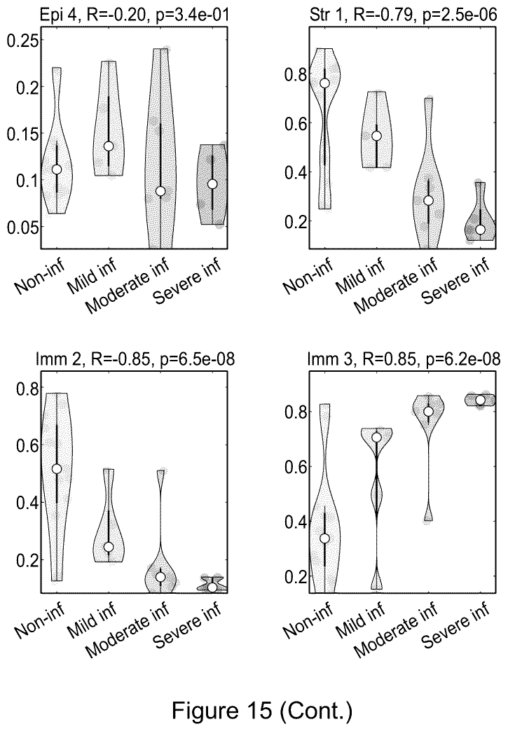

Disease Severity Assessment—The disease severity analysis was based on several parameters. The first parameter was the endoscopic score (Mayo endoscopic score of 0 to 3 for UC and the Simple Endoscopic Score for CD, with scores stratified per endoscopic remission/mild/moderate/severe, 0-3). The threshold for mucosal healing (lack of endoscopic inflammation) was defined as no ulcers or mucosal inflammation. This annotation was performed by a gastroenterologist blinded to the bioinformatic analysis. The second parameter was the fecal wash calprotectin levels (meg/g). Calprotectin values were stratified as accepted, normal (<100; score, 0), mild to moderate (100-300; score, 1) and severe (>300; score, 2). The third parameter was the previous use of steroids, with a score of 0 or 1. The fourth parameter was previous events of IBD exacerbation leading to hospitalization, with a score of 0 or 1. An inflammation severity score was calculated as the mean of these 4 values for each patient in the severity analysis (Table 6). In FIGS. 11D and 15, patients were binned into 4 groups based on equal percentile thresholds of the severity scores of patients with inflammation. In FIG. 12C, patients were classified into 2 groups according to their values less than/greater than the median severity score (0.67).

TABLE 6
Disease severity parameters and scores of inflamed distal washes

| | | | | | SESCD/ | |
|---|---|---|---|---|---|---|
| | | | | | MAYO | |
| Inflamed | | Calp. class | Hospitalization | Steroids | endoscopic | Severity |
| Distal Washes | Calp. | (0-2) | (no 0, yes 1) | (no 0, yes 1) | scores (0-3) | score |
| 243WNOV | 30 | 0 | 0 | 0 | 1 | 0.25 |
| 109W | NaN | NaN | 0 | 0 | 1 | 0.33 |
| 82W | 763 | 2 | 0 | 0 | 0 | 0.50 |
| 32W | 100 | 1 | 1 | 0 | 0 | 0.50 |
| 36W | NaN | NaN | 0 | 0 | 0 | 0.00 |
| 241-1WNOV | 45 | 0 | 1 | 1 | 1 | 0.75 |
| 87W | 75 | 0 | 0 | 0 | 0 | 0.00 |
| 268W | NaN | NaN | 0 | 0 | 0 | 0.00 |
| 539W | 65.8 | 0 | 0 | 0 | 1 | 0.25 |
| 401W | 30 | 0 | 0 | 0 | 1 | 0.25 |
| 29W | NaN | NaN | 0 | 0 | 1 | 0.33 |
| 451W | 30 | 0 | 0 | 0 | 1 | 0.25 |
| 221W | 214 | 1 | 0 | 0 | 1 | 0.50 |
| 225W | 176 | 1 | 0 | 1 | 1 | 0.75 |
| 320W10R | NaN | NaN | 0 | 0 | 2 | 0.67 |
| 250W | NaN | NaN | 0 | 1 | 1 | 0.67 |
| 124W | 545 | 2 | 0 | 0 | 2 | 1.00 |
| 312W | 1000 | 2 | 0 | 1 | 2 | 1.25 |
| 215W | 1300 | 2 | 0 | 1 | 3 | 1.50 |
| 50W | 222 | 1 | 0 | 0 | 0 | 0.25 |
| 306W | 1000 | 2 | 1 | 1 | 3 | 1.75 |
| 520W | 110 | 1 | 1 | 0 | 3 | 1.25 |
| 40W | 506 | 2 | 1 | 1 | 3 | 1.75 |
| 541W | 45 | 0 | 0 | 1 | 3 | 1.00 |
| 399W | 601 | 2 | NaN | NaN | 2 | 2.00 |
| 81W | NaN | NaN | 0 | 1 | 1 | 0.67 |
| 524W | 58 | 0 | 1 | 0 | 1 | 0.50 |
| 512W | NaN | NaN | 1 | 0 | 3 | 1.33 |
| 246W | 110 | 1 | 0 | 0 | 3 | 1.00 |
| 66W | NaN | NaN | NaN | NaN | 2 | 2.00 |
| 41W | 39 | 0 | 0 | 0 | 1 | 0.25 |
| 42W | 1000 | 2 | 1 | 1 | 3 | 1.75 |
| 352W | 432 | 2 | 0 | 0 | 3 | 1.25 |

SESCD—Simple Endoscopic Score for Crohn's Disease.
Calp.—Calprotectin

RNA Extraction—For colonic biopsy specimens, snap-frozen tissues (2×2 mm) were thawed in 300 μL Tri-reagent (Sigma Aldrich) and homogenized mechanically with bead beating, followed by a short centrifugation step to pull-down beads and any tissue leftovers. For fecal washes, Tri-reagent was added at a ratio of 3:1, samples were allowed to thaw on ice, followed by thorough mixing. A first centrifugation step was used (1 min, 18,000 rpm) to eliminate fecal solids. After this, ethanol was added at a ratio of 1:1 to the supernatant from the previous step and continued according to the manufacturer's instructions of the Direct-zol Mini and Micro Prep Kit (R2052; ZYMO Research).

Bulk RNA Sequencing of Samples-RNA was processed by the mcSCRBseq protocol with minor modifications. For biopsy specimen RNA, a reverse-transcription (RT) reaction was applied to 10 ng total RNA. For fecal wash RNA, the RT reaction was started with one third of the total eluted volume with a final reaction volume of 20 μL (1× Maxima H Buffer, 1 mmol/L deoxynucleoside triphosphate, 2 μmol/L TSO* E5V6NEXT, 7.5% PEG8000, 20 U Maxima H enzyme, and 2 μL barcoded RT primer). Subsequent steps were applied as mentioned in the protocol. The library preparation was performed using the Nextera XT kit (Illumina) on 0.6 ng amplified complementary DNA. The library final concentration of 2 nmol/L was loaded on a NovaSeq 6000 (Illumina) sequencing machine that aimed for 10-20 million reads per sample with the following setting: Read1, 16 bp; Index1, 8 bp; and Read2, 66 bp.

Bioinformatics and Computational Analysis-Illumina output sequencing raw files were demultiplexed to FASTQ files using the bcl2fastq package. To obtain the Unique Molecular Identifier (UMI) counts, FASTQ files were aligned to the human reference genome (GRCh38.91) using the zUMI package. Statistical analyses were performed with MATLAB R2022a. Mitochondrial genes and non-protein-coding genes were removed from the analysis. Protein-coding genes were extracted using the annotation in the Ensembl database (BioMart) for reference genome GRch38 version 91, using the R package biomaRt (version 2.44.4). For the initial analyses described in Example 5, gene expression for each sample consequently was normalized by the sum of the UMIs of the remaining genes that individually took up less than 10% of the total UMI sum. Clustering and principal component analysis were performed in MATLAB using the Z-score-transformed expression matrix. Clustering was performed with the MATLAB function clustergram, using Spearman correlation distances. Differential gene expression was performed using Kruskal-Wallis tests and Benjamini-Hochberg false discovery rate (FDR) corrections. Computational deconvolution was performed using CIBERSORTx using signature tables obtained from a single-cell atlas of the human colon. Original cell-type annotations were used, but subsequently coarse-grained into a small number of cell types. M cells were removed from the analysis because of their low abundance.

### Classifier of Inflammation

For the initial analyses described in Example 5, samples used to build the classifier were fecal washes or biopsy specimens taken from the distal segment of the colon, with total UMI counts higher than 10,000. Classification was based on a parameter that represented the summed expression of inflammation-related genes, extracted as follows. For 40 iterations, the data set was split into a training set (70%) and a test set (30%). Differential gene expression between the inflamed and noninflamed training set samples was calculated. Genes with a maximal normalized expression higher than 10⁻⁴ over all the included samples that were expressed in more than 5% of the samples were further considered. The Kruskal-Wallis test followed by the Benjamini-Hochberg FDR correction was performed. Genes whose mean expression in inflamed samples was more than 2-fold higher than the mean in noninflamed samples, with FDR q-value less than 0.1 were selected as inflammatory markers. Inversely, genes whose mean expression in inflamed samples was less than 2-fold lower than the mean in noninflamed samples, with FDR q-value less than 0.1 were chosen as noninflammatory markers. Those genes were identified based on the training set only.

Next, for the test set samples, the sums of inflammatory markers and noninflammatory markers were calculated, after the gene expression levels for each gene were normalized internally by their maximal values across the test set. An inflammation score was calculated for each test set sample, as follows: (sum of normalized inflammation markers)/(sum of normalized inflammatory markers+ sum of normalized noninflammatory markers). The receiver operating characteristic curve and subsequent false-positive rate, true-positive rate, and AUC were recorded for each test set to examine the classification of samples of inflamed patients and noninflamed patients based on the inflammation score.

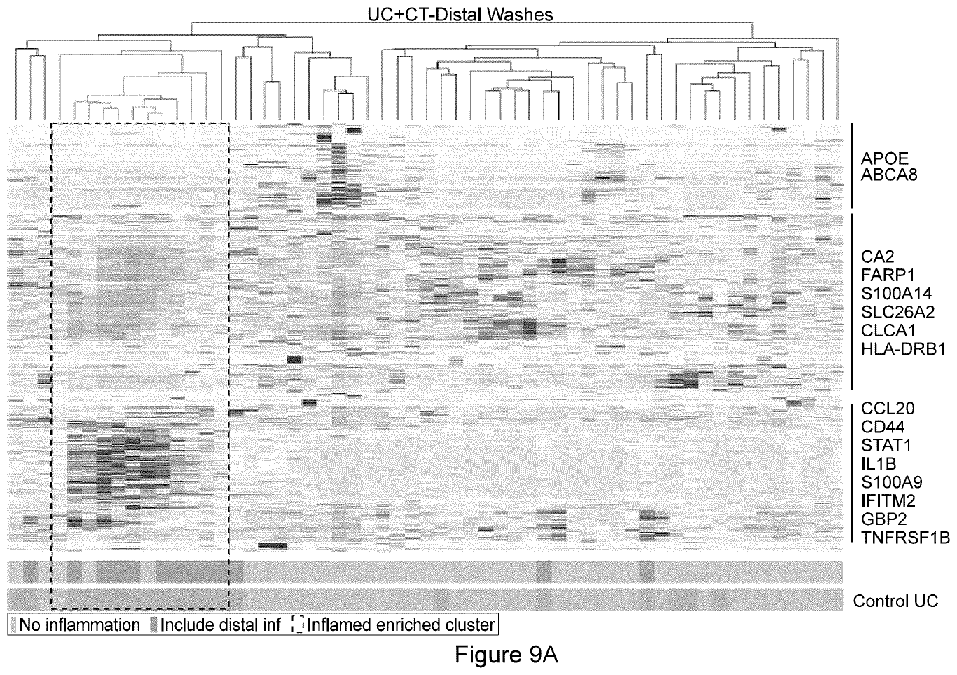

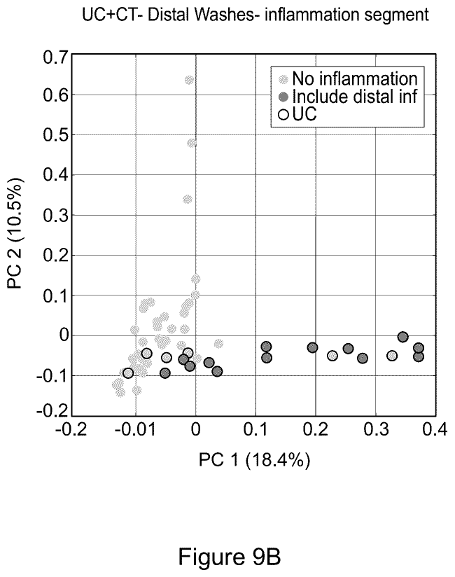

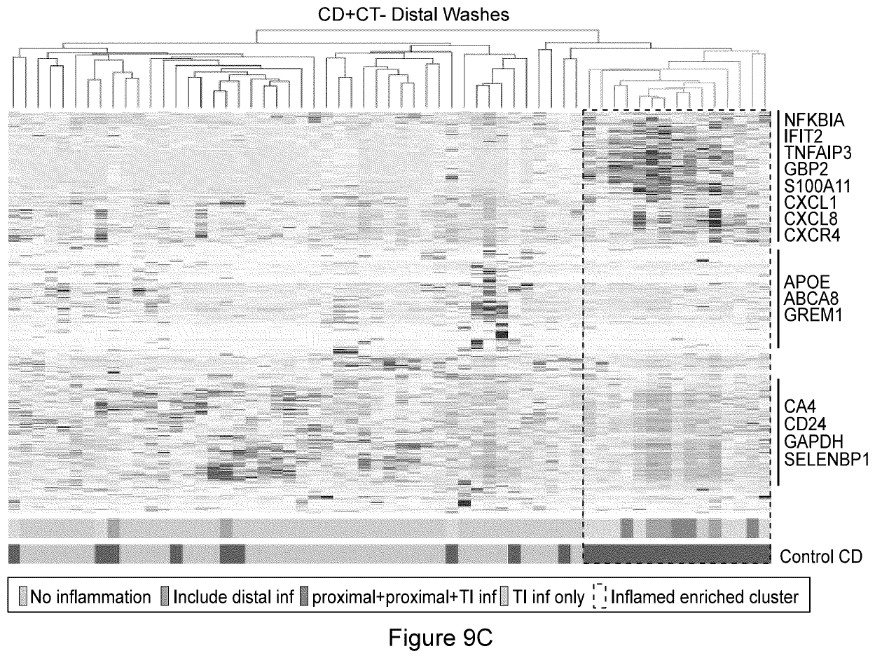

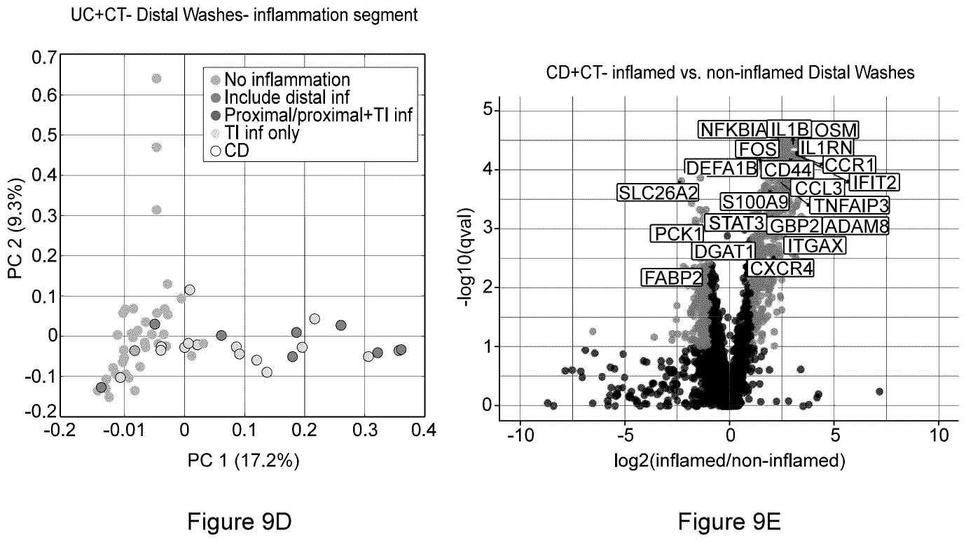

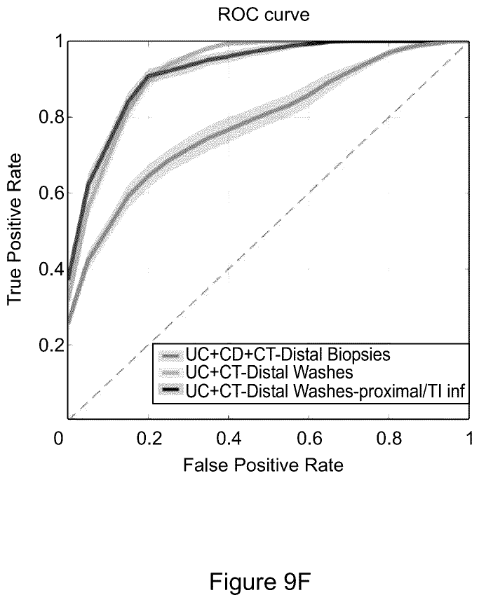

The division of the data set into training and test sets was performed randomly 40 times. False-positive rates over all 40 iterations were binned into 20 equal intervals (0-0.05, 0.05-0.1 . . . 0.95-1), and the means and standard errors (SEs) of the false-positive rates and the corresponding true-positive rates over each bin were calculated (FIG. 9F).

This analysis was performed 3 times to classify different types of groups: the first classification was performed on distal fecal washes of control and UC patients, and the classification criterion was either histologic inflammation in the distal and/or proximal segment of the colon or no inflammation at all. The second classification was performed on distal fecal washes taken from control and CD patients, with the classification criterion of either histologic inflammation in the proximal colon and/or TI, whereas the distal segment was not histologically inflamed or no histologic inflammation at all. The third analysis was performed on distal biopsies. Biopsy specimens taken from the distal colon of controls, CD patients, and UC patients were used to classify histologic inflammation or no inflammation in the distal colon. In case no markers passed the differential gene expression thresholds, the iteration was skipped for the specific training and test set. Receiver Operating Curves analysis was also carried out to compute the Area Under the Curve metric for the classification of active inflammation based on stool samples.

### Gene Modules

To extract gene modules based on specific intestinal cell types (as described in Example 5), a single-cell atlas of the human colon was used to extract signatures of 3 cell types: immune, stroma, and epithelia. Expression levels of these coarse-grained cell types were determined as the maximum over the average expression of the single-cell RNA sequencing cluster of cell types belonging to the coarse-grained cell type. A signature was defined for each one of the coarse-grained cell types by including genes with an expression level above 5×10⁻⁶ of the UMI sum and that were expressed at least 3 times higher than any other coarse-grained cell type.

Next, the signature genes of each coarse-grained cell type in the data set was found, and specifically included the fecal washes taken from the distal colon (FIGS. 11A-11E). For each cell type, expression of each signature gene was divided by the sum of the cell-type-specific signature genes. This yielded 3 tables, representing the fraction of each signature gene from the total sum of the cell type-specific signature genes. Clustering analysis was performed on the Z-score of the coarse-grained cell type tables, including samples with a UMI count higher than 5000 for epithelial genes, 2000 for stromal genes, and 2000 for immune genes and including genes expressed above 2×10⁻³ of the summed expression of cell type-specific genes in at least 1 sample. Signature genes were classified manually into 4 epithelial (FIG. 11A), 3 stromal (FIG. 11B), and 3 immune (FIG. 11C) modules, based on the clustergram. The score of each module in each sample was calculated as the sum of the expression of genes belonging to the same module (from the signature tables renormalized over the sum of the respective cell type-specific signatures). The module scores of all samples were standardized (Z-score) and clustered using the MATLAB clustergram function with Spearman correlation distances (FIG. 11D). To this end, samples that both individually passed the cell type-specific UMI sums of the signature genes and had a total UMI count greater than 10,000 were included. Enrichr was used to identify pathways enriched in each of the modules (MSigDB Hallmark 2020 data set).

Results for the initial analyses are presented in Example 5. Results of subsequent analyses and diagnostic classifiers identified following subsequent analyses are provided in Examples 1-4 and 6.

### Example 1. Host Fecal Transcriptomics Characteristic of Terminal Ileal CD or Colonic CD

Further to the initial analyses described in Example 5, additional Analyses were performed to identify diagnostic and prognostic classifiers for characterizing GI inflammation. One of the analyses was directed to the identification of classifiers for special populations of patients afflicted with inflammatory bowel disorders, in particular patients with terminal ileal CD and patients with colonic CD.

The analyses were performed by identifying sets of genes that were significantly up-regulated both in colonic CD inflamed biopsies compared to terminal ileal (TI) inflamed biopsies, and also up-regulated in distal fecal washes of CD patients that had distal colonic inflammation and ones that had (TI) inflammation and no distal colonic involvement. The log2-fold changes of the biopsies and the fecal washes were multiplied to obtain a score, such that genes that were highly up-regulated in both biopsies and distal fecal washes of patients with distal colonic inflammation compared to TI inflammation had elevated positive scores, whereas genes that were highly down-regulated in both biopsies and distal fecal washes of patients with colonic inflammation compared to TI inflammation had a highly negative score. This score was sorted from smallest to largest and the extreme most negative and most positive 30 genes were selected as markers of TI or distal colonic inflammation respectively.

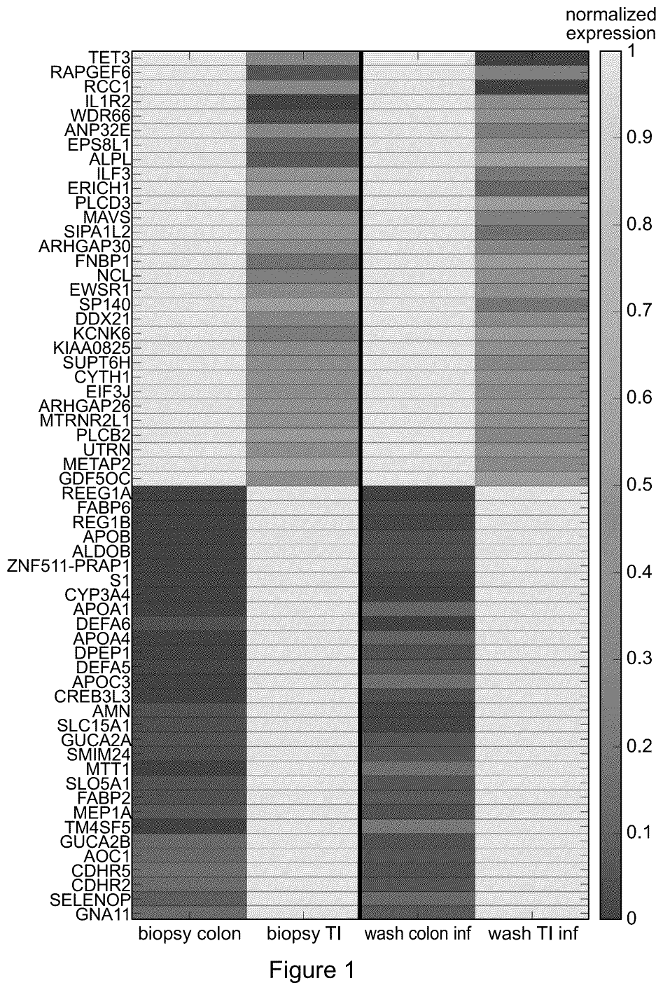

FIG. 1 is a heatmap presenting the results of the analysis. Each row is a gene, each column is a sample type, biopsies are from Crohn's patients with active inflammation, either in the colon (first column of each sample type) or terminal ileum (TI, second column of each sample type). Washes were all from the distal colon and originated from either patients with inflammation that includes the distal colon (third column) or TI inflammation with no distal colonic involvement (fourth column). Columns 1+2 are normalized to their maximum across these two biopsy locations, columns 3+4 normalized to their maximum across these two wash types.

As can be seen in FIG. 1 the following 30 gene products were specifically elevated in fecal samples of Crohn's patients afflicted with inflammation in the colon that does not involve the terminal ileum (herein designated “Group A gene products”):

### TET3, RAPGEF6, RCC1, IL1R2, WDR66, ANP32E, EPS8L1, ALPL, ILF3, ERICH1, PLCD3, MAVS, SIPA1L2, ARHGAP30, FNBP1, NCL, EWSR1, SP140, DDX21, KCNK6, KIAA0825, SUPT6H, CYTH1, EIF3J, ARHGAP26, MTRNR2L1, PLCB2, UTRN, METAP2, and GDF5OS.

Accordingly, elevated levels of one or more of the 30 transcripts above in fecal content are prognostic for inflammation in the colon that does not involve the terminal ileum. The summed normalized expression of these markers, denoted “sumCOLON”, was computed as the sum of normalized UMI counts of all of these genes. The normalized UMI counts are obtained for each sample and gene by dividing the UMI count of each gene by the sum of UMI counts of all genes in that sample.

In addition, FIG. 1 shows that the following 30 gene products were specifically elevated in fecal samples of patients afflicted with inflammation in the terminal ileum (herein designated “Group B gene products”):

### REG1A, FABP6, REG1B, APOB, ALDOB, ZNF511-PRAP1, SI, CYP3A4, APOA1, DEFA6, APOA4, DPEP1, DEFA5, APOC3, CREB3L3, AMN, SLC15A1, GUCA2A, SMIM24, MTTP, SLC5A1, FABP2, MEP1A, TM4SF5, GUCA2B, AOC1, CDHR5, CDHR2, SELENOP, GNA11.

Thus, Elevated levels of one or more of the 30 transcripts above in fecal content are prognostic for inflammation in the terminal ileum The summed normalized expression of these markers, denoted “sumTI”, was similarly computed as specified above.

For the construction of a diagnostic classifier, the variable sumCOLON/(sumCOLON+sumTI) was computed. This variable provides a score in the form of a unitless number between 0 and 1, wherein high values (close to 1) are indicative of colon inflammation and low values (close to 0) are indicative of ileal inflammation. The results indicate that accurate classification into terminal ileal CD or colonic CD can be performed by selecting a threshold based on the balance between specificity and sensitivity as in all classification tasks. Accordingly, the results demonstrate that the marker combinations identified above can accurately identify patients with ileal CD or colonic CD using supervised classification algorithms.

### Example 2. Improved Classifier for Determining the Severity of GI Inflammation

In addition, improved prognostic classifiers for determining the severity of GI inflammation were identified in the analyses. First, gene products correlated with specific endoscopic severity scores (MAYO for UC and SESCD for CD) were identified. Transcript UMIs of these gene products were then analyzed to identify prognostic classifiers. Remarkably, a prognostic classifier capable of determining the severity of inflammation in both UC and CD patients with high sensitivity and specificity was identified.

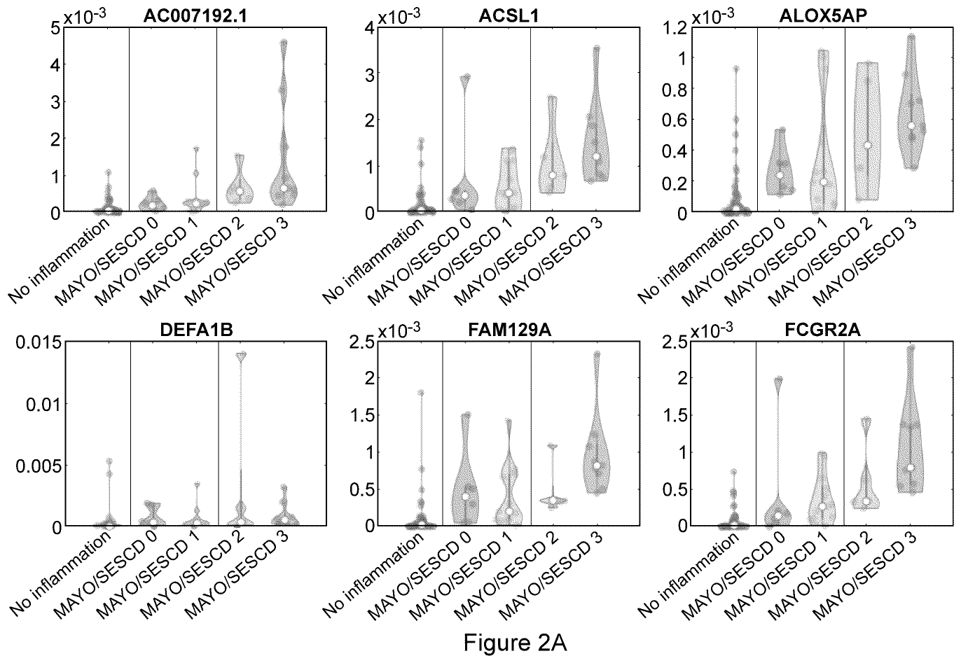

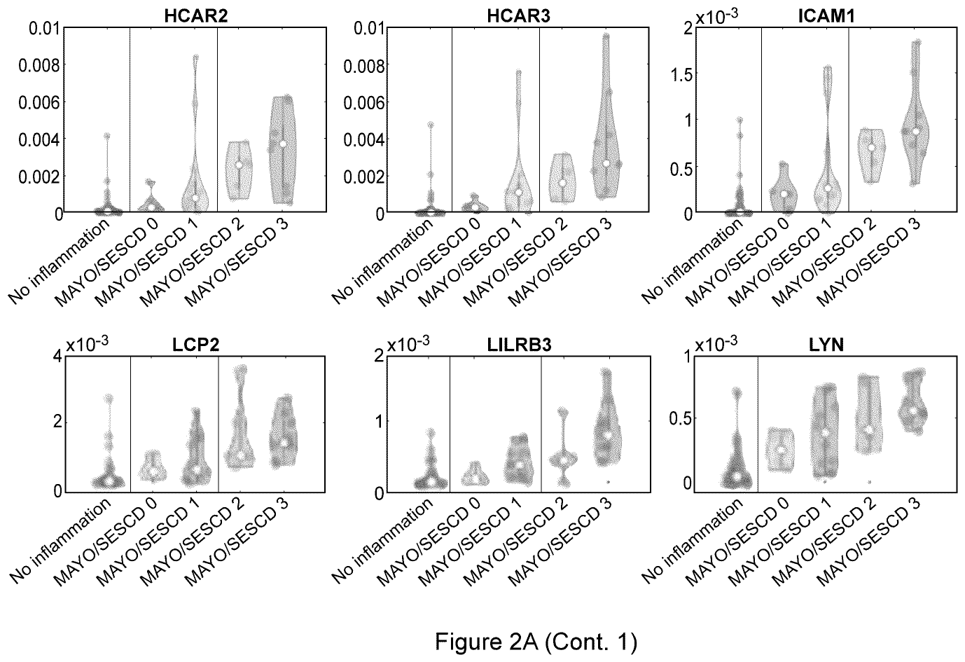

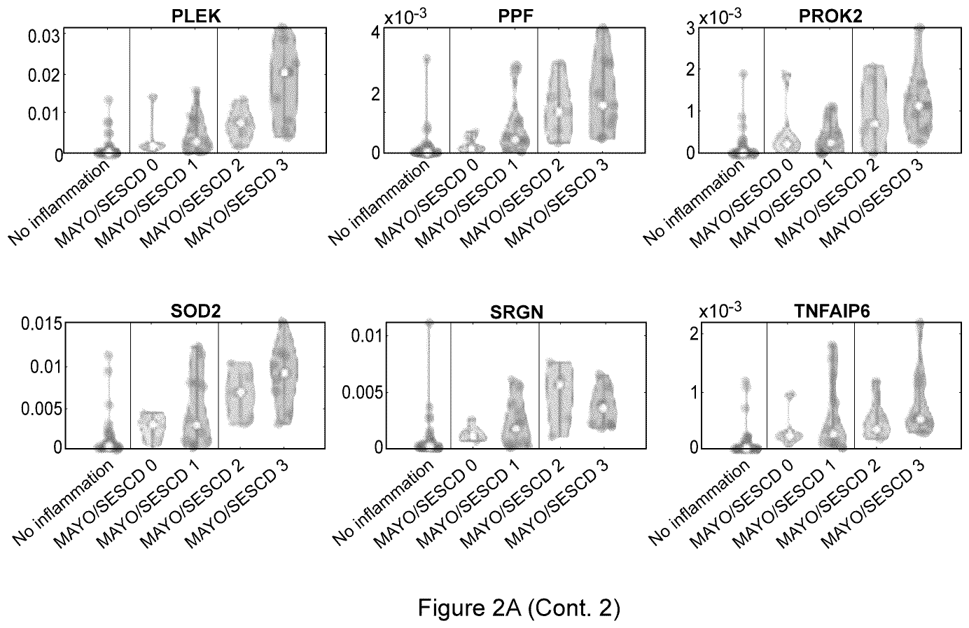

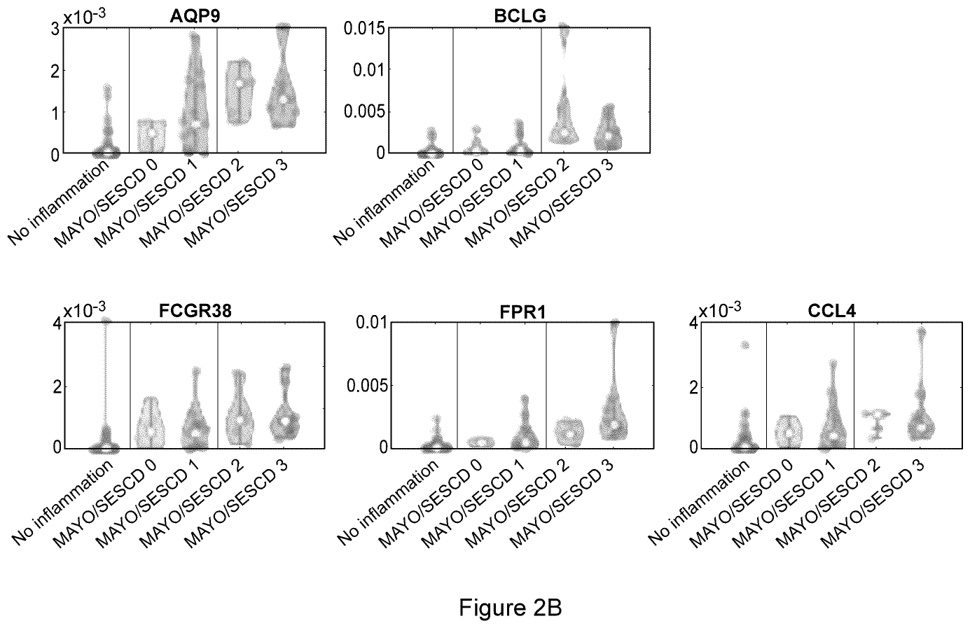

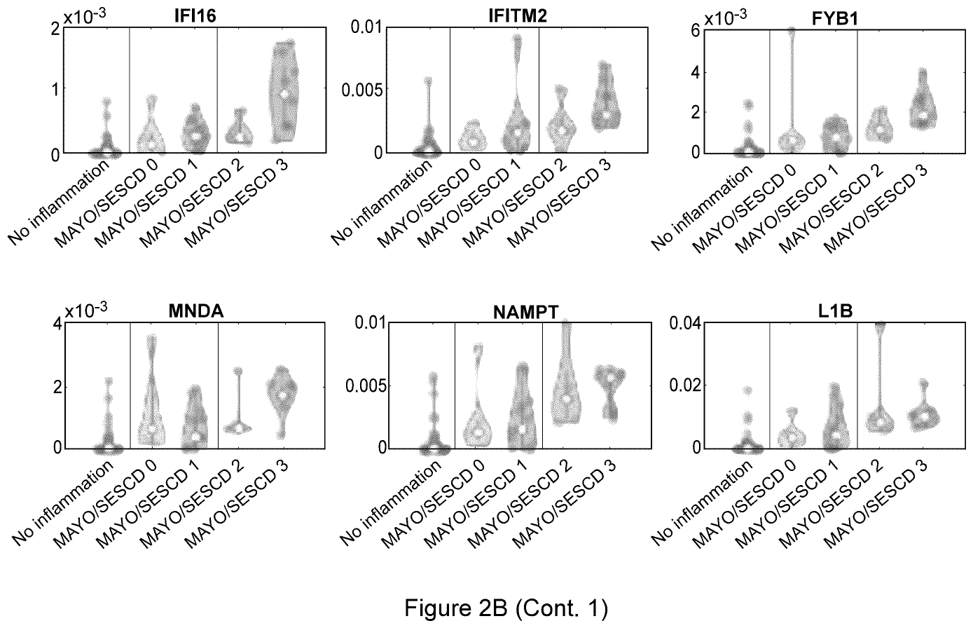

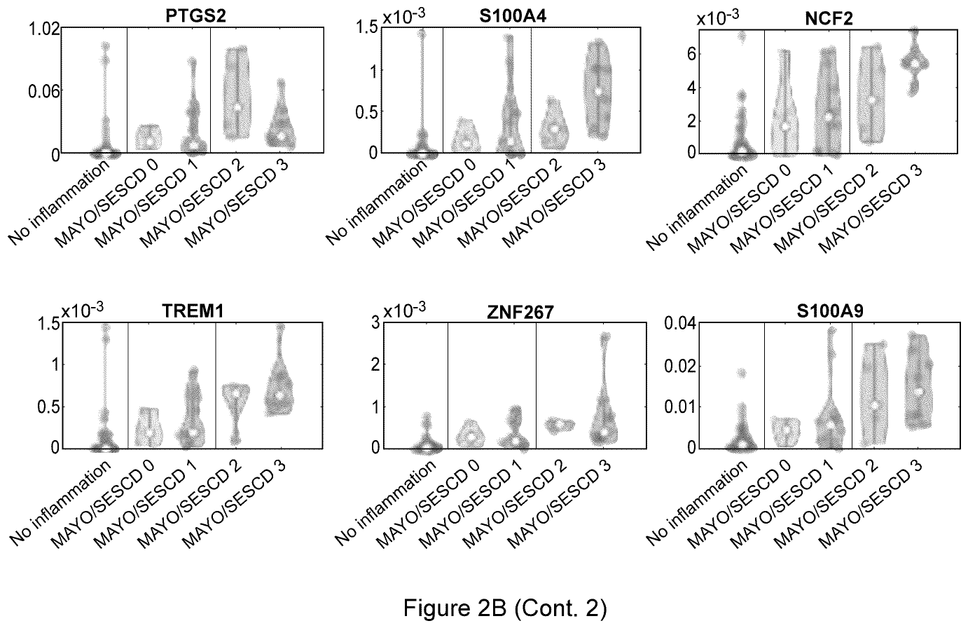

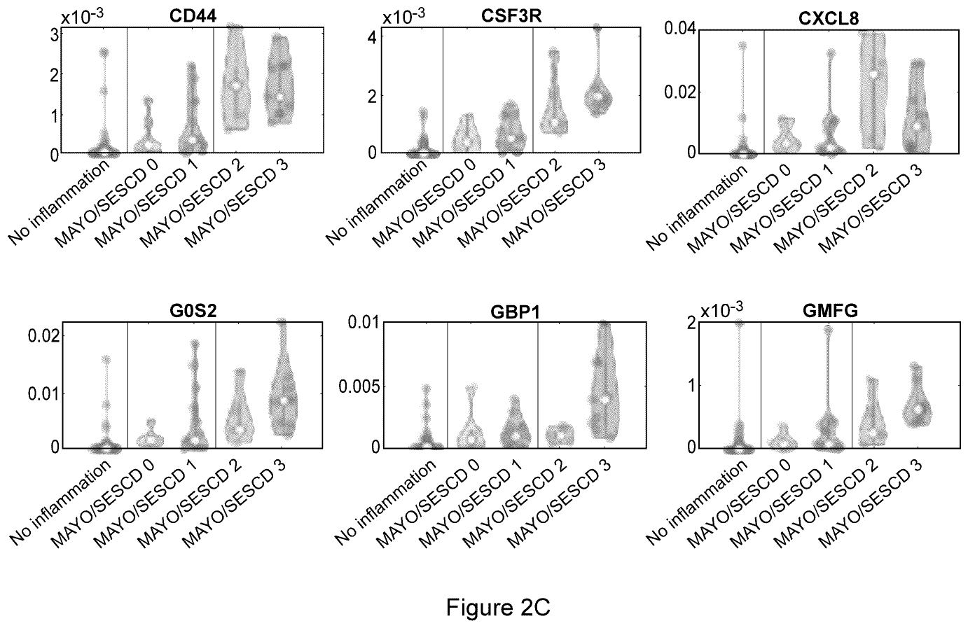

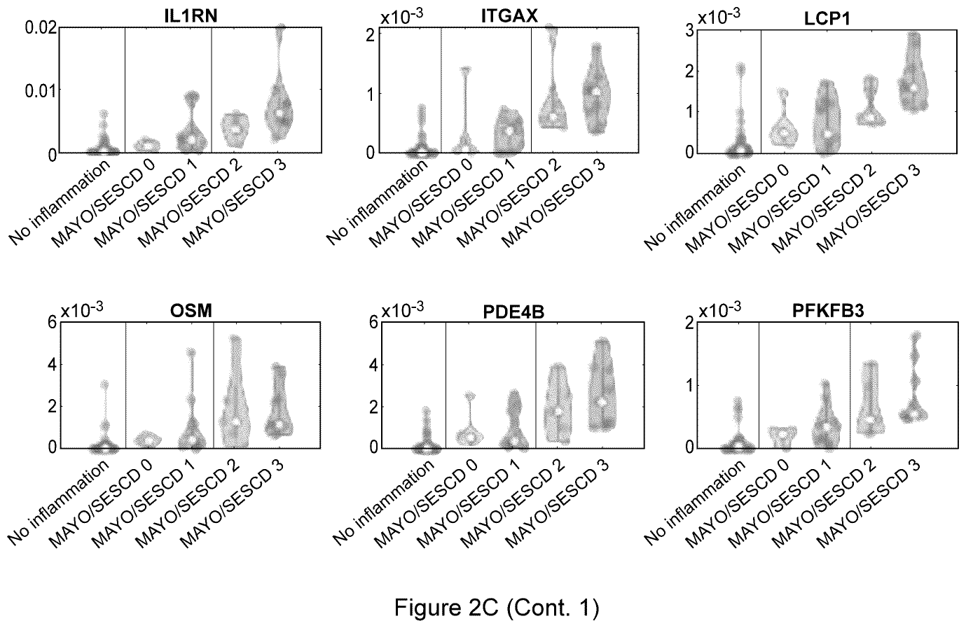

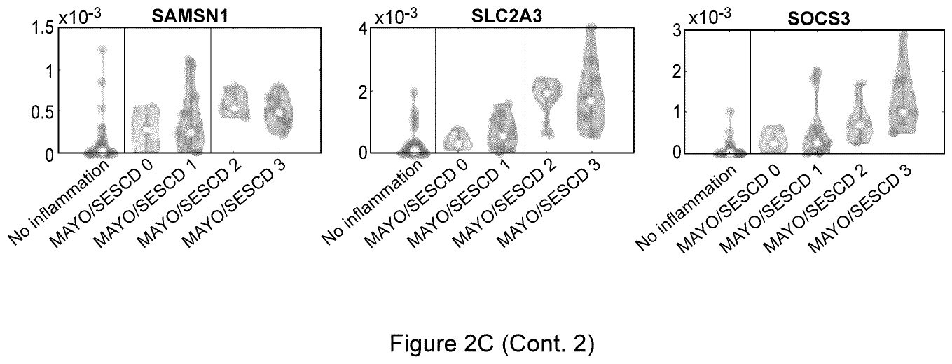

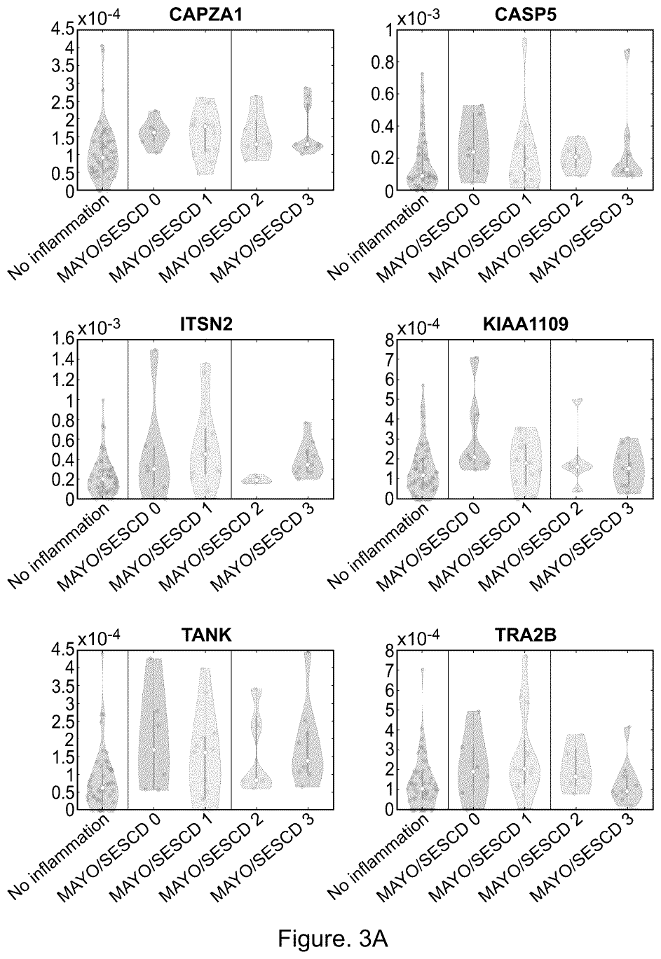

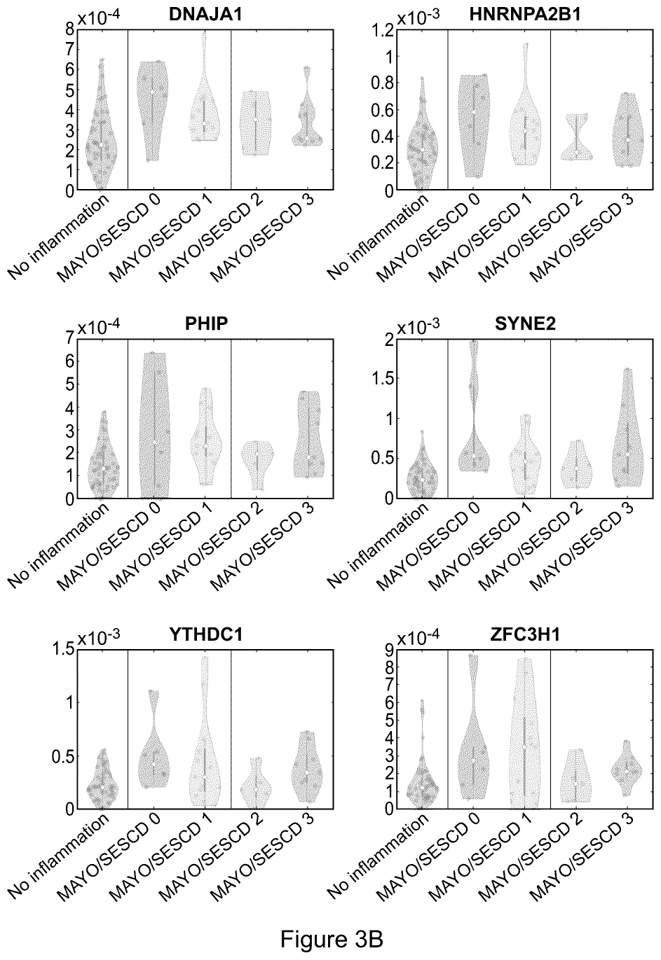

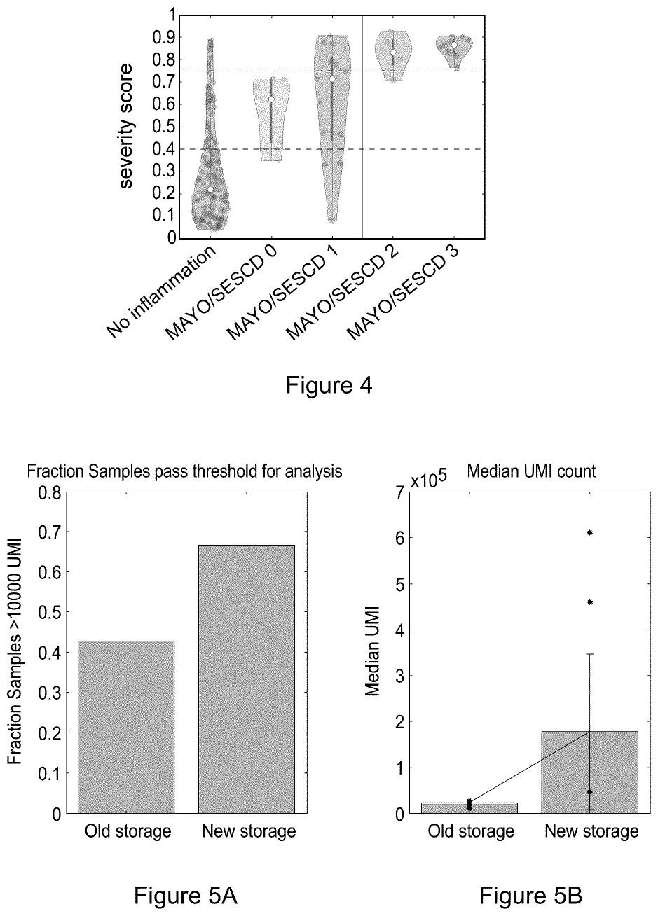

The results are presented in FIGS. 2-4, as detailed below. In particular, FIG. 2A shows the expression of the following gene products: AC007192.1, ACSL1, ALOX5AP (top left to top right), DEFA1B, FAM129A, FCGR2A (2nd row, left to right), HCAR2, HCAR3, ICAM1 (3^{rd} row, left to right), LCP2, LILRB3, LYN (4th row, left to right), PLEK, PPIF, PROK2 (5^{th} row, left to right), SOD2, SRGN, TNFAIP6 (bottom left to right). FIG. 2B shows the expression of: AQP9, BCL6 (top left to right), FCGR3B, FPR1, CCL4 (2nd row, left to right), IFI16, IFITM2, FYB1 (3^{rd} row, left to right), MNDA, NAMPT, IL1B (4^{th} row, left to right), PTGS2, S100A4, NCF2 (5^{th} row, left to right), TREM1, ZNF267, S100A9 (6^{th} row, left to right). FIG. 2C shows the expression of. CD44, CSFR3, CXCL8 (top left to right), GOS2, GBP1, GMFG (2^{nd} row, left to right), IL1RN, ITGAX, LCP1 (3^{rd} row, left to right), OSM, PDE4B, PFKFB3 (4^{th} row, left to right), SAMSN1, SLC2A3, and SOCS3 (5^{th} row, left to right). FIG. 3A shows the expression of: CAPZA1, CASP5 (top left to right), ITSN2, KIAA1109 (2^{nd} row, left to right), TANK, TRA2B (bottom, left to right); FIG. 3B shows the expression of: DNAJA1, HNRNPA2B1 (top left to right), PHIP, SYNE2 (2^{nd} row, left to right), YTHDC1, and ZFC3H1 (bottom left to right). FIG. 4 shows the separation using the classifier.

As can be seen in FIGS. 2A-2C, the following gene products were specifically elevated in fecal samples of patients diagnosed with GI inflammation characterized by MAYO/SESCD score 2 or 3 (herein designated “Group C gene products”):

AC007192.1, ACSL1, ALOX5AP, AQP9, BCL6, CCL4, CD44, CSF3R, CXCL8, DEFA1B, FAM129A, FCGR2A, FCGR3B, FPR1, FYB1, GOS2, GBP1, GMFG, HCAR2, HCAR3, ICAM1, IFI16, IFITM2, IL1B, IL1RN, ITGAX, LCP1, LCP2, LILRB3, LYN, MNDA, NAMPT, NCF2, OSM, PDE4B, PFKFB3, PLEK, PPIF, PROK2, PTGS2, S100A4, S100A9, SAMSN1, SLC2A3, SOCS3, SOD2, SRGN, TNFAIP6, TREM1, and ZNF267.

These genes (as shown in FIGS. 2A-2C) have a median normalized expression in the samples with MAYO/SESCD score 2 or higher class that is higher than 5*10-4, and 5-fold higher than the median normalized expression in the samples with MAYO/SESCD score lower than 2. The summed normalized expression of these markers, denoted “sum23”, was computed by division of the UMI counts of each gene product by the sum of normalized UMI counts of all of these genes. The normalized UMI counts are obtained for each sample and gene by dividing the UMI count of each gene by the sum of UMI counts of all genes in that sample.

In addition, FIGS. 3A-3B shows that the following gene products were specifically elevated in fecal samples of patients diagnosed with GI inflammation characterized by MAYO/SESCD score 0 or 1 (herein designated “Group D gene products”):

### CAPZA1, CASP5, DNAJA1, HNRNPA2B1, ITSN2, KIAA1109, PHIP, SYNE2, TANK, TRA2B, YTHDC1, and ZFC3H1.

These genes (as shown in FIGS. 3A-3B) have a median normalized expression in the samples with MAYO/SESCD score 0/1 class that is higher than 1*10⁻⁴, and 1.2-fold higher than the median normalized expression in the samples with no inflammation. The summed normalized expression of these markers, denoted “sum01”, was similarly computed as specified above.

Next, the variable sum23/(sum23+sum01) was computed, and the ability of this variable to serve as a classifier for analyzing disease severity was evaluated. As can be seen in FIG. 4, the classifier provided remarkable separation of samples by endoscopic severity. In particular, values between 0.4 and 0.75 were determined to indicate low severity (MAYO/SESCD 0 or 1) and values above 0.75 were determined to indicate high severity (MAYO/SESCD 2 or 3). Thus, a classifier providing an unexpectedly improved ability to identify accurately the severity of GI inflammation in patients afflicted with various forms of IBD was identified.

### Example 3. Preparation and Analysis of Stool Samples

Stool collection-Stool samples were collected into 30 ml tubes with built-in spoons, filled with 25 ml RNAlater. Patient were instructed to collect small specimens from the end of the stool that was excreted last from the bowels. Size of specimens was small (equal in size to a pea) with preferred soft consistency. Tubes containing the collected samples were subjected to two storage methods. In one group (herein denoted “new storage”), samples were cryopreserved within one hour of collection, and stored at −20° C. (for short-term use within up to 24 hr of collection) or −80° C. (for long-term storage of more than one day). In another group (denoted “old storage”), samples were stored for up to 24 hr of collection at room temperature, and then at −80° C., as previously described.

RNA isolation-Frozen tubes were thawed lying on ice for approximately 1 hr. Stool samples were taken from the RNAlater and rolled to dry on kim-wipe paper. Stool samples were lyzed in 1 ml RLT buffer containing 50 mM DTT with RNA dissociation beads in a bullet blender (3 min speed 8). After dissociation, tubes were centrifuged at 5000 g for 3 min at 4° C. to eliminate solids. Supernatant was moved to new 1.5 ml Eppendorf tube and RNA was isolated with micro RNeasey kit (Qiagen) according to the manufacturer instruction and eluted in 25 μl RNA pure water.

Library preparation-RNA was depleted from bacterial ribosomal RNA (rRNA) using the NEBNext® rRNA Depletion Kit (Bacteria) according to the manufacturer instruction and eluted in 9 μl RNA pure water. Depleted RNA samples were used for library construction according to a protocol based on RNA barcoding and sequencing, as follows.

Reverse transcription (RT) reaction was performed with a final reaction volume of 20 μl (1× Maxima H Buffer, 1 mM dNTPs, 2 μM TSO* E5V6NEXT (ACACTCTTTCCCTACACGACGCrGrGrG, SEQ ID NO: 1, wherein “rG” represents riboguanidine), 7.5% PEG8000, 40U Maxima H enzyme, 2 μl barcoded RT primer Biotin-ACACTCTTTCCCTACACGACGCTCTTCCGATCT-[BC6][UMI10][T30]VN, Formula I, wherein “BC6” represents 6-mer oligonucleotide barcodes, “UMI10” represents UMIs containing 10-mer random nucleotides, “T30” represents 30-mer oligo dT, and “VN” represents random oligonucleotides (N), and oligonucleotides comprising A, C, or G (V), respectively, SEQ ID NO: 2-33). Barcoded cDNA was cleaned up using SPRI beads at a ratio of 1:1. Purified cDNA was eluted in 17 μl and residual primers digested with Exonuclease I (Thermo Fisher) for 20 min at 37° C. After heat inactivation for 10 min at 80° C., 30 μl PCR master mix consisting of 1.25 U Terra direct polymerase (Clontech) 1.66× Terra direct buffer and 0.33 μM SINGV6 primer (IDT) was added (Biotin-ACACTCTTTCCCTACACGACGC, SEQ ID NO: 34). PCR was cycled as follows: 3 min at 98° C. for initial denaturation followed by 16 cycles of 15 sec at 98° C., 30 sec at 65° C., and 4 min at 68° C. Final elongation was performed for 10 min at 72° C. Following preamplification, all samples were purified using SPRI beads at a ratio of 1:0.8 with a final elution in 10 μl of H₂O (Invitrogen). The cDNA was used to construct Nextera XT libraries from 1 ng of preamplified cDNA. During library PCR, 3′ ends were enriched with a custom unique indexed P5 primer (P5NEXTPT5, IDT AATGATACGGCGACCACCGAGATCTACAC[110]ACACTCTTTCCCTACACGACGCTCT TCCG*A*T*C*T, SEQ ID NO: 35-82, wherein “110” represents unique dual index (UDI) barcodes compatible with Illumina equipment (Illumina UDI), and asterisks represent phosphorothioate bonds). Library final concentration of 1.8 nM was loaded on Novaseq 6000 (Illumina) sequencing machine aiming at 20 M reads per sample with the following setting: Read1-16 bp, Index1-10 bp, Index 2-10 bp, Read2-66 bp.

Gene UMIs were determined as above, and the results are presented in FIGS. 5A-5B. In particular, FIG. 5A illustrates the fraction of samples that passed the detection threshold (minimum UMI count sufficient to enter the analysis). FIG. 5B shows the median UMI count of all samples that entered the analysis.

As can be seen in FIGS. 5A-5B, the modified protocol, including a collection step comprising prompt cryopreservation, followed by removal of rRNA (5S, 16S and 23S) from gram-positive and gram-negative organisms by RNase H-based RNA depletion, generation of an cDNA library using RNA barcoding and sequencing, provided remarkable accuracy in subsequent analyses. In particular, addition of the step of substantially immediate cryopreservation (within less than an hour of sample collection), facilitated substantially improved analyses. In particular, FIG. 5B shows that the modified protocol (new storage) resulted in substantially enhanced UMI counts in the samples. Further, FIG. 5A shows that the enhancement in UMI facilitated the inclusion of a substantially larger portion of samples in subsequent analyses, as more samples were determined to contain sufficiently high UMIs across the detected gene products to meet the detection threshold.

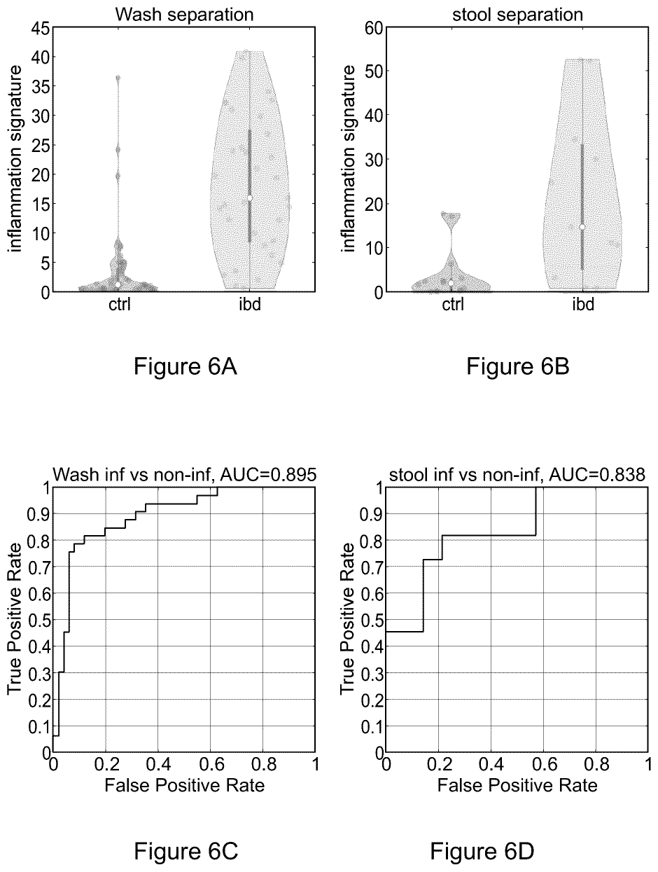

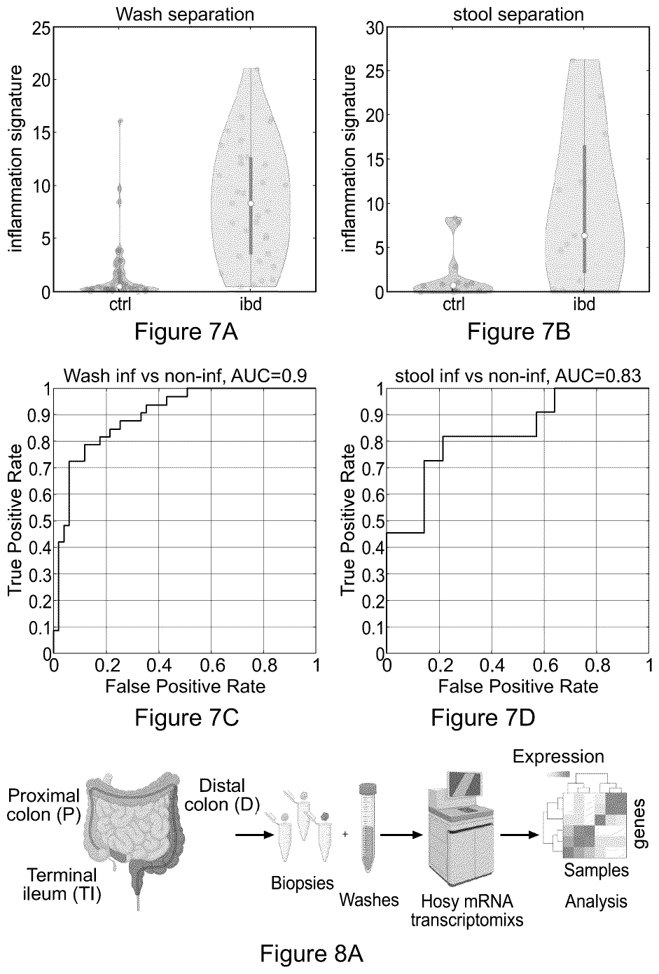

Accordingly, the analyses of stool samples presented in Example 4 below (FIGS. 6A-6D and 7A-7D include analyses generated by the improved protocol specified above.

### Example 4. Improved Classifiers for Active Inflammation

Improved classifiers for identifying active GI inflammation, amenable for use with various types of fecal samples including fecal washes and stool, were further identified by the analyses.

To this end, gene markers that can provide robust classification of inflamed IBD patients were computed based on consensus of differential gene expression (DGE) of distal fecal washes and of stools. Distal fecal wash analysis was based on 51 controls (patients with no inflammation, including both healthy individuals and IBD patients in remission) and 33 IBD patients with active inflammation, and stool DGE analyses were based on 16 stool samples from controls and 9 from IBD patients. Genes considered were those that had UMI-sum normalized expression above 1*10⁻⁴ in both stool and fecal wash datasets (maximal expression across the median of the control and IBD samples). Genes were further considered if their median expression was 1.5-fold higher in IBD compared to control in both stools and distal fecal wash datasets. The minimal fold changes of these genes were next sorted from highest to lowest and the top 75 genes or 35 genes were selected as the gene panel sets.

Next, an inflammation signature for each of these panels was computed as the sum of their maximal and UMI-summed normalized expression as follows. The expression for each gene in each sample was first normalized by dividing by the sum of UMIs of all genes in that sample. The sum-normalized expression was then divided by its maximal expression in all samples, yielding expression between 0 and 1 for each gene. The normalization prevented highly-expressed genes from dominating the signatures. The sum of this max-normalized expression was computed for each sample. The results are presented in FIGS. 6A-6D and 7A-7D.

As can be seen in FIGS. 6A-6D, a classifier incorporating the sum of UMIs of the following 75 gene products provided remarkable diagnostic capacity to identify active inflammation in cohorts including distal colonic washes (84 patients) and stool samples (25 patients):

GBP5, DEFA3, DEFA1B, FCGR3A, CLEC2B, SH3BP5, CD53, CLEC7A, PLAU, IL1RAP, CCR1, FCER1G, CLEC4E, TLR2, LSP1, ADGRG3, CD83, TREM1, CMTM2, RHOH, APBB1IP, LRRK2, BID, KCNJ15, IFIT2, PLEK, PDE4B, HCAR3, ITGAX, SELL, MNDA, HCAR2, ICAM1, FCGR2A, CCLAL2, CCL3L3, GBP1, CSF3R, CD44, PROK2, SOCS3, GMFG, S100A4, TNFAIP6, SNX10, NBN, OSM, SOD2, IFI16, FYB1, AC007192.1, FCGR3B, IL1B, CYTH4, IL1RN, OLR1, VNN2, CCL3, CCL4, IFIT3, CREM, ZEB2, ALOX5AP, CXCL8, LCP2, IGSF6, CXCR2, ZNF267, GBP4, LCP1, PHACTR1, ARRB2, TMEM154, BCL6, and GPR65 (herein designated “Group E gene products”).

As can be seen in FIG. 6C, the classifier provided separation of fecal wash samples form IBD patients with active inflammation from those of healthy controls with an AUC of 0.895. As can be seen in FIG. 6D, the classifier provided separation of stool samples form IBD patients with active inflammation from those of healthy controls with an AUC of 0.838.

A second classifier provided remarkable diagnostic capacity to identify active inflammation using only 35 gene products, in cohorts including both distal colonic washes and stool samples, as can be seen in FIGS. 7A-7D. The following gene product UMIs were included: GBP5, DEFA3, DEFA1B, FCGR3A, CLEC2B, SH3BP5, CD53, CLEC7A, PLAU, IL1RAP, CCR1, FCER1G, CLEC4E, TLR2, LSP1, ADGRG3, CD83, TREM1, CMTM2, RHOH, APBB1IP, LRRK2, BID, KCNJ15, IFIT2, PLEK, PDE4B, HCAR3, ITGAX, SELL, MNDA, HCAR2, ICAM1, FCGR2A, and CCL4L2 (herein designated “Group F gene products”).

As can be seen in FIG. 7C, the classifier provided separation of fecal wash samples form IBD patients with active inflammation from those of healthy controls with an AUC of about 0.9. As can be seen in FIG. 7D, the classifier provided separation of stool samples form IBD patients with active inflammation from those of healthy controls with an AUC of about 0.83.

Accordingly, the improved classifiers identified provided highly accurate means to identify and characterize GI inflammation using minimally-invasive and non-invasive assays.

### Example 5. Distal Fecal Wash Host Transcriptomics Identifies Inflammation Throughout the Colon and Terminal Ileum

Host Transcriptomics of Fecal Washes from Different Intestinal Segments Captures Information that is Distinct from Same-Segment Biopsy Transcriptomics

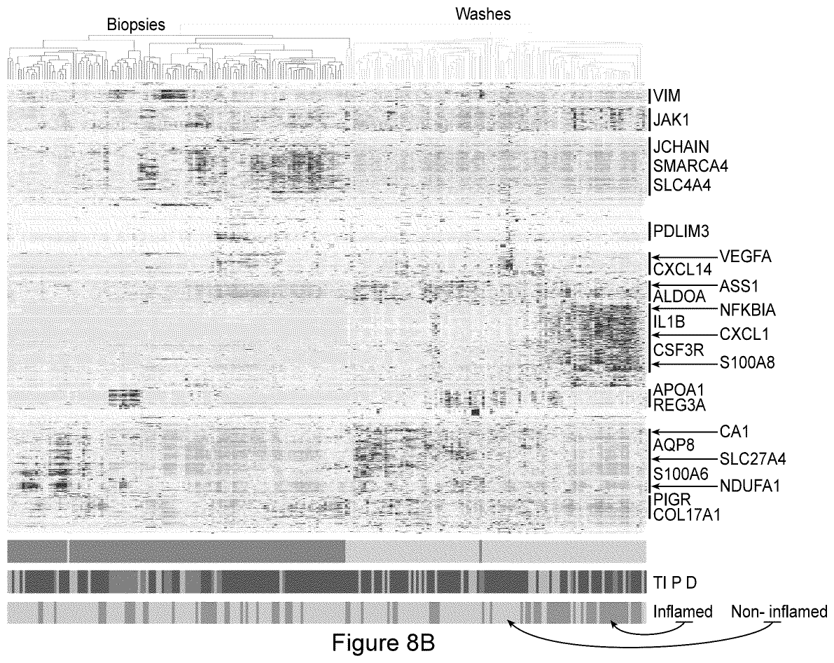

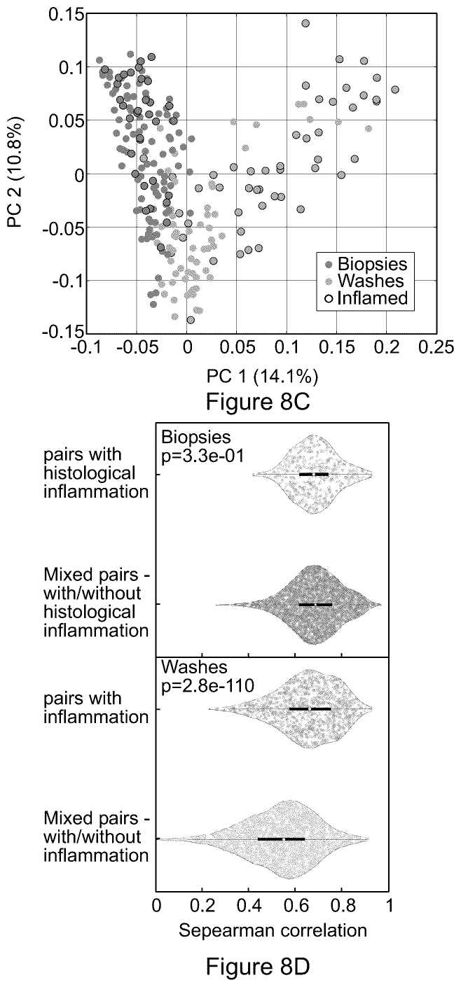

To assess the information captured by fecal wash host transcriptomics in IBD colonoscopies were performed on 59 IBD patients and 50 controls (with no signs of inflammation observed on colonoscopy) (FIG. 8A). Clinical and demographic parameters of the 3 study subgroups (control, CD, and UC) are presented in Table 5. Biopsies and matching fecal washes were sampled from multiple segments: the distal (left sided, sigmoid) colon, proximal (right) colon, and terminal ileum (TI). The cohort included 56 patients and controls from previous study of distal colitis and 53 new patients and controls aimed specifically at enriching for CD pathology without distal involvement (FIG. 9A).

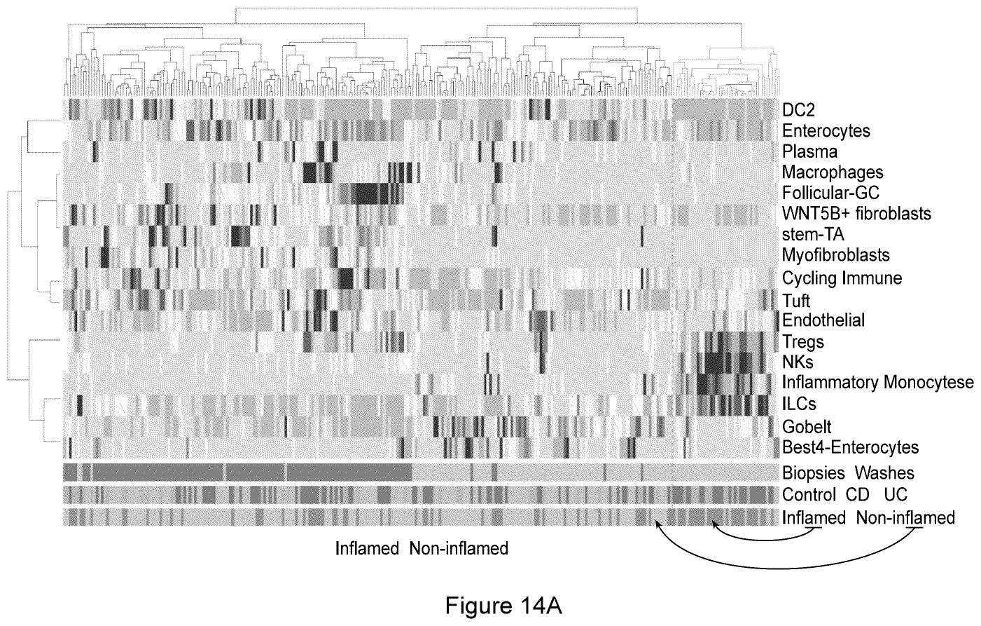

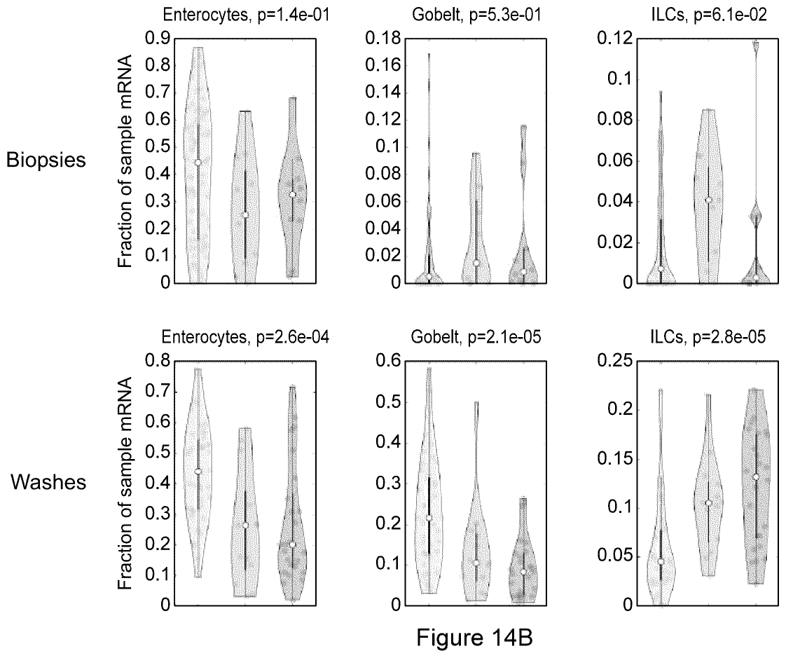

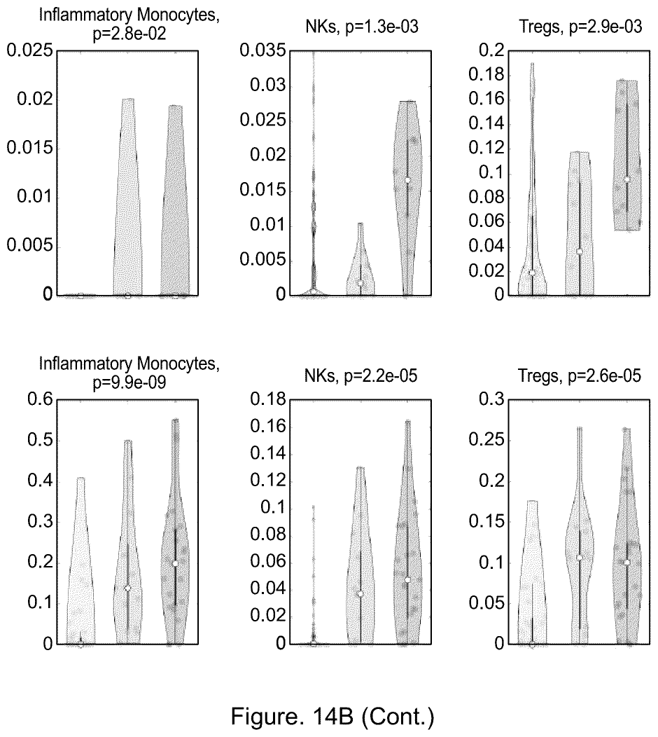

RNA from these samples was sequenced using the mcSCRBseq protocol (see the Methods section), a unique molecular identifier (UMI)-based sensitive protocol ideally suited for low-quality mRNA samples. The reads were mapped to the human genome, resulting in 11,275±2728 genes per biopsy sample and 5973±3134 genes per fecal wash sample. Cluster analysis (FIG. 8B) and principal component analysis (FIG. 8C) of all fecal washes and biopsy specimens showed clear separation between the biopsy and fecal wash samples. Fecal wash samples were enriched in immune-related genes, such as IL1B, CXCL8, and TNFAIP6 (FIG. 13B). Biopsy specimens were enriched in genes associated with stromal components (COL6A3), plasma cells (JCHAIN), and epithelial cells (PIGR). Notably, as previously shown in distal fecal washes of IBD patients with exclusively left-sided (distal) inflammation, pairs of fecal wash samples of patients with histologic inflammation were significantly more correlated compared with mixed pairs, containing 1 sample with and 1 without histologic inflammation (median Spearman correlation, 0.66 vs 0.55, respectively; P=2.8e-110) (FIG. 8D). In contrast, pairs of biopsy specimens with histologic inflammation were as correlated as mixed pairs (median Spearman correlation, 0.68 vs 0.69, respectively; P=3.3e-01) (FIG. 8D). Compared with biopsy specimens, fecal washes of histologically inflamed patients had a significantly higher representation of multiple immune cell populations involved directly in the inflammatory process, including inflammatory monocytes, innate lymphoid cells (ILCs), regulatory T cells (Tregs), and natural killer cells (FIGS. 14A-14B). Fecal wash host transcriptomics therefore contain information that is distinct from biopsies and that correlates with inflammation status.

### Host Transcriptomics of Distal Fecal Washes Captures Active Inflammation in Ileal or Proximal Colonic CD

It was next assessed whether distal fecal washes could identify inflammation in proximal intestinal segments (right colon, TI). When analyzing the host transcriptomics of distal fecal washes of UC and controls, distinct separation between the patients with and without histologic inflammation was observed (FIGS. 9A and 9B). When analyzing CD patients and controls similar separation was observed between patients with and without histologic inflammation (FIGS. 9C and 9D). Notably, patients without distal involvement, including patients with TI inflammation and no colonic inflammation, clustered based on the transcriptomics of their distal fecal washes (FIGS. 9C and 9D). Distal fecal washes of inflamed CD patients showed increased levels of inflammatory genes such as NFKBIA, IL1RN, CCR1, IFIT2, and S100A9 (FIG. 9E). A classifier of histologic inflammation was built based on the summed expression of inflammatory signature genes (see the Methods section) and applied to either UC or CD patients' distal fecal washes or distal biopsy specimens of both. Receiver operating characteristic curve analysis showed significantly higher classification power based on the fecal wash transcriptomics compared with biopsies: the area under the curve (AUC) of 0.94±0.07 for UC distal fecal washes and the AUC of 0.94±0.09 for distal fecal washes of CD patients without distal colonic involvement, compared with the AUC of 0.82±0.20 for all distal biopsies (FIG. 9F). Host transcriptomics of distal fecal washes therefore serve as powerful classifiers not only of distal colonic inflammation in UC patients, but also of CD inflammation, including when the inflammatory segments are ileal and no colonic involvement is observed.

### Transcriptomics of Distal Fecal Washes Contain Information Referring to the Inflamed Intestinal Segment

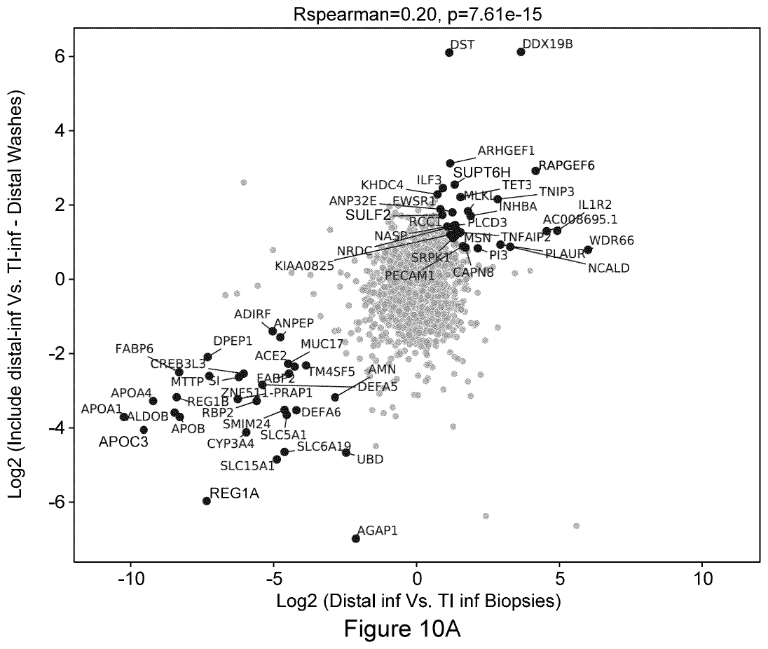

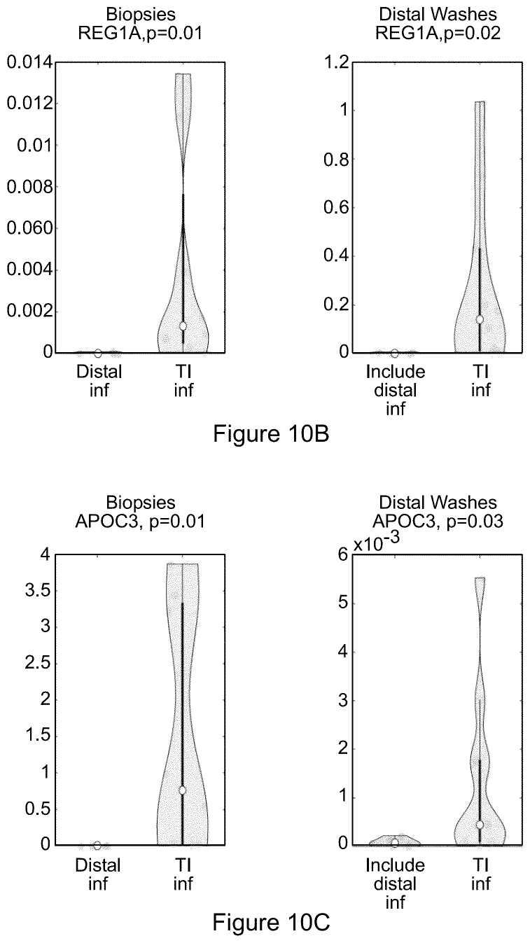

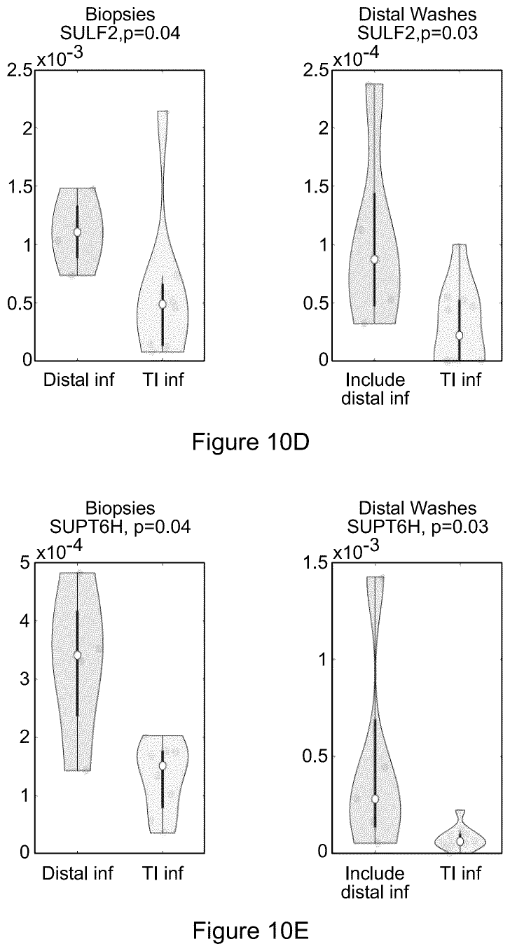

In order to determine whether distal fecal washes contain a signature of the site of inflammation, focusing on patients with active inflammation differential gene expression was performed between distal and ileal inflamed biopsy specimens, as well as between distal fecal washes of patients with TI inflammation only and patients with distal colonic involvement. It was found that both biopsy specimens and distal fecal washes contained differentially expressed genes (Table 4). Notably, the differences in expression were correlated significantly between the distal fecal washes and the biopsy specimens (R=0.20; P=7.61e-15) (FIG. 10A). Genes that showed correlated differences in expression between IBD patients with only TI vs distal colonic inflammation included REG1A (FIG. 10B) and APOC3 (FIG. 10C), which were expressed more highly in patients with TI inflammation, and SULF2 (FIG. 10D) and SUPT6H (FIG. 10E), which were expressed more highly in patients with distal colonic inflammation, both in biopsy specimens and distal fecal washes. Distal colonic fecal washes therefore contain signatures not only of active inflammation but also of the intestinal segment where inflammation occurs.

Distal Fecal Washes Contain Modules of Co-Expressed Immune, Stromal, and Epithelial Genes Associated with Inflammation Severity

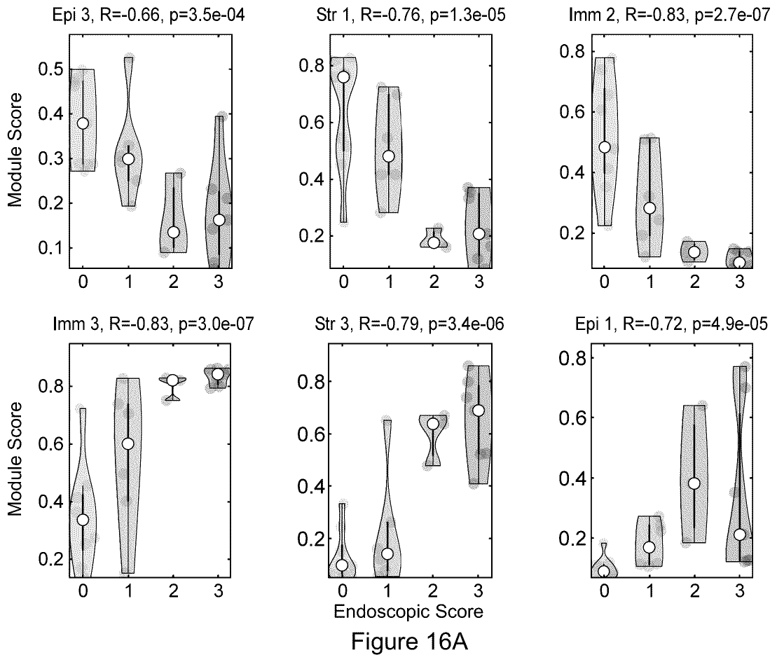

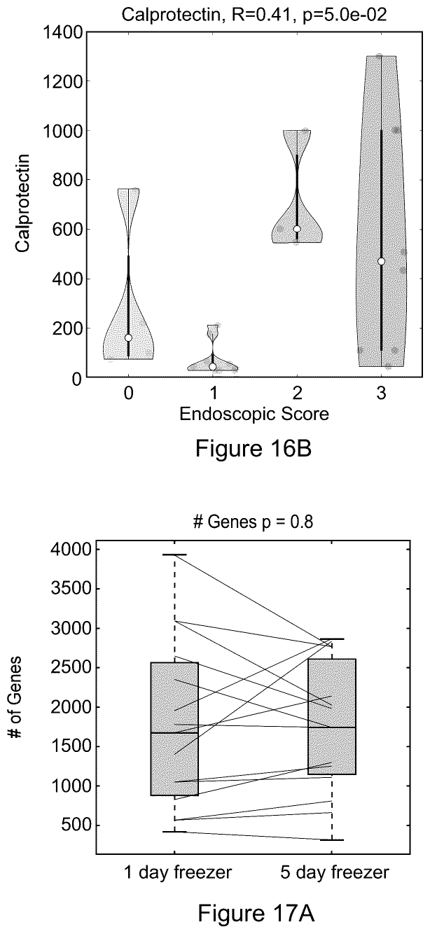

The computational deconvolution showed enriched representation of distinct immune cell types in fecal washes of patients with histologic inflammation. Importantly, other cellular components also were represented in fecal washes, including epithelial and stromal cell types (FIGS. 14A-14B). To explore the transcriptomic landscape of immune, stromal, and epithelial genes in inflamed and noninflamed patients, a single-cell atlas of the human colon was used to extract genes that are expressed solely by these coarse-grained cell types (Methods section). The distal fecal wash samples were clustered based on these distinct gene sets (FIGS. 11A-11E). Unbiased clustering showed several modules of co-expressed genes for each of these cellular subsets. Each sample was assigned a module score based on the summed expression of the respective epithelial (Epi), stromal (Str) and Immune (Imm) module genes. Epithelial genes clustered into 4 modules, including a module enriched in inflamed samples (Epi1, median module score of 0.2 in inflamed fecal washes vs 0.087 in noninflamed fecal washes; P=3.3e⁻⁰³). The Epi1 module was enriched for interleukin (IL) 2/signal transducer and activator of transcription (STAT) 5, transforming growth factor β, and P53 signaling pathways and contained genes previously associated with IBD, including DUOXA2, SAA1, and IL13RA1 (FIG. 11A). Stromal genes clustered into 3 modules, including a module enriched in inflamed samples (Str3, median module score of 0.5 in inflamed fecal washes vs 0.08 in noninflamed fecal washes; P=2.8e-03). Str3 included genes previously shown to be expressed in inflammatory fibroblasts, such as CHI3L1, CCL2, MMP3, and IFITM3 (FIG. 11B). Immune genes clustered into 3 modules, including a module enriched in inflamed samples (Imm3, median module score of 0.8 in inflamed fecal washes vs 0.32 in noninflamed fecal washes; P=1.6e-03), which contained genes previously associated with intestinal inflammation, such as CXCR4, CSF3R, IL1B, and OSM (FIG. 11C). Both Str3 and Imm3 modules were enriched in inflammation-associated pathways such as interferon γ, tumor necrosis factor α (TNF-α), IL6/Janus kinase/STAT3, and IL2/STAT5 signaling. It was found that patients clustered according to their module scores, with increased combined expression of Str3, Imm3, and Epi1 in the distal fecal washes of patients with histologic inflammation (FIG. 11D). Notably, the module scores showed trends that were correlated with the severity of inflammation, as computed by clinical, inflammatory, and endoscopic parameters (Methods section, FIGS. 11D and 15, Table 6). For example, stromal modules Str3 and Str1 increased or decreased, respectively, with inflammation severity in a gradual manner, Epi3 began to decrease in patients with moderate inflammation, and Str2 peaked in patients with mild inflammation (FIGS. 11D and 15). When considering only endoscopic score as a proxy for severity, it was further found that module scores such as Imm3 and Str3 increased monotonically (R=0.83, P=3.0e⁻⁰⁷ and R=0.79, P=3.4e⁻⁰⁶, respectively) (FIG. 16A). In contrast, calprotectin levels changed nonmonotonically and showed substantially decreased correlation with endoscopic severity (R=0.41; P=5.0e⁻⁰²) (FIG. 16B). The analysis therefore highlights modules of epithelial, stromal, and immune genes co-expressed in shed cells that are enriched in patients with histologic inflammation and are highly associated with endoscopic inflammation severity.

Modules of Co-Expressed Genes that Correlate with Response to Biological Therapy Carry Information on Inflammation Severity in Fecal Washes

A recent study of biopsy transcriptomics in IBD patients by Powric and colleagues (Friedrich et al. 2021, ibid) defined gene modules that were shown to correlate with either response or lack of response to biological therapies (anti-TNF and anti-integrin) and to corticosteroids. The inventors sought to explore the expression of these modules in the distal fecal washes (FIG. 12A) and biopsy specimens (FIG. 12B) of their cohort. A module score was generated for each sample based on the sum of the respective module genes and clustered the data. It was found that distal fecal washes of inflamed patients clustered based on this module sum signature (FIGS. 12A and 12C). Patients with histologic inflammation had higher expression of modules M1, M2, M4, and M5 (FIG. 12D), which previously were shown to correlate with IBD severity. Notably, the difference in M1, M4, and M5 module scores was significantly higher in the distal fecal washes compared with the biopsy specimens (FIGS. 12D and E). When clustering only the inflamed distal fecal washes, based on the module scores, two clusters of patients were identified, one of which strongly enriched in modules that corresponded to nonresponse to the aforementioned therapies (M4, M5) (FIG. 12C). Notably, this cluster was enriched in patients with more severe disease status, as determined by clinical, inflammatory, and endoscopic parameters (13 of 15 samples, hypergeometric P value of 4.5e⁻⁰⁵) (Methods section, Table 6). The analysis presented herein indicates that distal fecal washes, including from CD patients with no distal colitis, contain a strong transcriptomic signature of gene modules associated with response to biological therapy that correlate with disease severity.

### Example 6. Extended Storage of Stool Samples in Domestic Freezers

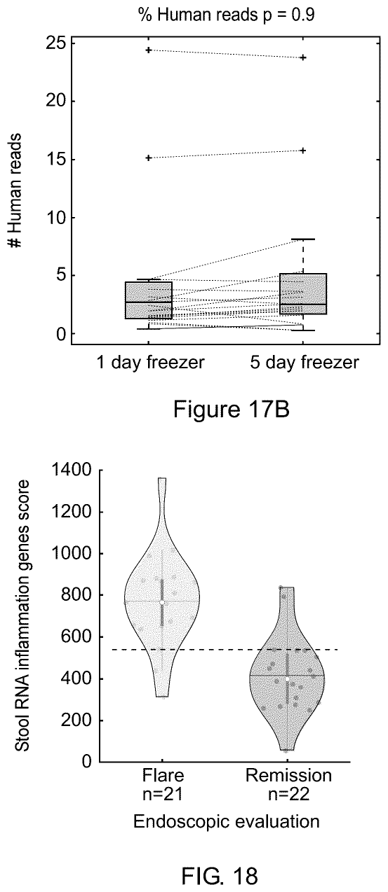

To evaluate the quality of stool samples subjected to immediate cryopreservation over extended storage periods in domestic freezers, stool samples were collected and processed according to the improved protocol described in Example 3, with different storage times at −20° C. In particular, samples were cryopreserved at −20° C. within one hour of collection, and maintained in RNAlater buffer at −20° C. for either 24 hours or five days, before being subjected to RNA recovery (sample thawing and RNA isolation as described above) and subsequent processing using a human fecal transcriptomics protocol as described in Example 3. FIGS. 17A-17B show the results of analyses of the number and proportion of detectable human genes in 15 stool samples of healthy subjects. The boxes represent the interquartile range (IQR), with the horizontal line indicating the median. Whiskers extend to data points within 1.5 times the IQR, and outliers are shown as individual points. Dashed lines connect matched samples between the two groups.

As can be seen in FIG. 17A, extension of the storage time at −20° C. from one day to 5 days did not significantly change the number of human genes detected, wherein values ranged from a median of 1674±1073 for one day storage to a median of 1745±834 for 5 days storage (ranksum p-value=0.8). In addition, as can be seen in FIG. 17B, extension of the storage time at −20° C. from one day to 5 days did not significantly change the percentage of reads mapped to the human genome (2.69% for 1-day freezer storage, 2.51% for 5-day freezer storage; ranksum p-value=0.9).

These results further confirm that a modified protocol including a collection step comprising prompt cryopreservation (within one hour of collection), followed by removal of rRNA from gram-positive and gram-negative organisms by RNase H-based RNA depletion, generation of an cDNA library using RNA barcoding and sequencing, provided remarkable accuracy in subsequent analyses. The results also indicate that prompt cryopreservation enabled enhanced accuracy wherein the samples could conveniently be maintained at a temperature not higher than −20° C. for up to five days or more before RNA recovery.

Accordingly, additional analyses were performed on stool samples obtained from IBD patients and healthy controls, which were stored and processed according to the extended (5-day) storage protocol described above. In particular, stool samples from 21 IBD patients experiencing flare-ups, and 22 patients with no active GI inflammation (including both healthy controls or IBD patients in remission) were obtained and processed according to the extended storage protocol. UMIs were obtained and normalized essentially as described in Example 4, wherein sequencing was performed using NovaseqX and analysis was performed by analyzed by UTAP. FIG. 18 depicts violin plots showing the distribution of RNA scores (multiplied by 106 for display) with individual data points for patients with IBD flare-ups (left) and remission/no inflammation (right).

As can be seen in FIG. 18, a classifier was generated that remarkably differentiated between flare and remission states with 90% sensitivity and specificity. The classifier included the following gene products: RNASEK, RNASEK-C17orf49, MIDN, HLA-A, B2M, HLA-B, HLA-C, CAP1, PFN1, ACTB, MXD1, SAT1, LITAF, NFKBIA, S100A9, S100A8, EIF1, FTL, FTH1 (herein designated “Group G gene products”).

In a further evaluation, data was categorized based on endoscopic remission status (yes/no), with healthy patients included in the remission group, resulting in almost two equal groups (21 remission, 22 non-remission/inflammation). The data was randomly split into a training set including 70% of the samples and a test set including 30% of the samples, evaluated for 10 iterations. In each iteration, a differential gene expression (DGE) analysis was performed using a T-test, and a set of differentially expressed genes was recorded for each group. Genes were included in the list only if they appeared in 90% of the iterations. The resulting gene signatures included those identified as Group H and I gene products (for genes up-regulated in GI inflammation as compared to non-inflamed controls) and Group J gene products (for genes down-regulated in GI inflammation as compared to non-inflamed controls), as detailed in Table 3 above.

In summary, described herein is an improved stool processing protocol that may be conveniently applied for outpatients using domestic freezers, for non-invasive evaluation of GI inflammation. The construction of highly accurate diagnostic classifier that could differentiate active inflammation outbreaks (such as IBD flares) not only from healthy controls but also from IBD patients in remission, is further demonstrated. Without wishing to be bound by a specific theory or mechanism of action, gene products and signatures resulting from the analyses described above may be particularly useful when using stool samples, including those subjected to prolonged storage in domestic freezers.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

What is claimed is:

1. A method of analyzing a fecal RNA sample, comprising:
i. providing a fecal RNA sample, by a method comprising:
a. providing a frozen stool sample that had been stored at a temperature not higher than-20° C. within 1 hour of sample collection,
b. recovering RNA from said sample to provide a fecal RNA sample, and
c. subjecting said fecal RNA sample to selective depletion of microbial rRNA, and
ii. determining the level of at least one human gene product in the resulting fecal RNA sample.

2. The method of claim 1, comprising:
i. providing a fecal RNA sample, by a method comprising:
a) providing a stool sample of a subject afflicted with, or suspected of having, gastrointestinal (GI) inflammation,
b) storing the stool sample at a temperature not higher than −20° C. within 1 hour of sample collection,
c) recovering RNA from said sample to provide a fecal RNA sample, by thawing the frozen sample and isolating RNA from the thawed sample,
d) subjecting said RNA sample to RNase H-based selective depletion of 5S, 16S and 23S rRNA of gram-positive and gram-negative bacteria,
e) performing reverse transcription of the resulting depleted RNA, generating a library of gene products using RNA barcoding, and sequencing, and
ii. determining the level of at least one human gene product in the fecal RNA sample.

3. The method of claim 2, wherein the subject is diagnosed with, or suspected of having, an inflammatory bowel disease (IBD).

4. The method of claim 3, wherein step (ii) comprises determining the levels in the sample of a plurality of the human gene products, thereby obtaining the transcriptomic signature of the sample with respect to the plurality of gene products.

5. The method of claim 4, further comprising comparing the transcriptomic signature of said sample to a control transcriptomic signature by a supervised classification algorithm.

6. The method of claim 4, wherein the plurality of human gene products is selected from the group consisting of:
AC007192.1, ACSL1, ADGRG3, ALDOB, ALOX5AP, ALPL, AMN, ANP32E, AOC1, APBB1IP, APOA1, APOA4, APOB, APOC3, AQP9, ARHGAP26, ARHGAP30, ARRB2, BCL6, BID, CAPZA1, CASP5, CCL3, CCL3L3, CCL4, CCL4L2, CCR1, CD44, CD53, CD83, CDHR2, CDHR5, CLEC2B, CLEC4E, CLEC7A, CMTM2, CREB3L3, CREM, CSF3R, CXCL8, CXCR2, CYP3A4, CYTH1, CYTH4, DDX21, DEFA1B, DEFA3, DEFA5, DEFA6, DNAJA1, DPEP1, EIF3J, EPS8L1, ERICH1, EWSR1, FABP2, FABP6, FAM129A, FCER1G, FCGR2A, FCGR3A, FCGR3B, FNBP1, FPR1, FYB1, GOS2, GBP1, GBP4, GBP5, GDF5OS, GMFG, GNA11, GPR65; GUCA2A, GUCA2B, HCAR2, HCAR3, HNRNPA2B1, ICAM1, IFI16, IFIT2, IFIT3, IFITM2, IGSF6, IL1B, IL1R2, IL1RAP, IL1RN, ILF3, ITGAX, ITSN2, KCNJ15, KCNK6, KIAA0825, KIAA1109, LCP1, LCP2, LILRB3, LRRK2, LSP1, LYN, MAVS, MEP1A, METAP2, MNDA, MTRNR2L1, MTTP, NAMPT, NBN, NCF2, NCL, OLR1, OSM, PDE4B, PFKFB3, PHACTR1, PHIP, PLAU, PLCB2, PLCD3, PLEK, PPIF, PROK2, PTGS2, RAPGEF6, RCC1, REG1A, REG1B, RHOH, S100A4, S100A9, SAMSN1, SELENOP, SELL, SH3BP5, SI, SIPA1L2, SLC15A1, SLC2A3, SLC5A1, SMIM24, SNX10, SOCS3, SOD2, SP140, SRGN, SUPT6H, SYNE2, TANK, TET3, TLR2, TM4SF5, TMEM154, TNFAIP6, TRA2B, TREM1, UTRN, VNN2, WDR66, YTHDC1, ZEB2, ZFC3H1, ZNF267, and ZNF511-PRAP1.

7. The method of claim 4, wherein said plurality of human gene products is selected from the group consisting of:
a. TET3, RAPGEF6, RCC1, IL1R2, WDR66, ANP32E, EPS8L1, ALPL, ILF3, ERICH1, PLCD3, MAVS, SIPA1L2, ARHGAP30, FNBP1, NCL, EWSR1, SP140, DDX21, KCNK6, KIAA0825, SUPT6H, CYTH1, EIF3J, ARHGAP26, MTRNR2L1, PLCB2, UTRN, METAP2, and GDF5OS (Group A gene products);
b. REG1A, FABP6, REG1B, APOB, ALDOB, ZNF511-PRAP1, SI, CYP3A4, APOA1, DEFA6, APOA4, DPEP1, DEFA5, APOC3, CREB3L3, AMN, SLC15A1, GUCA2A, SMIM24, MTTP, SLC5A1, FABP2, MEP1A, TM4SF5, GUCA2B, AOC1, CDHR5, CDHR2, SELENOP, and GNA11 (Group B gene products);
c. AC007192.1, ACSL1, ALOX5AP, AQP9, BCL6, CCL4, CD44, CSF3R, CXCL8, DEFA1B, FAM129A, FCGR2A, FCGR3B, FPR1, FYB1, GOS2, GBP1, GMFG, HCAR2, HCAR3, ICAM1, IFI16, IFITM2, IL1B, IL1RN, ITGAX, LCP1, LCP2, LILRB3, LYN, MNDA, NAMPT, NCF2, OSM, PDE4B, PFKFB3, PLEK, PPIF, PROK2, PTGS2, S100A4, S100A9, SAMSN1, SLC2A3, SOCS3, SOD2, SRGN, TNFAIP6, TREM1, and ZNF267 (Group C gene products);
d. CAPZA1, CASP5, DNAJA1, HNRNPA2B1, ITSN2, KIAA1109, PHIP, SYNE2, TANK, TRA2B, YTHDC1, and ZFC3H1 (Group D gene products);
e. GBP5, DEFA3, DEFA1B, FCGR3A, CLEC2B, SH3BP5, CD53, CLEC7A, PLAU, IL1RAP, CCR1, FCER1G, CLEC4E, TLR2, LSP1, ADGRG3, CD83, TREM1, CMTM2, RHOH, APBB1IP, LRRK2, BID, KCNJ15, IFIT2, PLEK, PDE4B, HCAR3, ITGAX, SELL, MNDA, HCAR2, ICAM1, FCGR2A, CCLAL2, CCL3L3, GBP1, CSF3R, CD44, PROK2, SOCS3, GMFG, S100A4, TNFAIP6, SNX10, NBN, OSM, SOD2, IFI16, FYB1, AC007192.1, FCGR3B, IL1B, CYTH4, IL1RN, OLR1, VNN2, CCL3, CCL4, IFIT3, CREM, ZEB2, ALOX5AP, CXCL8, LCP2, IGSF6, CXCR2, ZNF267, GBP4, LCP1, PHACTR1, ARRB2, TMEM154, BCL6, and GPR65 (Group E gene products); and/or
f. GBP5, DEFA3, DEFA1B, FCGR3A, CLEC2B, SH3BP5, CD53, CLEC7A, PLAU, IL1RAP, CCR1, FCER1G, CLEC4E, TLR2, LSP1, ADGRG3, CD83, TREM1, CMTM2, RHOH, APBB1IP, LRRK2, BID, KCNJ15, IFIT2, PLEK, PDE4B, HCAR3, ITGAX, SELL, MNDA, HCAR2, ICAM1, FCGR2A, and CCLAL2 (Group F gene products).

8. The method of claim 4, wherein said plurality of human gene products is selected from the group consisting of:
a. RNASEK, RNASEK-C17orf49, MIDN, HLA-A, B2M, HLA-B, HLA-C, CAP1, PFN1, ACTB, MXD1, SAT1, LITAF, NFKBIA, S100A9, S100A8, EIF1, FTL, and FTH1 (Group G gene products),
b. AC138811.2, AQP9, ARPC2, ARPC5, BASP1, BCL2A1, BRI3, BTG2, CALM2, CCL4, CDKN1A, CEACAM1, CEBPB, CXCL8, EGR1, ETS2, FOS, FPR1, FTL, GOS2, GABARAP, GLUL, GNB2, HCAR3, HLA-C, HLA-E, ICAM1, IFITM1, IFITM2, IL1B, IL1RN, IRF1, ISG20, ITM2B, IVNS1ABP, KDM6B, KLF6, LITAF, MARCKS, MCL1, MXD1, NAMPT, NFKBIA, OSM, PFN1, PLAUR, PLEK, PNRC1, PPIF, PROK2, PTP4A1, S100A11, S100A8, S100A9, SAT1, SDCBP, SLC25A37, SOCS3, SOD2, SRGN, TAF10, TNFAIP3, TPM4, TXNIP, TYMP, UBE2B, VASP, WDR83OS, ZFP36, and ZFP36L1 (Group H gene products),
c. CCL4, CXCL8, HCAR3, ICAM1, IL1B, IL1RN, OSM, PLEK, PROK2, SOCS3, SOD2 (Group I gene products), and/or
d. ABHD17C, AC005943.1, ACTN4, ALDOA, AP000350.4, AP000721.1, AP003419.1, C17orf49, CA4, CDHR5, CFL1, CKB, COX7A2, COX8A, CRIP1, CST3, CTNND1, DBNDD2, DYNLRB1, EGLN1, EIF4G2, EPCAM, FABP1, FCGBP, FXYD3, GUCA2A, GUCA2B, IFI27, KRT8, LGALS4, LYPD8, MAL2, MGLL, MIF, MISP, MUC12, MUC2, MYL12B, OST4, P2RX5-TAX1BP3, PDLIM1, PHGR1, PIGR, PLAC8, POLD4, PPDPF, RHOC, S100A16, SDCBP2, SERINC2, SFN, SH3BGRL3, SMIM22, SPINT2, SRI, STK24, SYS1-DBNDD2, TAX1BP3, TFF1, TFF3, TMEM54, TMPRSS2, TMSB10, TRIM31, UBA52, UBB, UQCR11, ZG16 (Group J gene products).

9. The method of claim 5, wherein the outcome of the comparison is indicative of the location of GI inflammation in said subject, and said plurality of human gene products is as set forth in Group A and/or B.

10. The method of claim 5, wherein the outcome of the comparison is indicative of the severity of GI inflammation in said subject, and said plurality of human gene products is as set forth in Group C and/or D.

11. The method of claim 5, wherein the outcome of the comparison is indicative of the presence or absence of GI inflammation in said subject, and said plurality of human gene products is as set forth in Group E, F, G, H, I and/or J.

12. The method of claim 5, wherein the plurality of gene products further comprises at least one additional gene product selected from Table 4.

13. The method of claim 3, wherein the subject is afflicted with, or suspected of having, ulcerative colitis (UC), or wherein the subject is afflicted with, or suspected of having, Crohn's disease (CD).

14. The method of claim 1, wherein determining the levels of each gene product in the sample comprises determining the Unique Molecular Identifier (UMI) counts for each gene product.

15. The method of claim 14, wherein determining the levels of each gene product in the sample further comprises normalizing the level of each UMI in said sample to the levels of other UMIs in said sample.

16. The method of claim 1, wherein step (ii) comprises determining the levels in the sample of a plurality of the human gene products, thereby obtaining the transcriptomic signature of the sample with respect to the plurality of gene products wherein said plurality of gene products is selected from Table 1 or from Table 4.

17. The method of claim 1, wherein the stool sample is stored at a temperature not higher than-20° C. within 1 hour of sample collection in the presence of an RNA preservation reagent comprising ammonium sulfate and ethylenediaminetetraacetic acid (EDTA) and in the absence of a cryoprotectant.

18. A method of analyzing a fecal RNA sample, comprising:
i. providing a fecal RNA sample of a subject afflicted with, or suspected of having, GI inflammation,
ii. determining the levels in the sample of a plurality of human gene products, thereby obtaining the transcriptomic signature of the sample with respect to the plurality of gene products, and
iii. comparing the transcriptomic signature of said sample to a control transcriptomic signature by a supervised classification algorithm,
wherein said plurality of gene products is selected from the group consisting of:
a. TET3, RAPGEF6, RCC1, IL1R2, WDR66, ANP32E, EPS8L1, ALPL, ILF3, ERICH1, PLCD3, MAVS, SIPA1L2, ARHGAP30, FNBP1, NCL, EWSR1, SP140, DDX21, KCNK6, KIAA0825, SUPT6H, CYTH1, EIF3J, ARHGAP26, MTRNR2L1, PLCB2, UTRN, METAP2, and GDF5OS (Group A gene products);
b. REG1A, FABP6, REG1B, APOB, ALDOB, ZNF511-PRAP1, SI, CYP3A4, APOA1, DEFA6, APOA4, DPEP1, DEFA5, APOC3, CREB3L3, AMN, SLC15A1, GUCA2A, SMIM24, MTTP, SLC5A1, FABP2, MEP1A, TM4SF5, GUCA2B, AOC1, CDHR5, CDHR2, SELENOP, and GNA11 (Group B gene products);
c. AC007192.1, ACSL1, ALOX5AP, AQP9, BCL6, CCL4, CD44, CSF3R, CXCL8, DEFA1B, FAM129A, FCGR2A, FCGR3B, FPR1, FYB1, GOS2, GBP1, GMFG, HCAR2, HCAR3, ICAM1, IFI16, IFITM2, IL1B, IL1RN, ITGAX, LCP1, LCP2, LILRB3, LYN, MNDA, NAMPT, NCF2, OSM, PDE4B, PFKFB3, PLEK, PPIF, PROK2, PTGS2, S100A4, S100A9, SAMSN1, SLC2A3, SOCS3, SOD2, SRGN, TNFAIP6, TREM1, and ZNF267 (Group C gene products);
d. CAPZA1, CASP5, DNAJA1, HNRNPA2B1, ITSN2, KIAA1109, PHIP, SYNE2, TANK, TRA2B, YTHDC1, and ZFC3H1 (Group D gene products);
e. GBP5, DEFA3, DEFA1B, FCGR3A, CLEC2B, SH3BP5, CD53, CLEC7A, PLAU, IL1RAP, CCR1, FCER1G, CLEC4E, TLR2, LSP1, ADGRG3, CD83, TREM1, CMTM2, RHOH, APBB1IP, LRRK2, BID, KCNJ15, IFIT2, PLEK, PDE4B, HCAR3, ITGAX, SELL, MNDA, HCAR2, ICAM1, FCGR2A, CCL4L2, CCL3L3, GBP1, CSF3R, CD44, PROK2, SOCS3, GMFG, S100A4, TNFAIP6, SNX10, NBN, OSM, SOD2, IFI16, FYB1, AC007192.1, FCGR3B, IL1B, CYTH4, IL1RN, OLR1, VNN2, CCL3, CCL4, IFIT3, CREM, ZEB2, ALOX5AP, CXCL8, LCP2, IGSF6, CXCR2, ZNF267, GBP4, LCP1, PHACTR1, ARRB2, TMEM154, BCL6, and GPR65 (Group E gene products);
f. GBP5, DEFA3, DEFA1B, FCGR3A, CLEC2B, SH3BP5, CD53, CLEC7A, PLAU, IL1RAP, CCR1, FCER1G, CLEC4E, TLR2, LSP1, ADGRG3, CD83, TREM1, CMTM2, RHOH, APBB1IP, LRRK2, BID, KCNJ15, IFIT2, PLEK, PDE4B, HCAR3, ITGAX, SELL, MNDA, HCAR2, ICAM1, FCGR2A, and CCL4L2 (Group F gene products);
g. RNASEK, RNASEK-C17orf49, MIDN, HLA-A, B2M, HLA-B, HLA-C, CAP1, PFN1, ACTB, MXD1, SAT1, LITAF, NFKBIA, S100A9, S100A8, EIF1, FTL, and FTH1 (Group G gene products),
h. AC138811.2, AQP9, ARPC2, ARPC5, BASP1, BCL2A1, BRI3, BTG2, CALM2, CCL4, CDKN1A, CEACAM1, CEBPB, CXCL8, EGR1, ETS2, FOS, FPR1, FTL, GOS2, GABARAP, GLUL, GNB2, HCAR3, HLA-C, HLA-E, ICAM1, IFITM1, IFITM2, IL1B, IL1RN, IRF1, ISG20, ITM2B, IVNS1ABP, KDM6B, KLF6, LITAF, MARCKS, MCL1, MXD1, NAMPT, NFKBIA, OSM, PFN1, PLAUR, PLEK, PNRC1, PPIF, PROK2, PTP4A1, S100A11, S100A8, S100A9, SAT1, SDCBP, SLC25A37, SOCS3, SOD2, SRGN, TAF10, TNFAIP3, TPM4, TXNIP, TYMP, UBE2B, VASP, WDR83OS, ZFP36, and ZFP36L1 (Group H gene products)
i. CCL4, CXCL8, HCAR3, ICAM1, IL1B, IL1RN, OSM, PLEK, PROK2, SOCS3, SOD2 (Group I gene products); and/or
j. ABHD17C, AC005943.1, ACTN4, ALDOA, AP000350.4, AP000721.1, AP003419.1, C17orf49, CA4, CDHR5, CFL1, CKB, COX7A2, COX8A, CRIP1, CST3, CTNND1, DBNDD2, DYNLRB1, EGLN1, EIF4G2, EPCAM, FABP1, FCGBP, FXYD3, GUCA2A, GUCA2B, IFI27, KRT8, LGALS4, LYPD8, MAL2, MGLL, MIF, MISP, MUC12, MUC2, MYL12B, OST4, P2RX5-TAX1BP3, PDLIM1, PHGR1, PIGR, PLAC8, POLD4, PPDPF, RHOC, S100A16, SDCBP2, SERINC2, SFN, SH3BGRL3, SMIM22, SPINT2, SRI, STK24, SYS1-DBNDD2, TAX1BP3, TFF1, TFF3, TMEM54, TMPRSS2, TMSB10, TRIM31, UBA52, UBB, UQCR11, ZG16 (Group J gene products).

19. The method of claim 18, wherein providing said fecal RNA sample comprises:
a) providing a frozen stool sample that had been stored at a temperature not higher than −20° C. within 1 hour of sample collection,
b) recovering RNA from said sample to provide a fecal RNA sample, and
c) subjecting said fecal RNA sample to selective depletion of microbial rRNA.

20. The method of claim 19, wherein providing said fecal RNA sample comprises:
a) providing a stool sample of a subject afflicted with, or suspected of having, GI inflammation,
b) storing the stool sample at a temperature not higher than −20° C. within 1 hour of sample collection,
c) recovering RNA from said sample to provide a fecal RNA sample, by thawing the frozen sample and isolating RNA from the thawed sample,
d) subjecting said RNA sample to RNase H-based selective depletion of 5S, 16S and 23S rRNA of gram-positive and gram-negative bacteria,
e) performing reverse transcription of the resulting depleted RNA, and
f) generating a library of gene products using RNA barcoding and sequencing the generated library.

21. The method of claim 18, wherein the stool sample had been obtained from a subject afflicted with, or suspected of having, an inflammatory bowel disease (IBD).

22. A method of determining one or more human gene products from a stool sample, comprising:
a. providing a frozen stool sample that has been stored at a temperature not higher than −20° C. within 1 hour of sample collection,
b. recovering RNA from said sample to provide a fecal RNA sample,
c. selectively depleting microbial rRNA from the fecal RNA sample, and
d. analyzing the depleted fecal RNA sample of step c) to determine one or more human gene products therein,
wherein step (d) comprises determining the levels in the sample of a plurality of the human gene products selected from Table 1 or from Table 4, thereby obtaining the transcriptomic signature of the sample with respect to the plurality of gene products.

23. The method of claim 22, wherein the stool sample had been obtained from a subject afflicted with, or suspected of having, gastrointestinal (GI) inflammation.

24. A diagnostic kit, comprising means for specifically determining and quantifying the levels of a plurality of human gene products in a fecal RNA sample, wherein the plurality of human gene products comprises or consists of:
a. TET3, RAPGEF6, RCC1, IL1R2, WDR66, ANP32E, EPS8L1, ALPL, ILF3, ERICH1, PLCD3, MAVS, SIPA1L2, ARHGAP30, FNBP1, NCL, EWSR1, SP140, DDX21, KCNK6, KIAA0825, SUPT6H, CYTH1, EIF3J, ARHGAP26, MTRNR2L1, PLCB2, UTRN, METAP2, and GDF5OS (Group A gene products);
b. REG1A, FABP6, REG1B, APOB, ALDOB, ZNF511-PRAP1, SI, CYP3A4, APOA1, DEFA6, APOA4, DPEP1, DEFA5, APOC3, CREB3L3, AMN, SLC15A1, GUCA2A, SMIM24, MTTP, SLC5A1, FABP2, MEP1A, TM4SF5, GUCA2B, AOC1, CDHR5, CDHR2, SELENOP, and GNA11 (Group B gene products);
c. AC007192.1, ACSL1, ALOX5AP, AQP9, BCL6, CCL4, CD44, CSF3R, CXCL8, DEFA1B, FAM129A, FCGR2A, FCGR3B, FPR1, FYB1, GOS2, GBP1, GMFG, HCAR2, HCAR3, ICAM1, IFI16, IFITM2, IL1B, IL1RN, ITGAX, LCP1, LCP2, LILRB3, LYN, MNDA, NAMPT, NCF2, OSM, PDE4B, PFKFB3, PLEK, PPIF, PROK2, PTGS2, S100A4, S100A9, SAMSN1, SLC2A3, SOCS3, SOD2, SRGN, TNFAIP6, TREM1, and ZNF267 (Group C gene products);
d. CAPZA1, CASP5, DNAJA1, HNRNPA2B1, ITSN2, KIAA1109, PHIP, SYNE2, TANK, TRA2B, YTHDC1, and ZFC3H1 (Group D gene products);
e. GBP5, DEFA3, DEFA1B, FCGR3A, CLEC2B, SH3BP5, CD53, CLEC7A, PLAU, IL1RAP, CCR1, FCER1G, CLEC4E, TLR2, LSP1, ADGRG3, CD83, TREM1, CMTM2, RHOH, APBB1IP, LRRK2, BID, KCNJ15, IFIT2, PLEK, PDE4B, HCAR3, ITGAX, SELL, MNDA, HCAR2, ICAM1, FCGR2A, CCL4L2, CCL3L3, GBP1, CSF3R, CD44, PROK2, SOCS3, GMFG, S100A4, TNFAIP6, SNX10, NBN, OSM, SOD2, IFI16, FYB1, AC007192.1, FCGR3B, IL1B, CYTH4, IL1RN, OLR1, VNN2, CCL3, CCL4, IFIT3, CREM, ZEB2, ALOX5AP, CXCL8, LCP2, IGSF6, CXCR2, ZNF267, GBP4, LCP1, PHACTR1, ARRB2, TMEM154, BCL6, and GPR65 (Group E gene products);
f. GBP5, DEFA3, DEFA1B, FCGR3A, CLEC2B, SH3BP5, CD53, CLEC7A, PLAU, IL1RAP, CCR1, FCER1G, CLEC4E, TLR2, LSP1, ADGRG3, CD83, TREM1, CMTM2, RHOH, APBB1IP, LRRK2, BID, KCNJ15, IFIT2, PLEK, PDE4B, HCAR3, ITGAX, SELL, MNDA, HCAR2, ICAM1, FCGR2A, and CCL4L2 (Group F gene products);
g. RNASEK, RNASEK-C17orf49, MIDN, HLA-A, B2M, HLA-B, HLA-C, CAP1, PFN1, ACTB, MXD1, SAT1, LITAF, NFKBIA, S100A9, S100A8, EIF1, FTL, and FTH1 (Group G gene products),
h. AC138811.2, AQP9, ARPC2, ARPC5, BASP1, BCL2A1, BRI3, BTG2, CALM2, CCL4, CDKN1A, CEACAM1, CEBPB, CXCL8, EGR1, ETS2, FOS, FPR1, FTL, GOS2, GABARAP, GLUL, GNB2, HCAR3, HLA-C, HLA-E, ICAM1, IFITM1, IFITM2, IL1B, IL1RN, IRF1, ISG20, ITM2B, IVNS1ABP, KDM6B, KLF6, LITAF, MARCKS, MCL1, MXD1, NAMPT, NFKBIA, OSM, PFN1, PLAUR, PLEK, PNRC1, PPIF, PROK2, PTP4A1, S100A11, S100A8, S100A9, SAT1, SDCBP, SLC25A37, SOCS3, SOD2, SRGN, TAF10, TNFAIP3, TPM4, TXNIP, TYMP, UBE2B, VASP, WDR83OS, ZFP36, and ZFP36L1 (Group H gene products)
i. CCL4, CXCL8, HCAR3, ICAM1, IL1B, IL1RN, OSM, PLEK, PROK2, SOCS3, SOD2 (Group I gene products); and/or
j. ABHD17C, AC005943.1, ACTN4, ALDOA, AP000350.4, AP000721.1, AP003419.1, C17orf49, CA4, CDHR5, CFL1, CKB, COX7A2, COX8A, CRIP1, CST3, CTNND1, DBNDD2, DYNLRB1, EGLN1, EIF4G2, EPCAM, FABP1, FCGBP, FXYD3, GUCA2A, GUCA2B, IFI27, KRT8, LGALS4, LYPD8, MAL2, MGLL, MIF, MISP, MUC12, MUC2, MYL12B, OST4, P2RX5-TAX1BP3, PDLIM1, PHGR1, PIGR, PLAC8, POLD4, PPDPF, RHOC, S100A16, SDCBP2, SERINC2, SFN, SH3BGRL3, SMIM22, SPINT2, SRI, STK24, SYS1-DBNDD2, TAX1BP3, TFF1, TFF3, TMEM54, TMPRSS2, TMSB10, TRIM31, UBA52, UBB, UQCR11, ZG16 (Group J gene products).

25. The diagnostic kit of claim 24, wherein said plurality of gene products comprises or consists of Group A, B, C, D, E and/or F gene products.

26. The diagnostic kit of claim 24, wherein the means comprise quantitative polymerase chain reaction (qPCR) primers directed to said plurality of human gene products, and optionally the kit optionally further comprises means for providing a fecal RNA sample and/or means for comparing the levels of said plurality of human gene products in the sample to their levels in a control fecal RNA sample.
